SEQUENCE LISTING (157 sequences)

SEQ ID NO: 1 - protein, 7 aa, synthetic construct
GEDSTAP

SEQ ID NO: 2 - protein, 12 aa, synthetic construct
GSAGSAAGSGEF

SEQ ID NO: 3 - protein, 9 aa, synthetic construct
GGSGGGSGG

SEQ ID NO: 4 - protein, 15 aa, synthetic construct
GGGGSGGGGSGGGGS

SEQ ID NO: 5 - protein, 4 aa, synthetic construct
  VARIANT >4: note = The entire sequence of amino acids 1-4 can be repeated one or more times.
GGGG

SEQ ID NO: 6 - protein, 4 aa, synthetic construct
GQAP

SEQ ID NO: 7 - protein, 4 aa, synthetic construct
SOBI

SEQ ID NO: 8 - protein, 15 aa, synthetic construct
EAAAKEAAAKEAAAK

SEQ ID NO: 9 - protein, 8 aa, synthetic construct
  REGION >8: note = The entire sequence of amino acids 1-8 can be repeated one or more times.
  VARIANT 1: note = X can be any amino acid, preferably Ala, Lys, or Glu
  VARIANT 3: note = X can be any amino acid, preferably Ala, Lys, or Glu
  VARIANT 5: note = X can be any amino acid, preferably Ala, Lys, or Glu
  VARIANT 7: note = X can be any amino acid, preferably Ala, Lys, or Glu
XPXPXPXP

SEQ ID NO: 10 - DNA, 25 bp, Scytonema hofmannii
  source 1..25: mol_type = other DNA
ttagacatctccacaaaaggcgtag

SEQ ID NO: 11 - DNA, 25 bp, Anabaena cylindrica
  source 1..25: mol_type = other DNA
cgagtctcctattctccattatata

SEQ ID NO: 12 - DNA, 25 bp, Scytonema hofmannii
  source 1..25: mol_type = other DNA
actacctacttaaatgaaccgcaaa

SEQ ID NO: 13 - DNA, 25 bp, Scytonema hofmannii
  source 1..25: mol_type = other DNA
cgtagagacgtagcaatgctacctc

SEQ ID NO: 14 - DNA, 25 bp, Anabaena cylindrica
  source 1..25: mol_type = other DNA
atagcctttctcactctagttagat

SEQ ID NO: 15 - DNA, 25 bp, Scytonema hofmannii
  source 1..25: mol_type = other DNA
ccaaccccaagcattggtaccgagc

SEQ ID NO: 16 - RNA, 23 nt, synthetic construct
  source 1..23: mol_type = other RNA
gagaagtcatttaataaggccac

SEQ ID NO: 17 - RNA, 23 nt, synthetic construct
  source 1..23: mol_type = other RNA
atagcgatcccttgctgaaaata

SEQ ID NO: 18 - RNA, 23 nt, synthetic construct
  source 1..23: mol_type = other RNA
atagtgaatccgcttattctcag

SEQ ID NO: 19 - RNA, 23 nt, synthetic construct
  source 1..23: mol_type = other RNA
actgcccgtttcgagagtttctc

SEQ ID NO: 20 - RNA, 23 nt, synthetic construct
  source 1..23: mol_type = other RNA
accacctcaagctatgccgccag

SEQ ID NO: 21 - RNA, 23 nt, synthetic construct
  source 1..23: mol_type = other RNA
actatagactatccgggcaatgt

SEQ ID NO: 22 - RNA, 23 nt, synthetic construct
  source 1..23: mol_type = other RNA
accctcttaaactatcccactaa

SEQ ID NO: 23 - RNA, 20 nt, synthetic construct
  source 1..20: mol_type = other RNA
tagtataaacgaacaggatc

SEQ ID NO: 24 - RNA, 20 nt, synthetic construct
  source 1..20: mol_type = other RNA
gaatatcaaacagtttatgc

SEQ ID NO: 25 - RNA, 20 nt, synthetic construct
  source 1..20: mol_type = other RNA
tgctcaccaataccaatacc

SEQ ID NO: 26 - RNA, 20 nt, synthetic construct
  source 1..20: mol_type = other RNA
ttcactcacattcatcacga

SEQ ID NO: 27 - DNA, 20 bp, synthetic construct
  source 1..20: mol_type = other DNA
aacgctgatgggtcacgacg

SEQ ID NO: 28 - DNA, 23 bp, synthetic construct
  source 1..23: mol_type = other DNA
cgcggcaactttgtagtaccagc

SEQ ID NO: 29 - DNA, 25 bp, synthetic construct
  source 1..25: mol_type = other DNA
cccttttcagatttctgcccgacgc

SEQ ID NO: 30 - DNA, 23 bp, synthetic construct
  source 1..23: mol_type = other DNA
cgacagcatcgccagtcactatg

SEQ ID NO: 31 - DNA, 22 bp, synthetic construct
  source 1..22: mol_type = other DNA
caagtagcgaagcgagcaggac

SEQ ID NO: 32 - DNA, 28 bp, synthetic construct
  source 1..28: mol_type = other DNA
agtcatttaataaggccactgttaaacg

SEQ ID NO: 33 - DNA, 34 bp, synthetic construct
  source 1..34: mol_type = other DNA
gttcctataattgaattgatgagacaaactattc

SEQ ID NO: 34 - DNA, 29 bp, synthetic construct
  source 1..29: mol_type = other DNA
gaaaacttagaataattaaattgactctg

SEQ ID NO: 35 - DNA, 25 bp, synthetic construct
  source 1..25: mol_type = other DNA
tttcgcaattagcattatacgacac

SEQ ID NO: 36 - DNA, 25 bp, synthetic construct
  source 1..25: mol_type = other DNA
cgaggaaaatgtcgtaaacttactg

SEQ ID NO: 37 - DNA, 20 bp, synthetic construct
  source 1..20: mol_type = other DNA
gtcaggtagccagaacaccc

SEQ ID NO: 38 - DNA, 20 bp, synthetic construct
  source 1..20: mol_type = other DNA
gccgggatacgttccttctt

SEQ ID NO: 39 - DNA, 20 bp, synthetic construct
  source 1..20: mol_type = other DNA
acgttcgaaaggcgtaccaa

SEQ ID NO: 40 - DNA, 20 bp, synthetic construct
  source 1..20: mol_type = other DNA
tgagtgccattgtagtgcga

SEQ ID NO: 41 - DNA, 20 bp, synthetic construct
  source 1..20: mol_type = other DNA
gcaggctcggttagggtaag

SEQ ID NO: 42 - DNA, 20 bp, synthetic construct
  source 1..20: mol_type = other DNA
ggctaacgtggcaggaatct

SEQ ID NO: 43 - DNA, 20 bp, synthetic construct
  source 1..20: mol_type = other DNA
ttggtaggcctgataagcgc

SEQ ID NO: 44 - DNA, 20 bp, synthetic construct
  source 1..20: mol_type = other DNA
gtagcagatgacctcgcctc

SEQ ID NO: 45 - DNA, 20 bp, synthetic construct
  source 1..20: mol_type = other DNA
tgagtgccagaatcttgcgt

SEQ ID NO: 46 - DNA, 20 bp, synthetic construct
  source 1..20: mol_type = other DNA
acgtacttcgccacctgaag

SEQ ID NO: 47 - DNA, 20 bp, synthetic construct
  source 1..20: mol_type = other DNA
aaggctgggaaatcagacgg

SEQ ID NO: 48 - DNA, 20 bp, synthetic construct
  source 1..20: mol_type = other DNA
tatctgcaaagtcgctgggg

SEQ ID NO: 49 - DNA, 22 bp, synthetic construct
  source 1..22: mol_type = other DNA
gcatgagctcactagtggatcc

SEQ ID NO: 50 - DNA, 33 bp, synthetic construct
  source 1..33: mol_type = other DNA
ctgtcgtcggtgacagattaatgtcattgtgac

SEQ ID NO: 51 - DNA, 30 bp, synthetic construct
  source 1..30: mol_type = other DNA
tgcgttgatgcaatttctatgcgcacccgt

SEQ ID NO: 52 - DNA, 33 bp, synthetic construct
  source 1..33: mol_type = other DNA
acgttcgcgtttgccgtgcgtgtaatgtagtac

SEQ ID NO: 53 - DNA, 35 bp, synthetic construct
  source 1..35: mol_type = other DNA
tcgcaatttagtgtcgttattcgcaaattaatgtc

SEQ ID NO: 54 - DNA, 34 bp, synthetic construct
  source 1..34: mol_type = other DNA
atgtcgtaattcgcaaatttgtgtcgtttttcgc

SEQ ID NO: 55 - DNA, 34 bp, synthetic construct
  source 1..34: mol_type = other DNA
cacacccataaattgatattgcctcttcatggtc

SEQ ID NO: 56 - DNA, 31 bp, synthetic construct
  source 1..31: mol_type = other DNA
tcgttgttaacagattgctgtcgctattaac

SEQ ID NO: 57 - DNA, 61 bp, synthetic construct
  source 1..61: mol_type = other DNA
gactggagttcagacgtgtgctcttccgatcttcataataaattcatctgttgatcgtggg

SEQ ID NO: 58 - DNA, 59 bp, synthetic construct
  source 1..59: mol_type = other DNA
acactctttccctacacgacgctcttccgatctcacaatgacattaatctgtcaccgac

SEQ ID NO: 59 - DNA, 60 bp, synthetic construct
  source 1..60: mol_type = other DNA
acactctttccctacacgacgctcttccgatctccacgacattaatttgcgaataacgac

SEQ ID NO: 60 - DNA, 63 bp, synthetic construct
  source 1..63: mol_type = other DNA
acactctttccctacacgacgctcttccgatctacaaactattctaaacgacattaatttgcg

SEQ ID NO: 61 - DNA, 59 bp, synthetic construct
  source 1..59: mol_type = other DNA
acactctttccctacacgacgctcttccgatcttctacgatacgtagtatctacgatac

SEQ ID NO: 62 - DNA, 57 bp, synthetic construct
  source 1..57: mol_type = other DNA
gactggagttcagacgtgtgctcttccgatcttttcgcaattagcattatacgacac

SEQ ID NO: 63 - DNA, 21 bp, synthetic construct
  source 1..21: mol_type = other DNA
gactggagttcagacgtgtgc

SEQ ID NO: 64 - DNA, 57 bp, synthetic construct
  source 1..57: mol_type = other DNA
acactctttccctacacgacgctcttccgatctgtcaccgacgacagataatttgtc

SEQ ID NO: 65 - DNA, 24 bp, synthetic construct
  source 1..24: mol_type = other DNA
ttcagagcaagagattacgcgcag

SEQ ID NO: 66 - DNA, 26 bp, synthetic construct
  source 1..26: mol_type = other DNA
tgtcgtcttaacaaaataatgtcgtc

SEQ ID NO: 67 - DNA, 24 bp, synthetic construct
  source 1..24: mol_type = other DNA
ttgagtgacacaggaacacttaac

SEQ ID NO: 68 - protein, 639 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRS

SEQ ID NO: 69 - protein, 584 aa, synthetic construct
MNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 70 - protein, 276 aa, synthetic construct
MTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 71 - protein, 167 aa, synthetic construct
MIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 72 - RNA, 230 nt, synthetic construct
  source 1..230: mol_type = other RNA
atattaatagcgccgcaattcatgctgcttgcagcctctgaattttgttaaatgagggttagtttgactgtataaatacagtcttgctttctgaccctggtagctgctcaccctgatgctgctgtcaatagacaggataggtgcgctcccagcaataagggcgcggatgtactgctgtagtggctactgaatcacccccgatcaagggggaaccctaaatgggttgaaag

SEQ ID NO: 73 - protein, 642 aa, synthetic construct
MSVITIQCRLVAEEDSLRQLWELMSEKNTPFINEILLQIGKHPEFETWLEKGRIPAELLKTLGNSLKTQEPFTGQPGRFYTSAITLVDYLYKSWFALQKRRKQQIEGKQRWLKMLKSDQELEQESQSSLEVIRNKATELFSKFTPQSDSEALRRNQNDKQKKVKKTKKSTKPKTSSIFKIFLSTYEEAEEPLTRCALAYLLKNNCQISELDENPEEFTRNKRRKEIEIERLKDQLQSRIPKGRDLTGEEWLETLEIATFNVPQNENEAKAWQAALLRKTANVPFPVAYESNEDMTWLKNDKNRLFVRFNGLGKLTFEIYCDKRHLHYFQRFLEDQEILRNSKRQHSSSLFTLRSGRIAWLPGEEKGEHWKVNQLNFYCSLDTRMLTTEGTQQVVEEKVTAITEILNKTKQKDDLNDKQQAFITRQQSTLARINNPFPRPSKPNYQGKSSILIGVSFGLEKPVTVAVVDVVKNKVIAYRSVKQLLGENYNLLNRQRQQQQRLSHERHKAQKQNAPNSFGESELGQYVDRLLADAIIAIAKKYQAGSIVLPKLRDMREQISSEIQSRAENQCPGYKEGQQKYAKEYRINVHRWSYGRLIESIKSQAAQAGIAIETGKQSIRGSPQEKARDLAVFTYQERQAALI

SEQ ID NO: 74 - protein, 621 aa, synthetic construct
MADEEFEFTEGTTQVPDAILLDKSNFVVDPSQIILATSDRHKLTFNLIQWLAESPNRTIKSQRKQAVANTLDVSTRQVERLLKQYDEDKLRETAGIERADKGKYRVSEYWQNFITTIYEKSLKEKHPISPASIVREVKRHAIVDLELKLGEYPHQATVYRILDPLIEQQKRKTRVRNPGSGSWMTVVTRDGELLRADFSNQIIQCDHTKLDVRIVDNHGNLLSDRPWLTTIVDTFSSCVVGFRLWIKQPGSTEVALALRHAILPKNYPEDYQLNKSWDVCGHPYQYFFTDGGKDFRSKHLKAIGKKLGFQCELRDRPPEGGIVERIFKTINTQVLKELPGYTGANVQERPENAEKEACLTIQDLDKILASFFCDIYNHEPYPKEPRDTRFERWFKGMGGKLPEPLDERELDICLMKEAQRVVQAHGSIQFENLIYRGEFLKAHKGEYVTLRYDPDHILSLYIYSGETDDNAGEFLGYAHAVNMDTHDLSIEELKALNKERSNARKEHFNYDALLALGKRKELVEERKEDKKAKRNSEQKRLRSASKKNSNVIELRKSRTSKSLKKQENQEVLPERISREEIKLEKIEQQPQENLSASPNTQEEERHKLVFSNRQKNLNKIW

SEQ ID NO: 75 - protein, 283 aa, synthetic construct
MAQPQLATQSIVEVLAPRLDIKAQIAKTIDIEEIFRACFITTDRASECFRWLDELRILKQCGRIIGPRNVGKSRAALHYRDEDKKRVSYVKAWSASSSKRLFSQILKDINHAAPTGKRQDLRPRLAGSLELFGLELVIIDNAENLQKEALLDLKQLFEECNVPIVLAGGKELDDLLHDCDLLTNFPTLYEFERLEYDDFKKTLTTIELDVLSLPEASNLAEGNIFEILAVSTEARMGILIKILTKAVLHSLKNGFHRVDESILEKIASRYGTKYIPLKNRNRD

SEQ ID NO: 76 - protein, 184 aa, synthetic construct
MAQNIFLSKTEIGIDEDDEIRPKLGYVEPYEEESISHYLGRLRRFKANSLPSGYSLGKIAGLGAMISRWEKLYFNPFPTLQELEALSSVVGVNADRLIEMLPSQGMTMKPRPIRLCGACYAESPCHRIEWQCKDRMKCDRHNLRLLIKCTNCETPFPIPADWVKGQCPHCSLPFAKMAKRQRRD

SEQ ID NO: 77 - RNA, 270 nt, synthetic construct
  source 1..270: mol_type = other RNA
atatggatacaacagcgccgtagttcatgctccttggagtctctgtactatgaaaaatctggcttagtttggcagttggaagactgtcatgctttctgagcctggtagctgcccgcttctgatgctgctgtcgcaagacaggataggtgcgctcccagcaataaggagtaaggcttttagccatagtcgttatttataacgatgtggatttccacagtggtggctactgaatcacccccttcgtcgggggaaccctaaatgggttgaaag

SEQ ID NO: 78 - protein, 645 aa, synthetic construct
MSTITIQCRLVAEEATLRYFWELMAEKNTPLINELLEQLGQHPDFDTWVQAGKMPEKTVENLCKSLEDREPFANQPGRFRTSAVALVKYIYKSWFALQKRRADRLEGKERWLKMLKSDVELERESNCSLDIIRAKAGEILAKVTEGCAPSNQTSSKRKKKKTKKSQATKDLPTLFEIILKAYEQAEESLTRAALAYLLKNDCEVSEVDEDSEKFKKRRRKKEIEIERLRNQLKSRIPKGRDLTGDKWLKTLEEATRNVPENEDEAKAWQAQLLREASSVPFPVAYETSEDMTWFTNEQGRIFVYFNGSAKHKFQVYCDRRQLHWFQRFVEDFQIKKNGDKKGSEKEYPAGLLTLCSTRLRWKESAEKGDPWNVHRLILSCTIDTRLWTLEGTEQVRAEKIAQVEKTISKREQEVNLSKTQLERLQAKHSERERLNNIFPNRPSKPSYRGKSHIAIGVSFSLENPATVAVVDVATKKVLTYRSFKQLLGDNYNLANRLRQQKQRLSHERHKAQKQGAPNSFGDSELGQYVDRLLAKSIVAIAKTYQASSIVLPKLRYMREIIHNEVQAKAEKKIPGYKEGQKQYAKQYRISVHQWSYNRLSQILESQATKAGISIERGSQVIQGSSQEQARDLALFAYNERQLSLG

SEQ ID NO: 79 - protein, 622 aa, synthetic construct
MGLDEEFEFTEELTQAPDVIVLDKSHFVVDPSQIILQTSDKHKLRFNLIKWFAESPNITIKSQRKQAVVDTLGVSTRQVERLLKQYHNGELSETAGVQRSDKGKLRISQYWEDYIKTTYEKSLKDKHPMLPAAVVREVKRHAIVDLGLKPGDYPHPATIYRNLAPLIEQHTRKKKVRNPGSGSWLTVVTRDGQLLKADFSNQIIQCDHTELDIHIVDSHGSLLSDRPWLTTVVDTYSSCILGFHLWIKQPGSTEVALALRHAILPKNYPEDYKLGKVWEIYGPPFQYFFTDGGKDFNSKHLKAIGKKLGFQCELRNRPPQGGIVERLFKTINTQVLKELPGYTGANVQERPKNAEKEACLTIQDLDKILASFFCDIYNHEPYPKEPRNTRFERWFKGMGGKLPEPLDERELDICLMKEAQRVVQAHGSIQFENLIYRGEALKAYRGEYVTLRYDPDHVLTLYVYSCEADDNAEEFLGYAHAINMDTHDLSIEELKTLNKERSKARSDHYNYDALLALGKRKELVEERKQDKKAKRQSEQKRLRTASKKNSNVIELRKSRASSSSSKDDRQEILPERVSRDELKPEKTELKYEENLLAQTDTQKQERHKLVVSDRKKNLKNIW

SEQ ID NO: 80 - protein, 284 aa, synthetic construct
MAISQLATQPFVEVLPPELDSKAQIAKTIDIEELFRINFITTDRSSECFRWLDELRILKQCGRIIGPRNVGKSRAVLHYRNEDKKRVSYVKAWSASSSKRLFSQILKDINHAASTGKRQDLRPRLAGSLELFGLELVIVDNAENLQKEALLDLKQLFEECHVPIVLVGGKELDDILEDFDLLTNFPTLYEFERLEHDDFIKTLKTIELDILSLPEASKLSEGNIFAILAESTGGKIGILVKILTKAVLHSLKKGFGKVDESILEKIASRYGTKYVPIENKNRND

SEQ ID NO: 81 - protein, 171 aa, synthetic construct
MIEDDEIRLRLGYVEPHPGESISHYLGRLRRFKANSLPSGYALGKIAGLGSVLTRWEKLYFNPFPTQQELEALAQVIQVEVEKLREMLPTKGVTMMPRPIRLCAACYAESPYHRIEWQFKDKMKCDRHQLRLLTKCTNCQTPFPIPADWEKGECSHCFLSFAKMVKCQKRR

SEQ ID NO: 82 - RNA, 351 nt, synthetic construct
  source 1..351: mol_type = other RNA
gggtactaatagcgccgcagttcatgctctttaagagtctctgtactgtggaaaatctgggttagtttgacggttggaaaaccgttttgctttctgaccctggtagctgcccgcttctcatgctctgacttttcacgttatgtggaaaaagtaacgtaatttcgttagttaagacttaccgtaaaaagtcagttctgatgctgctgtcgcaagacaggataggtgcgctcccagcaaaaggagtatgtcttgaaaaagactagccgttctagtaacggtgcggattaccgcagtggtggctactgaatcacccccttcgtcgggggaaccctccaaaaggtgggttgaaag

SEQ ID NO: 83 - protein, 635 aa, synthetic construct
MSVITIQCRLVAEEDILRQLWELMADKNTPLINELLAQVGKHPEFETWLDKGRIPTKLLKTLVNSFKTQERFADQPGRFYTSAIALVDYVYKSWFALQKRRKRQIEGKERWLTILKSDLQLEQESQCSLSAIRTKANEILTQFTPQSEQNKNQRKGKKTKKSTKSEKSSLFQILLNTYEQTQNPLTRCAIAYLLKNNCQISELDEDSEEFTKNRRKKEIEIERLKNQLQSRIPKGRDLTGEEWLKTLEISTANVPQNENEAKAWQAALLRKSADVPFPVAYESNEDMTWLQNDKGRLFVRFNGLGKLTFEIYCDKRHLHYFKRFLEDQELKRNHKNQYSSSLFTLRSGRLAWSPGEEKGEPWKVNQLHLYCTLDTRMWTIEGTQQVVDEKSTKINETLTKAKQKDDLNDQQQAFITRQQSTLDRINNLFPRPSKSRYQGQPSILVGVSFGLKKPVTVAVVDVVKNEVLAYRSVKQLLGENYNLLNRQRQQQQRLSHERHKAQKQNAPNSFGESELGQYIDRLLADAIIAIAKTYQAGSIVLPKLRDMREQISSEIQSRAEKKCPGYKEVQQKYAKEYRMSVHRWSYGRLIECIKSQAAKAGISTEIGTQPIRGSPQEKARDVAVFAYQERQAALI

SEQ ID NO: 84 - protein, 580 aa, synthetic construct
MDEMPIVKQDDESLPVENNDDVDEIQDDELEETNVIFTELSAEAKLKMDVIQGLLEPCDRKTYGEKLRVAAEKLGKTVRTVQRLVKKYQQDGLSAIVETQRNDKGSYRIDPEWQKFIVNTFKEGNKGSKKMTPAQVAMRVQVRAEQLGLQKFPSHMTVYRVLNPIIERQERKQKQRNIGWRGSRVSHKTRDGQTLDVRYSNHVWQCDHTKLDVMLVDQYGEPLARPWFTKITDSYSRCIMGIHVGFDAPSSQVVALASRHAILPKQYSAEYKLISDWGTYGVPENLFTDGGRDFRSEHLKQIGFQLGFECHLRDRPSEGGIEERSFGTINTEFLSGFYGYLGSNIQERSKTAEEEACLTLRELHLLLVRYIVDNYNQRLDARTKDQTRFQRWEAGLPALPKMVKERELDICLMKKTRRSIYKGGYLSFENIMYRGDYLAAYAGENIVLRYDPRDITTVWVYRIDKGKEVFLSAAHALDWETEQLSLEEAKAASRKVRSVGKTLSNKSILAEIHDRDTFIKQKKKSQKERKKEEQAQVHAVYEPINLSETEPLENLQETPKPVTRKPRIFNYEQLRQDYDE

SEQ ID NO: 85 - protein, 279 aa, synthetic construct
MKDDYWQRWVQNLWGDEPIPEELQPEIERLLSPSVVELEHIQKIHDWLDGLRLSKQCGRIVAPPRAGKSVTCDVYRLLNKPQKRGGKRDIVPVLYMQVPGDCSSGELLVLILESLKYDATSGKLTDLRRRVQRLLKESKVEMLIIDEANFLKLNTFSEIARIYDLLRISIVLVGTDGLDNLIKREPYIHDRFIECYKLPLVESEKKFTELVKIWEEEVLCLPLPSNLTRSETLEPLRRKTGGKIGLVDRVLRRASILALRKGLKNIDKETLTEVLDWFE

SEQ ID NO: 86 - protein, 167 aa, synthetic construct
MEIGAEEPHIFEVEPLEGESLSHFLGRFRRENYLTSSQLGKLTGLGAVVSRWKKLYFNPFPTRQELEALTSVVRVNADRLAEMLPPKGVTMKPRPIRLCAACYAEVPCHRIEWQFKDVMKCDRHNLRLLTKCTNCETSFPIPAEWVQGECPHCFLPFATMAKRQKHG

SEQ ID NO: 87 - RNA, 285 nt, synthetic construct
  source 1..285: mol_type = other RNA
atatttttataacagcgccgcagttcatgcttttttaagccaatgtactgtgaaaaatctgggttagtttggcggttggaaggccgtcatgctttctgacccttgtagctgcccgcttctgatgctgccatctttagaattctataggtgggataggtgcgctcccagcaataaggagtaaggcttttagctatagccgttattcataacggtgcggattaccacagtggtggctactgaatcacccccttcgtcgggggaaccctccaaaaggtgggttgaaag

SEQ ID NO: 88 - RNA, 285 nt, synthetic construct
  source 1..285: mol_type = other RNA
atattcttataacagcgccgcagttcatgctttcttaagccaatgtactgtgaaaaatctgggttagtttggcggttggaaggccgtcatgctttctgacccttgtagctgcccgcttctgatgctgccatctttagaattctataggtgggataggtgcgctcccagcaataaggagtaaggcttatagctatagccgttattcataacggtgcggattaccacagtggtggctactgaatcacccccttcgtcgggggaaccctccaaaaggtgggttgaaag

SEQ ID NO: 89 - protein, 255 aa, synthetic construct
MGSDLTYAYLVGLFEGDGYFSITKKGKYLTYELGIELSIKDVQLIYKIKKILGIGIVSFRKRNEIEMVALRIRDKNHLKSFILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNKKLQYLLWLKQLRKISRYSEKIKIPSNY

SEQ ID NO: 90 - protein, 255 aa, synthetic construct
MGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNKKLQYKLWLKQLRKISRYSEKIKIPSNY

SEQ ID NO: 91 - protein, 255 aa, synthetic construct
MGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNY

SEQ ID NO: 92 - protein, 255 aa, synthetic construct
MGSDLTYAYLVGLYEGDGYFSITKKGKYLTYELGIELSIKDVQLIYKIKKILGIGIVSFRKRNEIEMVALRIRDKNHLKSKILPIFEKYPMFSNKQYDYLRFRNALLSGIIYLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNKKLQYKLWLKQLRKISRYSEKIKIPSNY

SEQ ID NO: 93 - protein, 255 aa, synthetic construct
MGSDLTYAYLVGLYEGDGYFSITKKGKYLTYELGIELSIKDVQLIYKIKKILGIGIVSFRKRNEIEMVALRIRDKNHLKSKILPIFEKYPMFSNKQYDYLRFRNALLSGIIYLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNY

SEQ ID NO: 94 - protein, 856 aa, synthetic construct
MGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNYSGSETPGTSESATPESGSNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 95 - protein, 856 aa, synthetic construct
MGSDLTYAYLVGLYEGDGYFSITKKGKYLTYELGIELSIKDVQLIYKIKKILGIGIVSFRKRNEIEMVALRIRDKNHLKSKILPIFEKYPMFSNKQYDYLRFRNALLSGIIYLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNYSGSETPGTSESATPESGSNSQQNPDLAVHPLAIPMEGLLGESATTLEKNVIATQLSEEAQVKLEVIQSLLEPCDRTTYGQKLREAAEKLNVSLRTVQRLVKNWEQDGLVGLTQTSRADKGKHRIGEFWENFITKTYKEGNKGSKRMTPKQVALRVEAKARELKDSKPPNYKTVLRVLAPILEKQQKAKSIRSPGWRGTTLSVKTREGKDLSVDYSNHVWQCDHTRVDVLLVDQHGEILSRPWLTTVIDTYSRCIMGINLGFDAPSSGVVALALRHAILPKRYGSEYKLHCEWGTYGKPEHFYTDGGKDFRSNHLSQIGAQLGFVCHLRDRPSEGGVVERPFKTLNDQLFSTLPGYTGSNVQERPEDAEKDARLTLRELEQLLVRYIVDRYNQSIDARMGDQTRFERWEAGLPTVPVPIPERDLDICLMKQSRRTVQRGGCLQFQNLMYRGEYLAGYAGETVNLRFDPRDITTILVYRQENNQEVFLTRAHAQGLETEQLALDEAEAASRRLRTAGKTISNQSLLQEVVDRDALVATKKSRKERQKLEQTVLRSAAVDESNRESLPSQIVEPDEVESTETVHSQYEDIEVWDYEQLREEYGF

SEQ ID NO: 96 - protein, 893 aa, synthetic construct
MGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNYSGSETPGTSESATPESGSADEEFEFTEGTTQVPDAILLDKSNFVVDPSQIILATSDRHKLTFNLIQWLAESPNRTIKSQRKQAVANTLDVSTRQVERLLKQYDEDKLRETAGIERADKGKYRVSEYWQNFITTIYEKSLKEKHPISPASIVREVKRHAIVDLELKLGEYPHQATVYRILDPLIEQQKRKTRVRNPGSGSWMTVVTRDGELLRADFSNQIIQCDHTKLDVRIVDNHGNLLSDRPWLTTIVDTFSSCVVGFRLWIKQPGSTEVALALRHAILPKNYPEDYQLNKSWDVCGHPYQYFFTDGGKDFRSKHLKAIGKKLGFQCELRDRPPEGGIVERIFKTINTQVLKELPGYTGANVQERPENAEKEACLTIQDLDKILASFFCDIYNHEPYPKEPRDTRFERWFKGMGGKLPEPLDERELDICLMKEAQRVVQAHGSIQFENLIYRGEFLKAHKGEYVTLRYDPDHILSLYIYSGETDDNAGEFLGYAHAVNMDTHDLSIEELKALNKERSNARKEHFNYDALLALGKRKELVEERKEDKKAKRNSEQKRLRSASKKNSNVIELRKSRTSKSLKKQENQEVLPERISREEIKLEKIEQQPQENLSASPNTQEEERHKLVFSNRQKNLNKIW

SEQ ID NO: 97 - protein, 894 aa, synthetic construct
MGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNYSGSETPGTSESATPESGSGLDEEFEFTEELTQAPDVIVLDKSHFVVDPSQIILQTSDKHKLRFNLIKWFAESPNITIKSQRKQAVVDTLGVSTRQVERLLKQYHNGELSETAGVQRSDKGKLRISQYWEDYIKTTYEKSLKDKHPMLPAAVVREVKRHAIVDLGLKPGDYPHPATIYRNLAPLIEQHTRKKKVRNPGSGSWLTVVTRDGQLLKADFSNQIIQCDHTELDIHIVDSHGSLLSDRPWLTTVVDTYSSCILGFHLWIKQPGSTEVALALRHAILPKNYPEDYKLGKVWEIYGPPFQYFFTDGGKDFNSKHLKAIGKKLGFQCELRNRPPQGGIVERLFKTINTQVLKELPGYTGANVQERPKNAEKEACLTIQDLDKILASFFCDIYNHEPYPKEPRNTRFERWFKGMGGKLPEPLDERELDICLMKEAQRVVQAHGSIQFENLIYRGEALKAYRGEYVTLRYDPDHVLTLYVYSCEADDNAEEFLGYAHAINMDTHDLSIEELKTLNKERSKARSDHYNYDALLALGKRKELVEERKQDKKAKRQSEQKRLRTASKKNSNVIELRKSRASSSSSKDDRQEILPERVSRDELKPEKTELKYEENLLAQTDTQKQERHKLVVSDRKKNLKNIW

SEQ ID NO: 98 - protein, 878 aa, synthetic construct
MYPYDVPDYAGGGSGPKKKRKVGGGSGGSDLTYAYLVGLFEGDGYFSITKKGKYLTYELGIELSIKDVQLIYKIKKILGIGIVSFRKRNEIEMVALRIRDKNHLKSKILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNMKLQYKLWLKQLRKISRYSEKIKIPSNYSGSETPGTSESATPESGSDEMPIVKQDDESLPVENNDDVDEIQDDELEETNVIFTELSAEAKLKMDVIQGLLEPCDRKTYGEKLRVAAEKLGKTVRTVQRLVKKYQQDGLSAIVETQRNDKGSYRIDPEWQKFIVNTFKEGNKGSKKMTPAQVAMRVQVRAEQLGLQKFPSHMTVYRVLNPIIERQERKQKQRNIGWRGSRVSHKTRDGQTLDVRYSNHVWQCDHTKLDVMLVDQYGEPLARPWFTKITDSYSRCIMGIHVGFDAPSSQVVALASRHAILPKQYSAEYKLISDWGTYGVPENLFTDGGRDFRSEHLKQIGFQLGFECHLRDRPSEGGIEERSFGTINTEFLSGFYGYLGSNIQERSKTAEEEACLTLRELHLLLVRYIVDNYNQRLDARTKDQTRFQRWEAGLPALPKMVKERELDICLMKKTRRSIYKGGYLSFENIMYRGDYLAAYAGENIVLRYDPRDITTVWVYRIDKGKEVFLSAAHALDWETEQLSLEEAKAASRKVRSVGKTLSNKSILAEIHDRDTFIKQKKKSQKERKKEEQAQVHAVYEPINLSETEPLENLQETPKPVTRKPRIFNYEQLRQDYDE

SEQ ID NO: 99 - protein, 823 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRSSGSETPGTSESATPESGSIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 100 - protein, 1001 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRSSGSETPGTSESATPESGSIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKVGGGSGGGSGGGSIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 101 - protein, 932 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRSSGSETPGTSESATPESGSTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 102 - protein, 1110 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRSSGSETPGTSESATPESGSIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKVGGGSGGGSGGGSTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYK

SEQ ID NO: 103 - protein, 1110 aa, synthetic construct
MSQITIQARLISFESNRQQLWKLMADLNTPLINELLCQLGQHPDFEKWQQKGKLPSTVVSQLCQPLKTDPRFAGQPSRLYMSAIHIVDYIYKSWLAIQKRLQQQLDGKTRWLEMLNSDAELVELSGDTLEAIRVKAAEILAIAMPASESDSASPKGKKGKKEKKPSSSSPKRSLSKTLFDAYQETEDIKSRSAISYLLKNGCKLTDKEEDSEKFAKRRRQVEIQIQRLTEKLISRMPKGRDLTNAKWLETLLTATTTVAEDNAQAKRWQDILLTRSSSLPFPLVFETNEDMVWSKNQKGRLCVHFNGLSDLIFEVYCGNRQLHWFQRFLEDQQTKRKSKNQHSSGLFTLRNGHLVWLEGEGKGEPWNLHHLTLYCCVDNRLWTEEGTEIVRQEKADEITKFITNMKKKSDLSDTQQALIQRKQSTLTRINNSFERPSQPLYQGQSHILVGVSLGLEKPATVAVVDAIANKVLAYRSIKQLLGDNYELLNRQRRQQQYLSHERHKAQKNFSPNQFGASELGQHIDRLLAKAIVALARTYKAGSIVLPKLGDMREVVQSEIQAIAEQKFPGYIEGQQKYAKQYRVNVHRWSYGRLIQSIQSKAAQTGIVIEEGKQPIRGSPHDKAKELALSAYNLRLTRRSSGSETPGTSESATPESGSTEAQAIAKQLGGVKPDDEWLQAEIARLKGKSIVPLQQVKTLHDWLDGKRKARKSCRVVGESRTGKTVACDAYRYRHKPQQEAGRPPTVPVVYIRPHQKCGPKDLFKKITEYLKYRVTKGTVSDFRDRTIEVLKGCGVEMLIIDEADRLKPETFADVRDIAEDLGIAVVLVGTDRLDAVIKRDEQVLERFRAHLRFGKLSGEDFKNTVEMWEQMVLKLPVSSNLKSKEMLRILTSATEGYIGRLDEILREAAIRSLSRGLKKIDKAVLQEVAKEYKGGGSGGGSGGGSIEAPDVKPWLFLIKPYEGESLSHFLGRFRRANHLSASGLGTLAGIGAIVARWERFHFNPRPSQQELEAIASVVEVDAQRLAQMLPPAGVGMQHEPIRLCGACYAESPCHRIEWQYKSVWKCDRHQLKILAKCPNCQAPFKMPALWEDGCCHRCRMPFAEMAKLQKV

SEQ ID NO: 104 - DNA, 472 bp, synthetic construct
  source 1..472: mol_type = other DNA
ttagacatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagcaatgctacctc

SEQ ID NO: 105 - DNA, 462 bp, synthetic construct
  source 1..462: mol_type = other DNA
cgagtctcctattctccattatatatgtacattcgcaaattaaatgtcgcttttcgcaatttagtgtcgttattcgcaaattaatgtcgtggtggttgtttttcagagtcaatttaattattctaagttttcgcaaattaatgtcgcatgaacttaacatttactatacaataaattattgctgcaagggcattattggattattgatatgtgttcgatcgcagcactcctgacatctaatttgcaaaataccaaattcttaacaaacgacatttaatttgcgaaaccaggttttacgacatacaatatgcgaattaggtaacttagtcttttgtaggggtaaatagcttatgatgcttatagaataaaggttttagtccttaaaagcagttgcgacactaatttgcgaaaagcgacatttaatttgcgaacgtacaatagcctttctcactctagttagat

SEQ ID NO: 106 - DNA, 459 bp, synthetic construct
  source 1..459: mol_type = other DNA
ttagacatctccacaaaaggcgtagtgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacggacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtacacgtagagacgtagcaatgctacctc

SEQ ID NO: 107 - DNA, 463 bp, synthetic construct
  source 1..463: mol_type = other DNA
ttagacatctccacaaaaggcgtagtgtacattcgcaaattaaatgtcgcttttcgcaatttagtgtcgttattcgcaaattaatgtcgtggtggttgtttttcagagtcaatttaattattctaagttttcgcaaattaatgtcgcatgaacttaacatttactatacaataaattattgctgcaagggcattattggattattgatatgtgttcgatcgcagcactcctgacatctaatttgcaaaataccaaattcttaacaaacgacatttaatttgcgaaaccaggttttacgacatacaatatgcgaattaggtaacttagtcttttgtaggggtaaatagcttatgatgcttatagaataaaggttttagtccttaaaagcagttgcgacactaatttgcgaaaagcgacatttaatttgcgaacgtacacgtagagacgtagctaatgctacctc

SEQ ID NO: 108 - DNA, 850 bp, synthetic construct
  source 1..850: mol_type = other DNA
aaatccagctgctggctttaacttatgtcgaataactaattatttgtcgttgttaacagattgctgtcgctattaacaaattaatgtcactgttaacaaattagtgtcgtataatgctaattgcgaaacgttaacaaattaatgtcgtctaaccaatttgataaagtgtttgcagacatctattgtacaggaaatatagctaaatctttatttgatgacttccctgataatattcataaatatgcttacaagtcggatgcacctttcaaccctctgttaaatattttctgacgctctttcaactcatccctagctgggatagttgttgaaacttagagtcacccagtttggcattagatactatcttttttcaacctacccctaaccaggatggtcgttgaaacctggatatgctcaatacaaggaaaacttgattcatactcaaaacagtaatcacaatctcgctattgtgcgagaacatccaaacttcctaaagcagttgacccctcaatggacgcggcaacttttcggtataaggatgtattatttagtgcaaatgtactaaataaaattataataccactattcaagctaaaaagcgacagctaatttgttatgaaactagaaaattttagaaaacgtaaaattttaaaagacgacgtttattttgttattatttaaatcaacgacaagtaaagtgttaaataaactactaacccattacataataaaaaacgttgtaaacactcatgtagcaacatttttgatagttttatatttgacgacattattttgttaagacgacaaataattagttattcaacaacttaaatttatctgcatttaattg

SEQ ID NO: 109 - protein, 89 aa, synthetic construct
MSLSTEATAKIVSEFGRDANDTGSTEVQVALLTAQINHLQGHFAEHKKDHHSRRGLLRMVSQRRKLLDYLKRKDVARYTQLIERLGLRR

SEQ ID NO: 110 - protein, 89 aa, synthetic construct
MALTQQRKQEIITNFQVHETDTGSADVQIAMLTERINRLSEHLQANKKDHSSRRGLLKLIGHRKRLLAYLQQESREKYQALIARLGIRG

SEQ ID NO: 111 - protein, 89 aa, synthetic construct
MALTQQRKQELISGYQVHETDTGSADVQIAMLTDRINRLSQHLQANKKDHSSRRGLLKMIGQRKRLLSYIQKGSREKYQALIARLGIRG

SEQ ID NO: 112 - protein, 89 aa, synthetic construct
MALTQERKQEIIVNYQVHETDTGSADVQVAMLTERINRLSLHLQANKKDHSSRRGLLKLIGQRKRLLAYIQKDSREKYQALIGRLGIRG

SEQ ID NO: 113 - protein, 305 aa, synthetic construct
MRLKVMMDVNKKTKIRHRNELNHTLAQLPLPAKRVMYMALAPIDSKEPLERGRVFKIRAEDLAALAKITPSLAYRQLKEGGKLLGASKISLRGDDIIALAKELNLPFTAKNSPEELDLNIIEWIAYSNDEGYLSLKFTRTIEPYISSLIGKKNKFTTQLLTASLRLSSQYSSSLYQLIRKHYSNFKKKNYFIISVDELKEELIAYTFDKDGNIEYKYPDFPIFKRDVLNKAIAEIKKKTEISFVGFTVHEKEGRKISKLKFEFVVDEDEFSGDKDDEAFFMNLSEADAAFLKVFDETVPPKKAKG

SEQ ID NO: 114 - protein, 90 aa, synthetic construct
MNKTQLIDVIAEKAELSKTQAKAALESTLAAITESLKEGDAVQLVGFGTFKVNHRAERTGRNPQTGKEIKIAAANVPAFVSGKALKDAVK

SEQ ID NO: 115 - protein, 90 aa, synthetic construct
MNKSQLIDKIAAGADISKAAAGRALDAIIASVTESLKEGDDVALVGFGTFAVKERAARTGRNPQTGKEITIAAAKVPSFRAGKALKDAVN

SEQ ID NO: 116 - protein, 200 aa, synthetic construct
NKTQLIDVIAEKAELSKTQAKAALESTLAAITESLKEGDAVQLVGFGTFKVNHRAERTGRNPQTGKEIKIAAANVPAFVSGKALKDAVKSGSGSETPGTSESATPESGSGSNKSQLIDKIAAGADISKAAAGRALDAIIASVTESLKEGDDVALVGFGTFAVKERAARTGRNPQTGKEITIAAAKVPSFRAGKALKDAVN

SEQ ID NO: 117 - protein, 94 aa, synthetic construct
MNKGELVDAVAEKASVTKKQADAVLTAALETIIEAVSSGDKVTLVGFGSFESRERKAREGRNPKTNEKMEIPATKVPAFSAGKLFRERVAPPKS

SEQ ID NO: 118 - protein, 94 aa, synthetic construct
MNKGELVDAVAEKASVTKKQADAVLSAALETIIEAVSSGDKVTLVGFGSFESRERKAREGRNPKTNEKMEIPATKVPAFSAGKMFRERVAPPKE

SEQ ID NO: 119 - protein, 94 aa, synthetic construct
MNKGELVDAVAEKASVTKKQADAVLSAALETIIEAVSSGDKVTLVGFGSFESRERKAREGRNPKTNEKMEIPATKVPAFSAGKMFRERVAPPKV

SEQ ID NO: 120 - protein, 99 aa, synthetic construct
MALTKAEMSEYLFDKLGLSKRDAKELVELFFEEIRRALENGEQVKLSGFGNFDLRDKNQRPGRNPKTGEDIPITARRVVTFRPGQKLKSRVENASPKDE

SEQ ID NO: 121 - protein, 94 aa, synthetic construct
MTKSELIERLATQQSHIPAKTVEDAVKEMLEHMASTLAQGERIEIRGFGSFSLHYRAPRTGRNPKTGDKVELEGKYVPHFKPGKELRDRANIYG

SEQ ID NO: 122 - protein, 214 aa, synthetic construct
MTKSELIERLATQQSHIPAKTVEDAVKEMLEHMASTLAQGERIEIRGFGSFSLHYRAPRTGRNPKTGDKVELEGKYVPHFKPGKELRDRANIYGSGSGSETPGTSESATPESGSGSALTKAEMSEYLFDKLGLSKRDAKELVELFFEEIRRALENGEQVKLSGFGNFDLRDKNQRPGRNPKTGEDIPITARRVVTFRPGQKLKSRVENASPKDE

SEQ ID NO: 123 - protein, 214 aa, synthetic construct
MALTKAEMSEYLFDKLGLSKRDAKELVELFFEEIRRALENGEQVKLSGFGNFDLRDKNQRPGRNPKTGEDIPITARRVVTFRPGQKLKSRVENASPKDESGSGSETPGTSESATPESGSGSTKSELIERLATQQSHIPAKTVEDAVKEMLEHMASTLAQGERIEIRGFGSFSLHYRAPRTGRNPKTGDKVELEGKYVPHFKPGKELRDRANIYG

SEQ ID NO: 124 - RNA, 12 nt, synthetic construct
  source 1..12: mol_type = other RNA
gggaatcctccc

SEQ ID NO: 125 - RNA, 14 nt, synthetic construct
  source 1..14: mol_type = other RNA
ggggtgggttgaag

SEQ ID NO: 126 - RNA, 26 nt, synthetic construct
  source 1..26: mol_type = other RNA
gggaatcctccctaaatgggtgggtt

SEQ ID NO: 127 - RNA, 22 nt, synthetic construct
  source 1..22: mol_type = other RNA
gggaaccctaaatgggttggtt

SEQ ID NO: 128 - protein, 4 aa, synthetic construct
GGGS

SEQ ID NO: 129 - DNA, 108 bp, synthetic construct
  source 1..108: mol_type = other DNA
gaggttgagaagtcatttaataaggccactgttaaacgtagatatatctacgcgtagatatatctatgtcgaataactaataattagttattcaacatatctacgata

SEQ ID NO: 130 - RNA, 19 nt, synthetic construct
  source 1..19: mol_type = other RNA
atttttattttttctttta

SEQ ID NO: 131 - RNA, 19 nt, synthetic construct
  source 1..19: mol_type = other RNA
attcttatttcttcttata

SEQ ID NO: 132 - RNA, 26 nt, synthetic construct
  source 1..26: mol_type = other RNA
acgcgtagatatatctatgtcgaata

SEQ ID NO: 133 - DNA, 40 bp, synthetic construct
  source 1..40: mol_type = other DNA
tatatctacgtgtacagtgatcactgtacactacgatacg

SEQ ID NO: 134 - DNA, 33 bp, synthetic construct
  source 1..33: mol_type = other DNA
tgaggaggtttctctgtaannnnnnnnnnnnnn

SEQ ID NO: 135 - DNA, 33 bp, synthetic construct
  source 1..33: mol_type = other DNA
nnnnnnnnnnnnnnttacagagaaacctcctca

SEQ ID NO: 136 - DNA, 33 bp, synthetic construct
  source 1..33: mol_type = other DNA
actcctccaaagagacattnnnnnnnnnnnnnn

SEQ ID NO: 137 - DNA, 33 bp, synthetic construct
  source 1..33: mol_type = other DNA
nnnnnnnnnnnnnnaatgtctctttggaggagt

SEQ ID NO: 138 - DNA, 512 bp, synthetic construct
  source 1..512: mol_type = other DNA
ttagacatctccacaaaaggctttacagagaaacctcctcagtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtatgaggaggtttctctgtaaagagacgtagcaatgctacctc

SEQ ID NO: 139 - DNA, 512 bp, synthetic construct
  source 1..512: mol_type = other DNA
ttagacatctccacaaaagtttacagagaaacctcctcagcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagatgaggaggtttctctgtaaagacgtagcaatgctacctc

SEQ ID NO: 140 - DNA, 512 bp, synthetic construct
  source 1..512: mol_type = other DNA
ttagacatctccacaaatttacagagaaacctcctcaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagatgaggaggtttctctgtaaacgtagcaatgctacctc

SEQ ID NO: 141 - DNA, 512 bp, synthetic construct
  source 1..512: mol_type = other DNA
ttagacatctccacatttacagagaaacctcctcaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtgaggaggtttctctgtaaatagcaatgctacctc

SEQ ID NO: 142 - DNA, 512 bp, synthetic construct
  source 1..512: mol_type = other DNA
ttagacatctccatttacagagaaacctcctcacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtatgaggaggtttctctgtaaagcaatgctacctc

SEQ ID NO: 143 - DNA, 512 bp, synthetic construct
  source 1..512: mol_type = other DNA
ttagacatctctttacagagaaacctcctcacacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagctgaggaggtttctctgtaaaaatgctacctc

SEQ ID NO: 144 - DNA, 512 bp, synthetic construct
  source 1..512: mol_type = other DNA
ttagacatctttacagagaaacctcctcatccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagcaatgaggaggtttctctgtaaatgctacctc

SEQ ID NO: 145 - DNA, 512 bp, synthetic construct
  source 1..512: mol_type = other DNA
ttagacatttacagagaaacctcctcatctccacaaaaggcgtagtgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactgagcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtacacgtagagacgtagcaatgtgaggaggtttctctgtaaactacctc

SEQ ID NO: 146 - DNA, 502 bp, synthetic construct
  source 1..502: mol_type = other DNA
ttagacatctctttacagagaaacctcctcacacaaaaggcgtagtgtacattcgcaaattaaatgtcgcttttcgcaatttagtgtcgttattcgcaaattaatgtcgtggtggttgtttttcagagtcaatttaattattctaagttttcgcaaattaatgtcgcatgaacttaacatttactatacaataaattattgctgcaagggcattattggattattgatatgtgttcgatcgcagcactcctgacatctaatttgcaaaataccaaattcttaacaaacgacatttaatttgcgaaaccaggttttacgacatacaatatgcgaattaggtaacttagtcttttgtaggggtaaatagcttatgatgcttatagaataaaggttttagtccttaaaagcagttgcgacactaatttgcgaaaagcgacatttaatttgcgaacgtacacgtagagacgtagctgaggaggtttctctgtaaaaatgctacctc

SEQ ID NO: 147 - DNA, 499 bp, synthetic construct
  source 1..499: mol_type = other DNA
ttagacatctctttacagagaaacctcctcacacaaaaggcgtagtgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacggacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtacacgtagagacgtagctgaggaggtttctctgtaaaaatgctacctc

SEQ ID NO: 148 - DNA, 890 bp, synthetic construct
  source 1..890: mol_type = other DNA
ttagacatctctttacagagaaacctcctcacacaaaaggcgtagtgtcgaataactaattatttgtcgttgttaacagattgctgtcgctattaacaaattaatgtcactgttaacaaattagtgtcgtataatgctaattgcgaaacgttaacaaattaatgtcgtctaaccaatttgataaagtgtttgcagacatctattgtacaggaaatatagctaaatctttatttgatgacttccctgataatattcataaatatgcttacaagtcggatgcacctttcaaccctctgttaaatattttctgacgctctttcaactcatccctagctgggatagttgttgaaacttagagtcacccagtttggcattagatactatcttttttcaacctacccctaaccaggatggtcgttgaaacctggatatgctcaatacaaggaaaacttgattcatactcaaaacagtaatcacaatctcgctattgtgcgagaacatccaaacttcctaaagcagttgacccctcaatggacgcggcaacttttcggtataaggatgtattatttagtgcaaatgtactaaataaaattataataccactattcaagctaaaaagcgacagctaatttgttatgaaactagaaaattttagaaaacgtaaaattttaaaagacgacgtttattttgttattatttaaatcaacgacaagtaaagtgttaaataaactactaacccattacataataaaaaacgttgtaaacactcatgtagcaacatttttgatagttttatatttgacgacattattttgttaagacgacaaataattagttattcaacacgtagagacgtagctgaggaggtttctctgtaaaaatgctacctc

SEQ ID NO: 149 - DNA, 210 bp, synthetic construct
  source 1..210: mol_type = other DNA
tgtacagtgacaaattatctgtcgtcggtgacagattaatgtcattgtgactatttaattgtcgtcgtgacccatcagcgttgcttaattaattgatgacaaattaaatgtcatcaatataatatgctctgcaattattatacaaagcaattaaaacaagcggataaaaggacttgctttcaacccacccctaagtttaatagttactga

SEQ ID NO: 150 - DNA, 212 bp, synthetic construct
  source 1..212: mol_type = other DNA
gcgacagtcaatttgtcattatgaaaatacacaaaagctttttcctatcttgcaaagcgacagctaatttgtcacaatcacggacaacgacatctattttgtcactgcaaagaggttatgctaaaactgccaaagcgctataatctatactgtataaggattttactgatgacaataatttgtcacaacgacatataattagtcactgtaca

SEQ ID NO: 151 - DNA, 206 bp, synthetic construct
  source 1..206: mol_type = other DNA
tgtacattcgcaaattaaatgtcgcttttcgcaatttagtgtcgttattcgcaaattaatgtcgtggtggttgtttttcagagtcaatttaattattctaagttttcgcaaattaatgtcgcatgaacttaacatttactatacaataaattattgctgcaagggcattattggattattgatatgtgttcgatcgcagcactcct

SEQ ID NO: 152 - DNA, 206 bp, synthetic construct
  source 1..206: mol_type = other DNA
gacatctaatttgcaaaataccaaattcttaacaaacgacatttaatttgcgaaaccaggttttacgacatacaatatgcgaattaggtaacttagtcttttgtaggggtaaatagcttatgatgcttatagaataaaggttttagtccttaaaagcagttgcgacactaatttgcgaaaagcgacatttaatttgcgaacgtaca

SEQ ID NO: 153 - DNA, 203 bp, synthetic construct
  source 1..203: mol_type = other DNA
tgtacattcgcaaattaaatgtcgtaattcgcaaatttgtgtcgtttttcgcaaattaatgtcgtttagaatagtttgtctcatcaattcaattataggaacttttcgcaaattaatgtcgtcctgtttctccatttagtgtcgattaacaaattaatgtcgctgttaacgaattaatgtcgtcgaattagttccaactaacg

SEQ ID NO: 154 - DNA, 206 bp, synthetic construct
  source 1..206: mol_type = other DNA
gacatctaatttgcgaaacaggcaaatcttaataaacgacatttaatttgcgaaaataggatttgcgacatctaatttgcgaaacaggcaaattactcagttttatggataaatagcttgtaagtcctacgcaataaagatctcagctattagaagtaattgcgacactaatttgcgaattgcgacatataatttgcgaatgtaca

SEQ ID NO: 155 - DNA, 400 bp, synthetic construct
  source 1..400: mol_type = other DNA
tgtcgaataactaattatttgtcgttgttaacagattgctgtcgctattaacaaattaatgtcactgttaacaaattagtgtcgtataatgctaattgcgaaacgttaacaaattaatgtcgtctaaccaatttgataaagtgtttgcagacatctattgtacaggaaatatagctaaatctttatttgatgacttccctgataatattcataaatatgcttacaagtcggatgcacctttcaaccctctgttaaatattttctgacgctctttcaactcatccctagctgggatagttgttgaaacttagagtcacccagtttggcattagatactatcttttttcaacctacccctaaccaggatggtcgttgaaacctggatatgctcaatacaagg

SEQ ID NO: 156 - DNA, 400 bp, synthetic construct
  source 1..400: mol_type = other DNA
aaaacttgattcatactcaaaacagtaatcacaatctcgctattgtgcgagaacatccaaacttcctaaagcagttgacccctcaatggacgcggcaacttttcggtataaggatgtattatttagtgcaaatgtactaaataaaattataataccactattcaagctaaaaagcgacagctaatttgttatgaaactagaaaattttagaaaacgtaaaattttaaaagacgacgtttattttgttattatttaaatcaacgacaagtaaagtgttaaataaactactaacccattacataataaaaaacgttgtaaacactcatgtagcaacatttttgatagttttatatttgacgacattattttgttaagacgacaaataattagttattcaaca

SEQ ID NO: 157 - protein, 12 aa, synthetic construct
GGGSGGGSGGGS

(19) United States
(12) Patent Application Publication
(10) Pub. No.: US 2025/0122535 A1
(43) Pub. Date: Apr. 17, 2025

(54) CRISPR-ASSOCIATED TRANSPOSASES AND METHODS OF USE THEREOF

(71) Applicant: The General Hospital Corporation, Boston, MA (US)

(72) Inventors: Benjamin Kleinstiver, Boston, MA (US); Connor J. Tou, Boston, MA (US)

(21) Appl. No.: 18/715,209

(22) PCT Filed: Dec. 2, 2022
(86) PCT No.: PCT/US2022/051639

§ 371 Date: May 31, 2024

Related U.S. Application Data
(60) Provisional application No. 63/411,735, filed on Sep. 30, 2022.
(60) Provisional application No. 63/291,264, filed on Dec. 17, 2021.
(60) Provisional application No. 63/285,857, filed on Dec. 3, 2021.

(51) Int. Cl. C12N 15/90; C12N 9/12; C12N 9/22; C12N 15/11

(52) U.S. Cl. CPC C12N 15/907; C12N 9/1241; C12N 9/22; C12N 15/11; C12N 2310/20; C12N 2800/90

(57) ABSTRACT

Described herein are improved CRISPR-associated transposases (CASTs), including homing endonuclease-assisted large-sequence integrating CRISPR-associated transposases (CAST) complexes and methods of use thereof, and other strategies to improve the activities of natural and engineered CASTs.

### CROSS-REFERENCE TO RELATED APPLICATIONS

This application is the U.S. National Phase application under 35 U.S.C. § 371 of International Patent Application No. PCT/US2022/051639, filed on Dec. 2, 2022, which claims the benefit of U.S. Provisional Patent Application Nos. 63/285,857, filed on Dec. 3, 2021, 63/291,264, filed on Dec. 17, 2021, and 63/411,735, filed on Sep. 30, 2022, the entire contents of each of the foregoing are hereby incorporated by reference in their entireties.

### SEQUENCE LISTING

This application contains a Sequence Listing that has been submitted electronically as an XML file named 29539-0632US1_SL_ST26. The XML file, created on Dec. 12, 2024, is 172,358 bytes in size. The material in the XML file is hereby incorporated by reference in its entirety.

### TECHNICAL FIELD

Described herein are improved CRISPR-associated transposases (CASTs), including homing endonuclease-assisted large-sequence integrating CAST and methods of use thereof.

### BACKGROUND

Programmable insertion of multi-kilobase DNA sequences into genomes without reliance on homologous recombination and double stranded breaks (DSBs) would offer new capabilities for precision genome editing. Methods for genomic integration typically rely on viral vectors^{1,2} or transposons³⁻⁷, both of which lack programmability and thus insert stochastically throughout the genome, or nucleases coupled with DNA donors⁸⁻¹⁰ that rely on cytotoxic DSBs and host homologous recombination factors. Additionally, recombineering systems in bacteria are low efficiency¹¹ without cointegration of a selectable marker¹² or CRISPR-Cas counterselection¹³. CRISPR-associated transposases (CASTs) are a promising new approach for programmable, recombination-independent DNA insertions through an interplay between transposase proteins and CRISPR-Cas effector(s) to direct RNA-guided transposition¹⁴⁻¹⁶.

### SUMMARY

CRISPR-associated transposases (CASTs) enable recombination-independent, multi-kilobase DNA insertions at RNA-programmed genomic locations. Type V-K CASTs offer distinct technological advantages over type I CASTs given their smaller coding size, fewer components, and unidirectional insertions. However, the utility of type V-K CASTs is hindered by high off-target integration and a replicative transposition mechanism that results in a mixture of desired simple cargo insertions and undesired plasmid cointegrate products. Here, we overcome both limitations by engineering new CASTs with improved integration product purity and genome-wide specificity. To do so, we compensate for the absence of the TnsA subunit in type V-K CASTs by engineering a Homing Endonuclease-assisted Large-sequence Integrating CAST-compleX (HELIX), which utilizes a nicking homing endonuclease (nHE) fused to TnsB to restore the 5′ nicking capability needed for cargo excision on the DNA donor. HELIX enables cut-and-paste DNA insertion with up to 99.4% simple insertion product purity, while retaining robust integration efficiencies on genomic targets. We generate and characterize functional fusions between CAST subunits and demonstrate that HELIX has substantially higher on-target specificity compared to canonical CASTs. Further, we identify fusion proteins and a host factor that enhance on-target specificity of HELIX, reducing off-target integration profiles to levels comparable to those of type I systems. We also demonstrate the extensibility of HELIX to other type V-K orthologs as well as the feasibility of CAST- and HELIX-mediated DNA insertion in human cell lysates and human cells. By leveraging distinct features of both type V-K and type I systems, HELIX streamlines and improves the application of CRISPR-based transposition technologies, eliminating barriers for efficient and specific RNA-guided DNA insertions.

Accordingly, provided herein are fusion proteins comprising a transposition protein B (TnsB) protein, e.g., Tn7, Tn7-like, or Tn5053-like transposition protein B (TnsB), fused (optionally via an intervening linker) to a protein (e.g., an endonuclease, e.g., a nickase, cleavase, or catalytically dead endonuclease, a fluorescent protein, or a peptide tag (e.g. NLS, His, Flag)). In some embodiments, the endonuclease is a nickase, e.g., a homing endonuclease (HE), nicking restriction endonuclease, a nicking Cas variant, or a phage HNH endonuclease, or TnsA from a type I CAST or a Tn7 transposon, or a catalytic portion thereof. In some embodiments, the HE is a LAGLIDADG, H—N—H, His-Cys box, or GIY-YIG HE. In some embodiments, the HE is I-AniI, e.g., I-AniI from Aspergillus nidulans (I-AniI) or a variant thereof, optionally comprising a K227M mutation (nAniI), a hyperactive variant (e.g., Y2 I-AniI (F13Y, S111Y)), or both (K227M, F13Y, S111Y). Also provided in some embodiments, are a nucleic acid comprising a sequence encoding the fusion protein as described. Also provided is an expression construct comprising the nucleic acid as described, and regulatory sequences to express the protein, e.g., a promoter.

In some embodiments, provided are expression constructs comprising sequences encoding a CRISPR-associated transposase (CAST), wherein the sequences comprise nucleic acids encoding the fusion protein as described, Cas12k; TnsC; TniQ; optionally one or more host proteins; and a guide RNA (gRNA) that interacts with Cas12k and directs the Cas12k/gRNA complex to a target sequence, and regulatory sequences to express the sequences, e.g., one or more promoter sequences. In some embodiments, the Cas12k is fused to at least one other protein, optionally TniQ and/or TnsC (e.g., Cas12k-TniQ, Cas12k-TniQ-TniQ, Cas12k-TnsC, Cas12k-TniQ-TnsC, or Cas12k-TnsC-TniQ), optionally with a linker in between each protein. In some embodiments, the expression construct is a plasmid or viral vector.

Also provided, in some embodiments, are host cells comprising and optionally expressing the nucleic acid as described comprising nucleic acid sequences encoding a Tn-endonuclease fusion protein, e.g., a TnsB-endonuclease fusion protein; and optionally one or more, e.g., all, of Cas12k; TnsC; TniQ; optionally one or more host proteins; and a guide RNA that binds to cas12k and directs the TnsB-endonuclease fusion protein to a selected target sequence, or a host cell comprising a CRISPR-associated transposase (CAST) comprising the fusion protein as described; Cas12k; TnsC; TniQ; optionally one or more host proteins; and a gRNA that interacts with Cas12k and directs the fusion protein to a selected target sequence. In some embodiments, the Cas12k is fused to at least one other protein, optionally TniQ (e.g., Cas12k-TniQ, TniQ-Cas12k, TniQ-TniQ-Cas12k, TniQ-Cas12k-TniQ, or Cas12k-TniQ-TniQ) and/or at least one TnsC, optionally with a linker in between each protein.

Also provided are methods of inserting a desired sequence into DNA, e.g., into genomic DNA of a living cell, the method comprising expressing in the cell the nucleic acid of claim 5; Cas12k; TnsC; TniQ; optionally one or more host proteins; and a guide RNA that binds to cas12k and directs the endonuclease a selected target sequence, and a donor DNA molecule (e.g. a plasmid) comprising the desired sequence to be inserted, wherein the desired sequence is flanked by LE and RE flanking sequences on the 5′ and 3′ ends, respectively, and a target site for the endonuclease (e.g., I-AniI), preferably wherein the target site is oriented to confer a nick on the donor plasmid 5′ of the desired sequence to be inserted. In some embodiments, the donor DNA molecule has modified LE/RE flanking sequences, e.g., a flanking sequence as shown in Table A that is from a source organism other than the source organism of at least one of the CAST components, i.e., TnsB; cas12k; TnsC; or TniQ, and/or comprising modifications or insertions at varying distances from the LE and RE sequences (e.g. an endonuclease recognition sequence or host factor binding sequence(s)). In some embodiments, the modified LE/RE flanking sequences are from Scytonema hofmannii (e.g., from ShCAST), and wherein at least one of the Tn protein; cas12k; TnsC; or TniQ is from a CAST or HELIX ortholog (e.g. AcCAST and AcHELIX); are modified ShCAST LE/RE flanking sequences; or are de-novo LE/RE flanking sequences. In some embodiments, the Cas12k is expressed as a fusion protein, optionally with at least one TniQ and/or at least one TnsC (e.g., Cas12k-TniQ, Cas12k-TniQ-TniQ, Cas12k-TnsC, Cas12k-TniQ-TnsC, or Cas12k-TnsC-TniQ), optionally with a linker in between each protein.

Also provided are fusion proteins comprising: Cas12k; optionally one or morehost proteins; and at least one TniQ (e.g., Cas12k-TniQ or Cas12k-TniQ-TniQ) and/or at least one TnsC, optionally with a linker in between each segment.

Also provided are fusion proteins comprising a host protein and one or more of Cas12k, TnsC, or TniQ, optionally with a linker in between each segment.

Also provided are compositions comprising, or nucleic acids encoding: (i) a fusion protein comprising a transposon (Tn) protein, e.g., Tn7, Tn7-like, or Tn5053-like, e.g., transposition protein B (TnsB), fused to a protein (e.g., an endonuclease, e.g., a nickase, cleavase, or catalytically dead endonuclease, a fluorescent protein, or a peptide tag (e.g. NLS, His, Flag)), optionally via an intervening linker; and (ii) a fusion protein comprising a host protein and one or more of Cas12k, TnsC, or TniQ, optionally with a linker in between each segment.

Also provided are compositions comprising, or nucleic acids encoding: (ii) a fusion protein comprising a transposon (Tn) protein, e.g., Tn7, Tn7-like, or Tn5053-like, e.g., transposition protein B (TnsB), fused to a protein (e.g., an endonuclease, e.g., a nickase, cleavase, or catalytically dead endonuclease, a fluorescent protein, or a peptide tag (e.g. NLS, His, Flag)), optionally via an intervening linker; and (ii) a fusion protein comprising a host protein and one or more of Cas12k, TnsC, or TniQ, optionally with a linker in between each segment.

In some embodiments, the host factor is ribosomal protein S15, alters DNA topology (e.g., pi protein or a nucleoid-associated protein (NAP), such as, HU, Fis, H—NS, IHF, or TF1) or wherein the host factor is involved in DNA or cellular metabolism, proteolysis or protein folding, regulation, or transport (e.g., acyl carrier protein (ACP), Sigma S, DnaN, DnaA, DNA topoisomerase I, La protease, Dam methylase, or proteins expressed from the genes dcd, dinD, radA, recQ, clpX, fkpA, hflX, crl, rseB, rsxE, araJ, melB, mgtA, aspA, treC, proY, serA, yhbC, yidA, ykfA).

Also provided are host cells comprising or expressing the composition of any one of claims 18-20, and a donor DNA molecule (e.g. a plasmid) comprising the desired sequence to be inserted, wherein the desired sequence is flanked by LE and RE flanking sequences on the 5′ and 3′ ends, respectively, and a target site for the endonuclease (e.g., I-AniI), preferably wherein the target site is oriented to confer a nick on the donor plasmid 5′ of the desired sequence to be inserted.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-K. Development and characterization of HELIX. a-c, Schematics of type I and type V-K CASTs and HELIX (panels a-c, respectively) and their transposition mechanisms that result in simple insertion or cointegrate gene products. d, Workflow for transposition experiments targeting plasmid substrates. e, Transposition assessed via junction PCRs across the LE/RE at TS1 in pTarget. Experiments were performed with nAniI fused to the N- or C-terminus of TnsB when using pDonor without I-AniI sites. f, Quantification of DNA integration efficiency on plasmids when using ShHELIX and a donor plasmid with a range of distances (d) between the I-AniI site and LE/RE, assessed via ddPCR using miniprepped DNA. g, Coverage of expected insertion products into pTarget from long-read sequencing using a subset of exemplary simple insertion reads for ShHELIX and cointegrate reads for ShCAST (coverage from ShHELIX cointegrate reads and ShCAST simple insertion reads omitted for simplicity). h, Read length distribution when using ShCAST and ShHELIX with a sgRNA targeting TS1 on pTarget from long-read sequencing data. The top right panel is a zoomed-in representation of the ˜8,000 bp read-length peak. i, Comparison of simple insertion and cointegrate product proportions of transposed products forShCAST and ShHELIX constructs when using a pDonor with I-AniI sites 14 bp from the LE/RE and oriented to confer a 5′ nick, assessed via long-read sequencing. j,k, Transposition product purity (panel j) and CFUs (panel k) when using a Lib4 I-AniI site on pDonor (with a distance of 14 bp between the Lib4 sites and the LE/RE), which was previously shown to increase affinity of wild type I-AniI by 5-fold. For panels f and k, mean, SD, and individual data points shown for n=3. TSD, target-site duplication; LE and RE, left and right transposon ends, respectively; sgRNA, single guide RNA; ddPCR, droplet digital PCR.

FIGS. 2A-H. Characterization of DNA insertions on genomic targets using HELIX. a, Workflow for transposition experiments targeting the genome. b, Integration efficiencies when using two different amino acid linkers between nAniI and TnsB, an sgRNA against genomic target site 2 (TS2), and a set of eight donor plasmids with varying distances between the I-AniI sites and the LE/RE, as determined via ddPCR. c, Insertion orientation percentages when using ShCAST or ShHELIX targeting TS2 and using a pDonor with 14 bp spacing between the I-AniI site and the LE/RE d, Integration efficiencies across six genomic target sites for ShCAST and ShHELIX (left panel) and relative integration with ShHELIX normalized to ShCAST (right panel), assessed via ddPCR. e, Coverage of expected insertion products into the genome (TS2) from long-read sequencing using a subset of exemplary simple insertion reads for ShHELIX and cointegrate reads for ShCAST (coverage from ShHELIX cointegrate reads and ShCAST simple insertion reads omitted for simplicity). Transposed products were enriched prior to sequencing via Cas9 targeted enrichment. f, Read-length distribution of transposition products when using ShCAST and ShHELIX on genomic target site 2 (TS2) from long-read sequencing data. The top right panel is a zoomed in representation of the ˜8,200 bp read-length peak. g, Comparison of simple insertion and cointegrate product proportions at TS2 for ShCAST and ShHELIX, assessed via long-read sequencing. h, Integration efficiencies with ShHELIX and the sgRNA targeted to TS5, when using pDonors encoding cargoes of various sizes. Integration assessed via ddPCR. For panels b, d, and h, mean, SD, and individual data points shown for n=3. LE and RE, left and right transposon ends, respectively; sgRNA, single guide RNA; ddPCR, droplet digital PCR.

FIGS. 3A-Q. Extension of HELIX to type V-K CAST orthologs. a, Phylogenetic tree illustrating diversity of TnsB sequences from recently identified Type V-K CASTs²¹, CASTs used in the present study, as well as Tn5053, are noted. b, sgRNA designs for AcCAST. c, Integration efficiencies with AcCAST using two sgRNA designs (from panel b) and a donor plasmid with either native flanking sequence (as previously reported¹⁴) or ShCAST flanking sequence, assessed via ddPCR. d, Schematic of AcHELIX with 14 bp ShCAST flank sequence on pDonor. e, Coverage of insertion products into the genome (TS2) from long-read sequencing, displaying a selection of exemplary simple insertion reads for AcHELIX and cointegrate reads for AcCAST (coverage from AcHELIX cointegrate reads and AcCAST simple insertion reads omitted for simplicity). Transposed products were enriched prior to sequencing via Cas9 targeted enrichment. f, Read-length distribution of transposition products when using AcCAST and AcHELIX on TS2 from long-read sequencing data. The top right panel is a zoomed in representation of the ˜8.3 kb read-length peak. g, Comparison of simple insertion and cointegrate product proportions for AcCAST and AcHELIX, assessed via long-read sequencing. h,i, Integration efficiencies in the T-LR and T-RL orientations (panels h and i, respectively) across six genomic target sites for AcCAST and AcHELIX, assessed via ddPCR. In panel h, AcHELIX T-LR integration efficiency relative to AcCAST is shown in the right panel. All transformations contain the pDonor variant with ShCAST flanks and 14 bp spacing between the nAniI sites and LE/RE. j, Integration efficiencies when using AcHELIX using the sgRNA targeted to TS6 and pDonors encoding cargoes of various sizes, assessed via ddPCR. k, Schematic of ShoHELIX with 14 bp ShCAST flank sequence on pDonor. 1, Coverage of expected insertion products into the genome (TS2) from long-read sequencing, displaying a selection of exemplary simple insertion reads for ShoHELIX and cointegrate reads for ShoCAST (coverage from ShoHELIX cointegrate reads and ShoCAST simple insertion reads omitted for simplicity). Transposed products were enriched prior to sequencing via Cas9 target enrichment. m, Read-length distribution when using ShoCAST and ShoHELIX on a genomic target (TS2) from long-read sequencing data. n, Comparison of simple insertion and cointegrate product proportions for ShoCAST and ShoHELIX, assessed via long-read sequencing. o,p, Integration efficiencies in the T-LR and T-RL orientations (panels o and p, respectively) across six genomic target sites for ShoCAST and ShoHELIX, assessed via ddPCR. q, Integration efficiencies when using ShoHELIX with a TS3-targeted sgRNA and pDonors encoding cargoes of various sizes, assessed via ddPCR. All ShoCAST and ShoHELIX transformations contain a pDonor variant with ShCAST flanks. For panels c, h-j, and o-q, mean, SD, and individual data points shown for n=3. LE and RE, left and right transposon ends, respectively; sgRNA, single guide RNA.

FIGS. 4A-L. Specificity profiling of ShCAST and ShHELIX systems. a, Schematic of 2- and 3-component ShCAST systems containing Cas12k fusions. b, Relative integration efficiencies with 3- and 2-component ShCAST systems using TnsC and/or TniQ fusions to Cas12k. c, Schematic of 3-component ShHELIX systems containing Cas12k fusions. d, Relative integration efficiencies for 3-component ShHELIX systems. e, Integration efficiencies of ShCAST and ShHELIX systems with or without Cas12k-TnsC fusion when using a target plasmid with a pre-inserted transposon. f, On-target specificity of ShCAST and ShHELIX systems in Endura cells (pir⁻) and PIR2 cells (pir⁺) with the genome-targeting TS2 sgRNA, measured by an unbiased specificity profiling approach (see Methods). g, Schematic of transformation protocol when using pi protein coexpression in Endura (pir⁻) cells. h, On-target specificity of ShCAST and ShHELIX with or without pi protein coexpression with the genome-targeting TS2 sgRNA i-l, Visualization of genome-wide integration events in Endura cells when using ShCAST (6.67M reads; panel i), ShHELIX with a Cas12k-TniQ fusion (4.44M reads; panel j), ShHELIX with a Cas12k-TnsC fusion (3.29M reads; panel k), or ShHELIX with pi protein coexpression (7.31M reads; panel 1) when programmed with the TS2 sgRNA. Filled triangles under the x-axis indicate the on-target site; y-axis represents the percentage of reads mapping to any given genomic site. For panels b, d, and e, mean, SD, and individual data points shown for n=3. LE and RE, left and right transposon ends, respectively; PAM, protospacer-adjacent motif.

FIGS. 5A-L. HELIX-mediated DNA insertion in human cell lysates and human cells. a, Schematic of N₇HELIX with 14 bp ShCAST flank sequence on pDonor. b, Workflow of plasmid targeting transposition experiments in human cell lysates. c, qualitative assessment of integration via junction PCR across LE and RE using purified pTarget from lysate assays. d, Representative Sanger sequencing reaction of a PCR reaction of an insertion product (from panel c). e, PAM-to-LE insertion distance profile of N₇HELIX with TS1 sgRNA from plasmid-targeting experiments in a HEK 293T lysate (assessed by NGS; see FIG. 12A). f, Comparison of simple insertion and cointegrate product proportion for N₇CAST and N₇HELIX, assessed via PCR enrichment of total and cointegrate insertions and subsequent long-read sequencing (Example 11). g, Schematic of workflow for plasmid-targeting experiments in HEK 293T cells, using five separate plasmids. The N₇CAST or N₇HELIX proteins were all expressed from a single all-in-one plasmid. Two different sgRNA architectures (the sgRNA1 scaffold sequence is wild-type, while the sgRNA2 scaffold contains substitutions within poly-T stretches relative to sgRNA1 to enable U6 promoter compatibility) using different promoters were tested, both targeting TS1. h, Junction PCR and Sanger sequencing across LE using insertion products from HEK 293T cell-based plasmid-targeting assays. i, Quantification of integration efficiency when transfecting various amounts of pTarget, from HEK 293T cell-based plasmid-targeting assays and assessed via ddPCR. j, Quantification of integration efficiency when coexpressing HU protein (in addition to S15), from HEK293T cell-based plasmid-targeting assays and assessed via ddPCR. k, Integration efficiency of N₇CAST and N₇HELIX when targeting endogenous genomic target sites in HEK 293T cells, assessed via ddPCR. l, Schematic of areas of potential optimization to increase the integration efficiency of CASTs and HELIX systems in human cells. For panels i-k, mean, SD, and individual data points shown for n=3. LE and RE, left and right transposon ends, respectively; PAM, protospacer-adjacent motif, sgRNA, single guide RNA; NT, non-targeting; HH, Hammerhead Ribozyme; HDV, Hepatitis delta virus ribozyme.

FIGS. 6A-D. Characterization of TnsA fusions to ShTnsB. a, Structures of various TnsA enzymes, either experimentally solved (E. co/i TnsA; PDB 1F1Z) or computationally predicted via AlphaFold. b, Integration efficiencies when targeting genomic site TS2 using either ShCAST (no fusion) or variants containing fusions of TnsA and ShTnsB linked by either a short GSG or XTEN linker. Integration measured by ddPCR; mean, SD, and individual data points shown for n=3. c, On-target cointegrate characterization as measured by long-read sequencing, following a Cas9-based target enrichment protocol. d, Proportion of total insertions that occur in the pEffector plasmid when using either no fusion (ShCAST), nAniI fusion (ShHELIX), or TnsA fusions.

FIGS. 7A-D. Optimization and characterization of plasmid-targeting experiments. a, Schematic of donors bearing modified flank sequences with I-AniI sites positioned at various distances from the left and right transposon ends (LE/RE, respectively). b, Colony-forming units (CFUs) from transformations with ShCAST and ShHELIX plasmids targeting TS1 when using a series of pDonor plasmids bearing various spacings between the I-AniI sites and LE/RE. c, Integration efficiencies when using ShCAST targeting TS1 and a series of pDonors with different LE/RE flank sequences (corresponding to the ShHELIX pDonors bearing different spacings between the I-AniI sites and the LE/RE; see panel a), assessed via ddPCR. d, Alignment of ten exemplary reads bearing ShHELIX-mediated cargo integration 62 bp downstream of the PAM on pTarget. For panels b and c, mean, SD, and individual data points shown for n=3. LE and RE, left and right transposon ends, respectively.

FIG. 8. Workflow for plasmid enrichment prior to long-read sequencing. Schematic of the protocol to enrich for transposed plasmid products to improve read-depth of intended products via long-read sequencing. sgRNA, single guide RNA; LE and RE, left and right transposon ends, respectively.

FIGS. 9A-D. Characterization of Y2 ShHELIX. a, Colony-forming units (CFUs) from transformations with Y2 ShHELIX plasmids targeting TS1 when using a series of pDonor plasmids bearing various spacings between the I-AniI sites and LE/RE. Mean, SD, and individual data points shown for n=3. b, Coverage of expected insertion products into pTarget from long-read sequencing, displaying an exemplary subset simple insertion or cointegrate reads for Y2 ShHELIX. c, Read length distribution when using ShCAST and Y2 ShHELIX with a sgRNA targeting TS1 on pTarget. d, Comparison of simple insertion and co-integrate product proportions via long-read sequencing for various conditions using Y2-ShHELIX targeting TS1. LE and RE, left and right transposon ends, respectively.

FIGS. 10A-C. ShHELIX control experiments. a, Comparison of simple insertion and co-integrate product proportions via long-read sequencing for a HELIX variant with a catalytically attenuated nAniI (dShHELIX) and when using HELIX with a pDonor without I-AniI sites. b, Comparison of simple insertion and co-integrate product proportions via long-read sequencing for ShCAST and ShHELIX when using a pDonor with flipped I-AniI sites that place the nAniI nicking sites on the same strand as the nick from TnsB. c, Potential alternative mechanism enabling simple insertion products when using a pDonor containing a flipped I-AniI site. TSD, target site duplication.

FIGS. 11A-B. Integration efficiency based on long-read sequencing. a, Comparison of integration efficiencies for each system as measured by ddPCR or by Cas9-enriched long-read sequencing. The dashed grey line denotes the diagonal (agreement between the two types of measurements). b, Integration efficiencies at TS2 when using CAST and HELIX systems, assessed via long-read sequencing. Stacked bars represent the fraction of Cas9-enriched target reads that lack or contain the cargo insertion. Integration (colored portion of each bar) represents the number of reads that contain the cargo insertion divided by the total number of targeted reads.

FIGS. 12A-M. Cargo insertion distance from the PAM. a, Schematic of the workflow to characterize PAM-to-LE insertion distances via next-generation targeted sequencing. PAM-to-LE insertion distance profiles for various CAST and HELIX constructs shown in panels: b, ShCAST (4-components); c, ShHELIX (4-components); d, AcCAST (4-components); e, AcHELIX (4-components); f, ShoCAST (4-components); g, ShoHELIX (4-components). h, ShCAST with Cas12k-TniQ (3-components); i, ShCAST with Cas12k-TniQ-TniQ (3-components); j, ShCAST with Cas12k-TnsC (3-components); k, ShHELIX with Cas12k-TniQ (3-components); 1, ShHELIX with Cas12k-TniQ-TniQ (3-components); m, ShHELIX with Cas12k-TnsC (3-components); sgRNA, single guide RNA; PAM, protospacer adjacent motif, LE and RE, left and right transposon ends, respectively; NGS, next-generation sequencing.

FIGS. 13A-C. Comparison of type I INTEGRATE and type V-K CAST and HELIX systems. a, Schematic of conditions and constructs tested, controlling for growth time (24 hrs), donor cargo size (2.1 kb), approximate donor copy number (high copy), bacterial strain (PIR1), general target location (closest compatible PAMs near genomic target sites TS2, TS5, and TS6), and efficiency measurement method (ddPCR). b,c, Integration efficiencies of INTEGRATE, CAST, and HELIX in the intended forward orientation (panel b) or in the unintended reverse orientation (panel c). For panels b and c, mean, SD, and individual data points shown for n=3.

FIGS. 14A-B. Integration efficiencies for more minimal CAST and HELIX systems. a, b, Absolute integration efficiencies when targeting the genome at TS2 for 2-, 3-, or 4-component ShCASTs (panel a), and when targeting TS2 or TS5 for 3- and 4-component ShHELIX systems (panel b). For both panels, integration efficiencies were assessed via ddPCR and used to calculate relative integration as shown in FIG. 3; mean, SD, and individual data points shown for n=3.

FIGS. 15A-D. Genome-wide integration profiles of ShCAST and ShHELIX systems. a-d, Integration site profiles from unbiased genome-wide insertion analysis of various CAST and HELIX constructs. The experiments were performed in Endura cells (panels a and b) or PIR2 cells (panels c and d), using various ShCAST configurations (panels a and c) or ShHELIX configurations (panels b and d) including different donor architectures, fusions to Cas12k, pi coexpression, or I-AniI variants.

FIG. 16. Influence of pDonor copy number and pi protein type on integration efficiency. Integration efficiencies using ShCAST and ShHELIX and an sgRNA targeting genomic site TS2 in two different bacterial strains that express either wild-type pi protein (pir) or a mutant copy-number mutant (pir116) (where PIR1 and PIR2 cells maintain pDonor at approximately 250 and 15 copies, respectively). Integration efficiencies assessed via ddPCR; mean, SD, and individual data points shown for n=3. R6Kg, origin of replication that requires the gene, pir, to replicate.

FIG. 17. Coding sequence and component number comparison of CAST and HELIX systems. Approximate sizes of coding sequences and number of protein subunits for prototypical type I and type V-K CASTs, HELIX systems developed in this study, as well as a recently described mini CAST from metagenomic mining⁹. nAniI, nicking I-AniI (K227M).

FIGS. 18A-E. Additional characterization of N₇CAST and N₇HELIX. a, Schematic of the genomic architecture of N₇CAST as found in Nostoc Sp. PCC7107 (identified by Strecker et al.⁷; not drawn to scale). b, PAM-to-LE insertion distance profile when using N₇CAST and an IVT sgRNA targeting TS1 on pTarget in lysate experiments, assessed by NGS. c, Schematic of all-in-one N₇CAST and N₇HELIX expression plasmids, and two versions of the sgRNA that either encode the canonical N₇ scaffold expressed from a U6 promoter (sgRNA1), or a derivative where poly-T stretches in the scaffold are substituted to be more compatible with transcription from the U6 promoter (sgRNA2). d, Junction PCRs when using N₇CAST or N₇HELIX with either IVT sgRNA1 or sgRNA2 targeting TS1 on pTarget in HEK 293T lysate experiments. e, Junction PCRs from HEK 293T cell-based plasmid-targeting experiments with or without N₇ or E. coli (Ec) S15 and pi proteins.

FIG. 19. Exemplary pDonor sequences. I-AniI sites are shown in bold font. The LE and RE sequences for ShCAST, AcCAST, ShoCAST, and N7CAST are condensed for brevity in the pDonor sequences, but their sequences also shown in the table.

### DETAILED DESCRIPTION

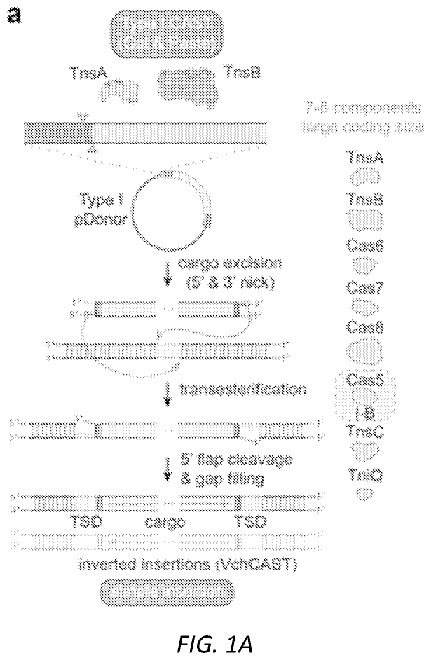

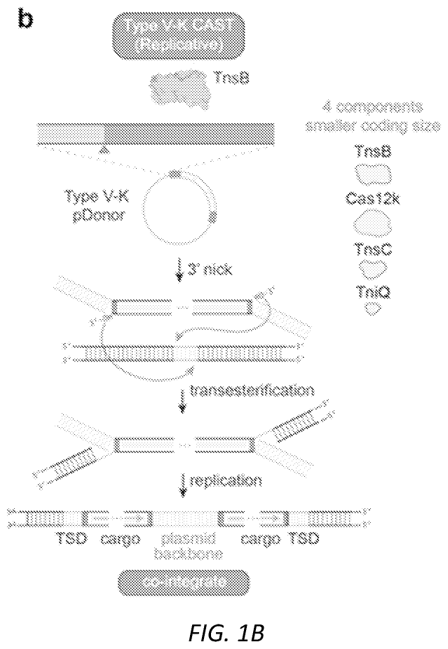

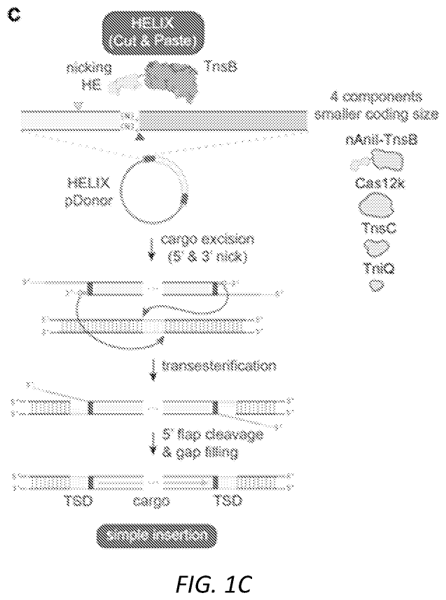

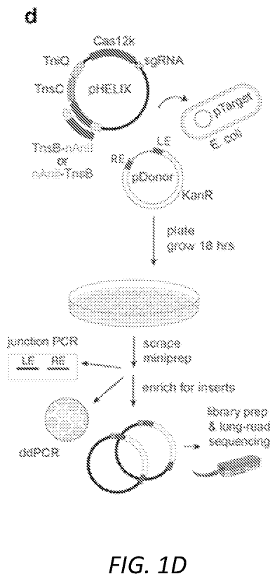

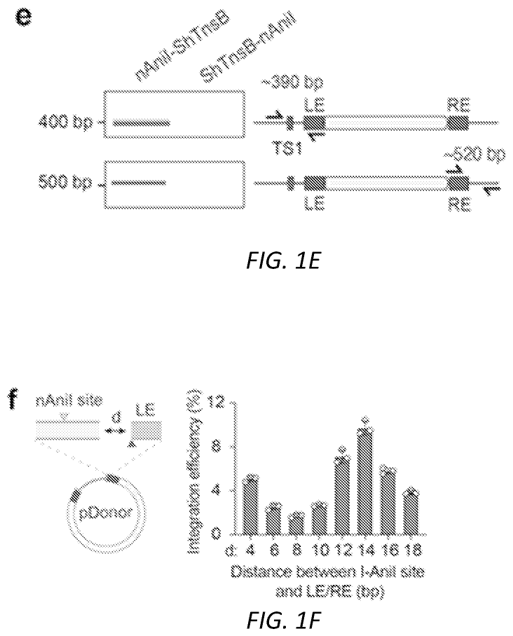

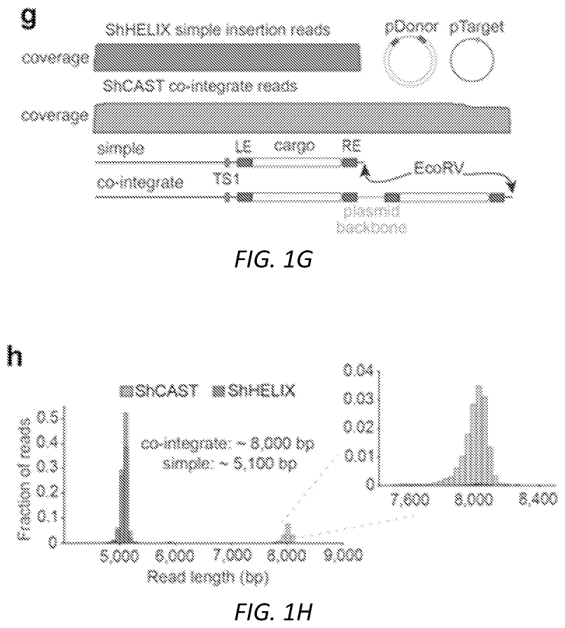

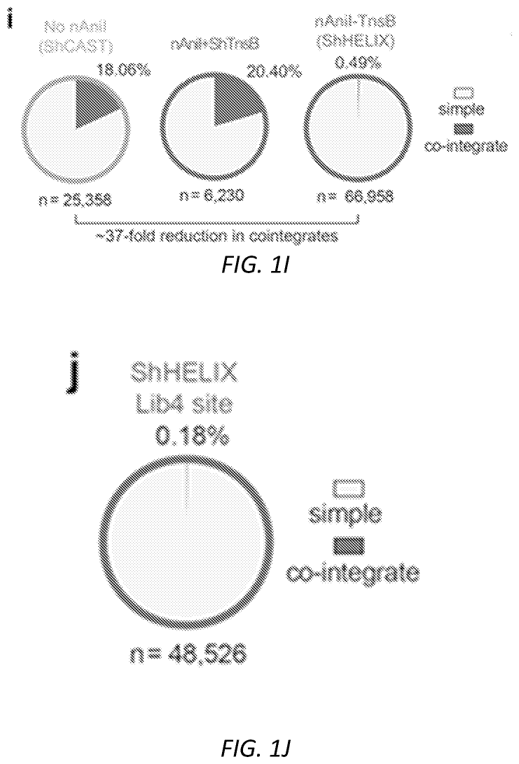

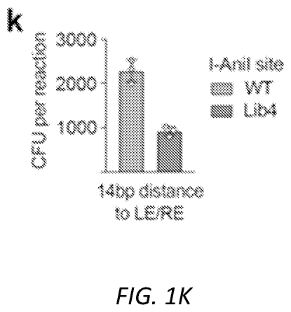

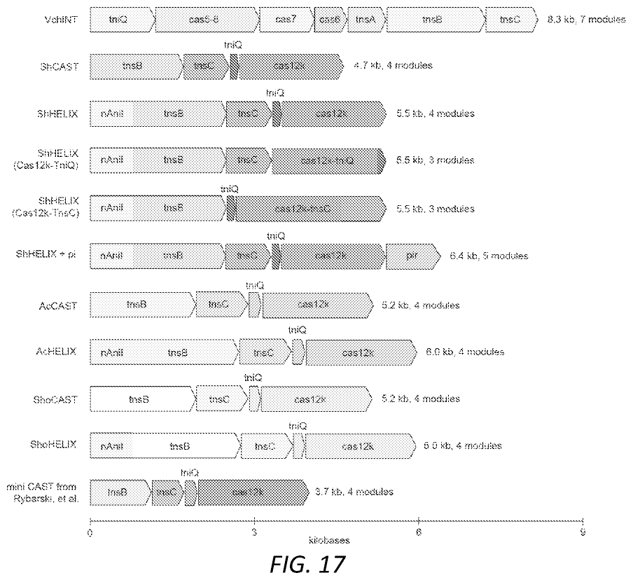

CRISPR-associated transposases (CASTs) are an emergent class of genome editing technologies that enable programmable DNA insertions without reliance on recombination, sequence-specific recombinases, or DSBs. However, the currently discovered and characterized systems have limitations that restrict their ease of use, including size (FIG. 17), stoichiometric and component complexity, and/or insertion product purity. The two main classes of CASTs, types I and V-K, have distinct and complementary properties. While characterized type I CASTs exhibit high on-target specificity and generally only result in the intended simple insertion gene products¹⁷ (though with exceptions¹⁸), the larger number of Cas genes, stoichiometric complexity, and large coding size may limit downstream tool development in other organisms such as eukaryotic cells. Additionally, the tendency of some type I systems to result in bidirectional insertions leads to undesirable edit impurity¹⁵ (FIG. 1a). In comparison, type V-K CASTs are more compact in terms of coding size, contain only four core components, and result in complete or near-complete unidirectional insertions^{14,16}. However, type V-K CASTs lead to a problematic mixture of simple insertion and cointegrate gene products, the latter of which consists of cargo duplication and full plasmid backbone insertion^{4,6,19} (impacting desired product ‘purity’) (FIG. 1b). Additionally, compared to type I systems, type V-K CASTs exhibit substantially lower integration specificity^{14,16,17,20}.

Another major difference between type I and type V-K CASTs is whether they encode or lack TnsA, respectively (though type I systems can also lack TnsA in rare cases²¹), a distinction that contributes to their disparate integration product purities (defined as the ratio between simple insertions and cointegrate products). In both Tn7 transposons and type I CASTs, TnsA and TnsB carry out 5′ and 3′ donor nicking, respectively, resulting in simple insertions via cut-and-paste transposition (FIG. 1a). In Tn5053 transposons and type V-K CASTs, which lack TnsA, and also in Tn7 transposons and modified type I systems with catalytically dead TnsA^{17,22}, only 3′ donor nicking occurs via TnsB. Singly-nicked donors result in a substantial fraction of cointegrate insertions through replicative, instead of cut-and-paste, transposition²³ (FIG. 1b). To overcome the lack of TnsA in type V-K systems, we hypothesized that orthogonal DNA nickases could be leveraged to restore 5′ donor nicking. An ideal nickase would be small (to add minimal coding size to the system), have predictable nicking sites and strand preference, and would function in various organisms for downstream tool development and applications. Potential nickases to consider include orthogonal TnsA enzymes from type I CASTs or other transposons^{17,24}, nicking restriction endonucleases²⁵, nicking Cas variants^{9,26,27}, phage HNH endonucleases²⁸, or nicking homing endonucleases (nHEs)²⁹⁻³².

For genome editing applications, an ideal DNA insertion technology would generate programmable, high specificity, unidirectional, recombination-independent, and pure simple insertion products, all with few components and a minimal coding sequence. Therefore, we sought to develop an engineered CAST that combines the simplicity and orientation predictability of type V-K systems with the product purity and specificity of type I systems. Our results reveal that an optimized and engineered HE-assisted Large-sequence Integrating CAST-compleX (HELIX), comprised of a nHE fusion to TnsB along with the remaining CAST components, can substantially improve the purity and specificity of CAST-mediated DNA insertions.

As shown herein, HELIX harnesses the technological advantages of type V-K CASTs and employs a nHE fusion and a modified donor plasmid to achieve programmable and efficient cut-and-paste DNA insertion similar to type I CASTs. HELIX dramatically increased simple insertion product purity on plasmid and genomic targets in E. coli and retains robust RNA-guided transposition at or near wild-type levels. Additionally shown herein is simplified CAST and HELIX systems comprising 3-component systems via subunit fusions to Cas12k, which will increase integration efficiencies.

CASTs are an emergent class of genome editing technologies that enable programmable DNA insertions without reliance on recombination, sequence-specific recombinases, or DSBs. Here we overcome some of the major limitations of CASTs by developing HELIX, which harnesses the technological advantages of type V-K CASTs to achieve programmable, specific, and efficient cut-and-paste DNA insertion. We demonstrate that HELIX increases simple insertion product purity on plasmid and genomic targets in E. coli and retains robust RNA-guided transposition at or near wild-type levels. HELIX is efficacious across several type V-K CAST orthologs, establishing the universality of this approach. We also demonstrate that HELIX is substantially more specific than its derived CAST, and that Cas12k fusions and/or pi protein coexpression can further reduce genome-wide off-target integration. Finally, we demonstrate that the advantages of HELIX can translate into human cell contexts on plasmid targets. Together, our approaches are the first descriptions of CAST engineering and highlight how other naturally occurring enzymes can be leveraged to augment CAST properties for uses in various systems.

Our results also provide insight into certain mechanistic aspects of HELIX. First, nAniI must be proximal to TnsB via fusion to reduce cointegrates, potentially to coordinate nAniI and TnsB nicking reactions. Similarly, in Tn7 and type I CASTs, physical proximity is mediated by protein-protein interactions between TnsA, TnsB, and TnsC³³. Secondly, fusions of TnsA domains from Tn7 or type I CASTs to ShTnsB were ineffective at reducing cointegrates, likely because TnsA is only active in complex with its cognate TnsB and TnsC to physically and temporally coordinate strand specific cleavage^{24,33}. These results suggest that generating the 5′ nick in type V-K systems via fusion proteins to TnsB is optimal from standalone nicking endonucleases (such as an nHE in HELIX); a conclusion supported by our efficiency and target immunity datasets which reveal that nAniI-TnsB fusions do not substantially interfere with other CAST components (i.e. donor or target DNA, or TnsC).

The continued discovery and optimization of CASTs will lead to more robust integration technologies. We envision identification of new systems with useful characteristics (e.g. via metagenomic mining for more compact type V-K systems²¹) will contribute to the diversity of enzymes that can be further engineered via HELIX or other methods to enhance various integration parameters. Amidst our characterizations, we discovered various areas of optimization to modulate CAST properties. For instance, modification of the flanking sequencing directly adjacent to the LE/RE on pDonor can influence integration, perhaps due to sequence-specific effects (as has been demonstrated for mu transposase⁵²) and/or altered interactions with unknown host factors. Furthermore, fusion proteins to various CAST components led to unexpected alterations in properties. Our findings suggest that a better understanding of several parameters (augmenting the donor flanking sequences, amino acid linkers, spacings between nHE sites and LE/RE, nHE selection, etc.) combined with efforts to create hyperactive variants of type V-K CASTs (potentially through TnsB and Cas12k directed evolution and structure-guided engineering) will lead to more potent next-generation CAST and HELIX systems.

While HELIX solves many limitations of V-K CASTs, our work also leaves open questions that merit continued investigation. The incomplete ablation of cointegrate products may result from uncoordinated donor nicking by nAniI and TnsB, which may also be the case for observed, though minimal, cointegrate products in type I systems potentially due to asynchronous TnsA and TnsB donor nicking¹⁷. Additional studies to investigate the mechanisms of the various HELIX improvements would be worthwhile, including how pi protein or fusions (nAniI-TnsB, Cas12k-TnsC, Cas12k-TniQ, etc.) contribute to specificity modulation. We hypothesize that alterations in CAST conformation via nAniI-TnsB fusion and altered donor topology via modified TnsB-donor interaction and pi binding of iteron and/or AT-rich sequences⁵³ in the left and right transposon ends and/or parts of the donor backbone are crucial factors. Moreover, how component fusions and/or pi protein work in concert with HELIX, but generally not CAST, to increase specificity warrants further study.

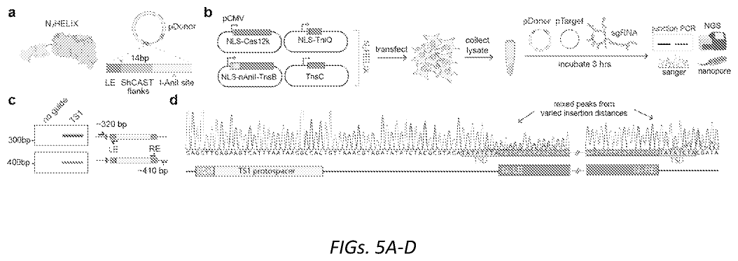

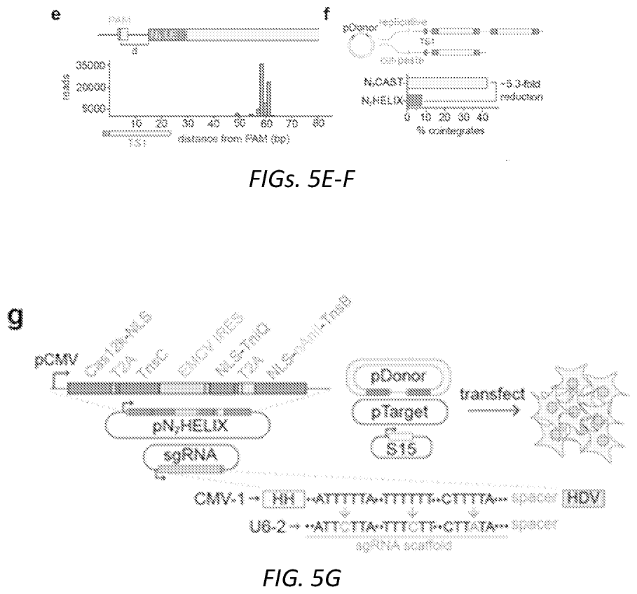

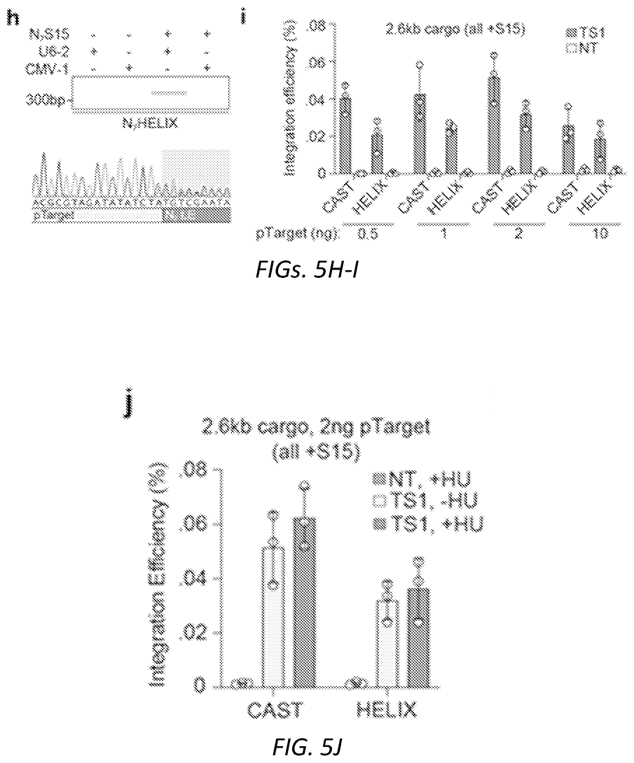

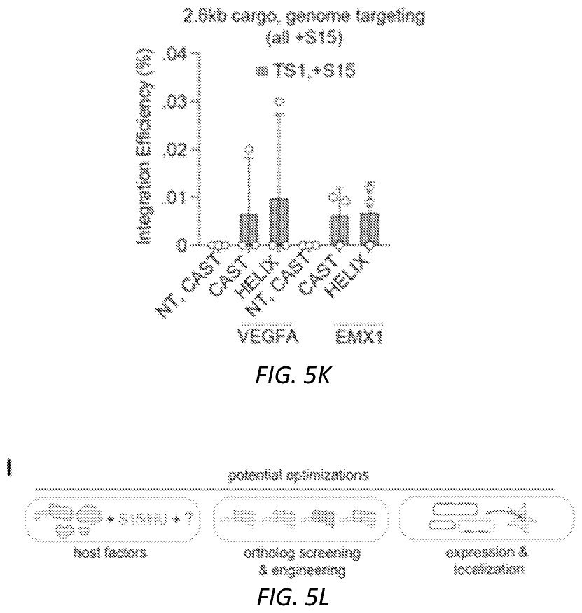

Although we demonstrate that CASTs and HELIX can function in human lysate and cells on plasmid targets, integration efficiency was low using described constructs and conditions. Methods that can improve efficiency are therefore critical for translation of these systems in various contexts. The recent discovery that ribosomal protein S15 is a bacterial host factor required for efficient transposition⁴³ makes it plausible that additional bacterial host protein(s) may be necessary for efficient human cell integration. Our results corroborate the necessity of S15. Indeed, the nucleoid-associated proteins (NAPs) HU and IHF are required for efficient Mu transposition⁵¹, and the same and/or other NAPs and DNA-bending proteins are a transposition requisite or enhancement for other transposon families (e.g. Tn10, IS903, Tn552, Sleeping Beauty, etc)⁵⁴⁻⁵⁶. Pi protein, which we observed to enhance insertion specificity, is also known to distort DNA⁵³, and can act as a competitive binder with IHF⁵⁷. Thus, protein-induced changes in donor topology can affect transposition characteristics—perhaps in addition to specificity, paired complex formation and/or transposase activity. Furthermore, host-encoded acyl-carrier protein (ACP) and ribosomal protein L29, have been shown to participate in TnsD-mediated Tn7 transposition⁵⁸ and DnaN in the TnsE-mediated pathway⁵⁹. Along with host factor discovery, engineering and optimization of the HELIX components via modifications to the donor, the sgRNA, and the proteins themselves (e.g. more active nHEs³⁵ and TnsB variants, Cas12k variants with improved binding affinity, etc.) should enable more efficient and specific human genome targeting (FIG. 5j), as has been done with other Cas orthologs including some that initially displayed minimal activity⁶⁰⁻⁶². Component fusions may also prove useful in facilitating localization of these multi-component systems.

Beyond CASTs, other advances have occurred in DSB-free large sequence integration technologies. Recent studies combined prime editing (PE) with site-specific serine recombinases to integrate DNA into the human genome in a RNA-programmed manner^{63,64}. Upon successful discovery and engineering efforts to enable more efficient use in human cells, HELIX represents a complementary technology with advantages compared to PE-based methods: a smaller coding size, a need to design only a single sgRNA instead of multiple pegRNAs, a complete elimination of DSBs, a more minimal dependence on host cell repair, and a vast diversity of CASTs that may be naturally suited for efficient eukaryotic function and therapeutic deliverability.

### Transposon-Nickase Fusion Proteins

Described herein are fusion proteins comprising a transposition protein B (TnsB) protein (e.g., Tn7, Tn7-like, or Tn5053-like transposition protein B (TnsB) protein) fused to a protein (such as, a nickase), optionally via an intervening linker. In some embodiments, a DNA cleavase fusion can be used instead of a nickase fusion for cut-and-paste DNA insertion. The present methods and compositions can be applied in a number of transposon/CAST systems, e.g., in the following.

### Canonical Tn7 Transposon^{42,43,44}

Tn7 has four components TnsABCD. TnsABC forms a heterotrimeric complex (TnsA and TnsB create 5′ and 3′ nicks at the transposon ends and TnsC is an ATPase that regulates transposition activity). Tn7 is targeted to DNA via two alternative pathways: (1) mediated by TnsD, a sequence-specific DNA binding protein which recognizes the Tn7 attachment site^{45,46} (2) mediated by TnsE, which facilitates transposition into conjugal plasmids and replicating DNA⁴⁷.

CRISPR-Cas Systems Associated with Tn7-Like Transposons (Type I CASTs):

Type I CRISPR Cas systems are associated with Tn7-like transposons, containing TnsA, TnsB, TnsC, and TniQ genes and the CRISPR system. TnsD/TnsE in canonical Tn7 transposons is replaced by these CRISPR-Cas systems. “Tn7-like” denotes relatedness to the canonical system (i.e., to the Tn7 family of transposons) and includes components TnsABC. Such systems can include VchCAST (from Vibrio cholerae Tn6677), AsaCAST (from Aeromonas salmonicida S44), AvCAST (from Anabaena variabilis ATCC 29413), PmcCAST (from Peltigera membranacea cyanobiont 210A) and PtrCAST in BL21(DE3).⁵⁷

CRISPR-Cas Systems Associated with Tn5053 Family of Transposons (Type V-K CASTs):

Type V-K CASTs are most closely related to the Tn5053 family of transposons^{48,21}. Such systems can include shCAST (from Scytoneia hofmannii), AcCAST (from Anabaena cylindrica), ShoCAST (from Scytonema hofmannii PCC 7110). Tn5053 transposons have not been fully characterized, but are known to lack TnsA—which results in cointegrates that are resolved by a transposon-encoded recombinase, TniR⁴⁹. For type V-K CASTs, the transposon does not encode an identifiable resolvase/recombinase to do so. In some embodiments, the Type V-K CAST is a CAST as described in Rybarski J R, Hu K, Hill A M, Wilke C O, Finkelstein I J. Metagenomic discovery of CRISPR-associated transposons. Proc Natl Acad Sci USA. 2021 Dec. 7; 118(49):e2112279118. doi: 10.1073/pnas.2112279118, or in Table 2 of U.S. patent Ser. No. 11/384,344B2.

### Nickases/Cleavases

The nickase can be fused to either the N or C terminus of the transposon. Preferably the nickase is smaller than about 500 amino acids. A number of suitable nickases are known in the art and can be used; exemplary nickases include nicking restriction endonucleases²², nicking Cas variants^{9,23,24}, or phage HNH endonucleases²⁵, or the catalytic portion of TnsA enzyme from type I CASTs or Tn7 transposons²⁶ or a catalytic portion thereof. In some embodiments, the nickase is a homing endonuclease (HE), e.g., a LAGLIDADG HE (LHE); for example, the LHE from Aspergillus nidulans (I-AniI), optionally comprising a K227M mutation (nAniI) or a hyperactive variant thereof (e.g., Y2 I-AniI), can be used. Examples of additional homing endonucleases (categorized based on sequence motifs/domains) include: LAGLIDADGs, e.g., I-SceI (which has been engineered to be a sequence specific nickase⁴⁹) and I-DmoI (also been engineered to be a sequence specific nickase⁵⁰); H—N—H, e.g., I-PfoP3I (which naturally occurs as a nickase)⁵¹ and I-BasI (also naturally occurs as a nickase); GIY-YIG, e.g., I-BmoI⁵ and I-TevI14; or His-Cys Box, e.g., I-PpoI⁵². For a comprehensive review see Stoddard et al., 2011¹⁶. As noted above, in some embodiments, fusions of cleavase versions of these enzymes to a transposon protein, e.g., TnsB, are used, which might improve integration product purity and reduce co-integrants.

### Linkers

In some embodiments, the fusion proteins comprise a linker between the transposon protein and the nickase. Linkers as known in the art can be used, e.g., comprising 1-100 amino acids, e.g., flexible linkers (e.g., XTEN linkers (comprising GEDSTAP (SEQ ID NO: 1) amino acids) or Gly-Ser or Gly-Ser-Ala rich linkers (e.g., GSAGSAAGSGEF (SEQ ID NO:2), GGSGGGSGG (SEQ ID NO:3), (GGGGS)₃ (SEQ ID NO:4) or (Gly)ₙ (SEQ ID NO:5)), PAS repeats, GQAP (SEQ ID NO:6)-like repeats, or SOBI (SEQ ID NO:7) linkers; or rigid linkers, e.g., alpha helical linkers (e.g., (EAAAK)₃) (SEQ ID NO:8)or (XP)ₙ (SEQ ID NO: 9), with X designating any amino acid, preferably Ala, Lys, or Glu. See, e.g., Chen et al., Advanced Drug Delivery Reviews, 15 Oct. 2013, 65(10):1357-1369; An Overview of Linkers for Recombinant Fusion Proteins, kbdna.com/publishinglab/lnkr (05/08/2021); Podust et al., Protein Engineering, Design & Selection (2013), 26 (11), 743-753; Kjeldsen et al., ACS Omega 2020, 5, 31, 19827-1983.

### Flanking Sequences

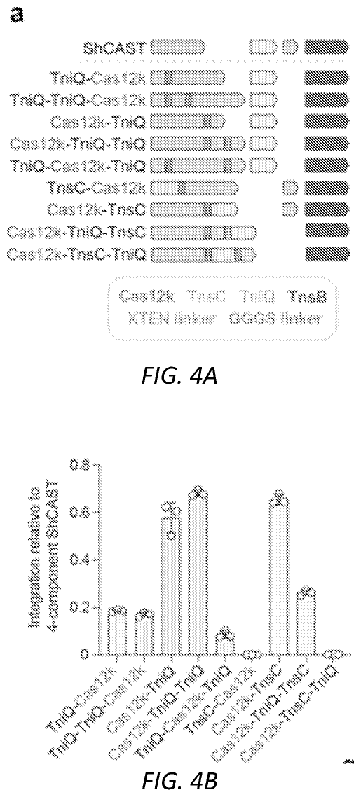

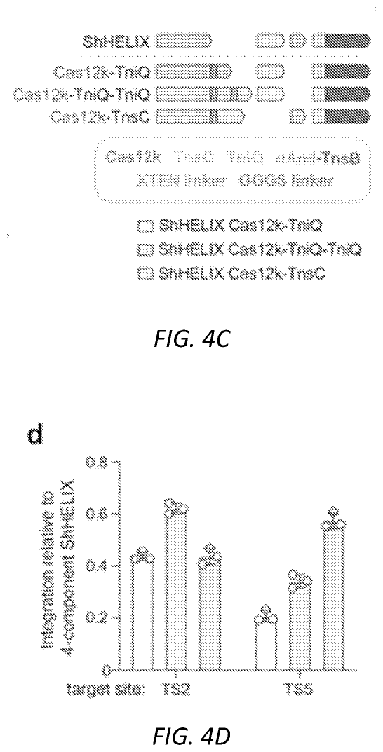

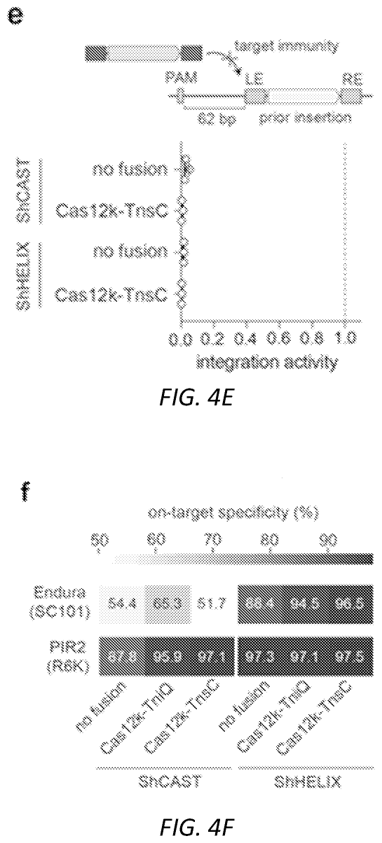

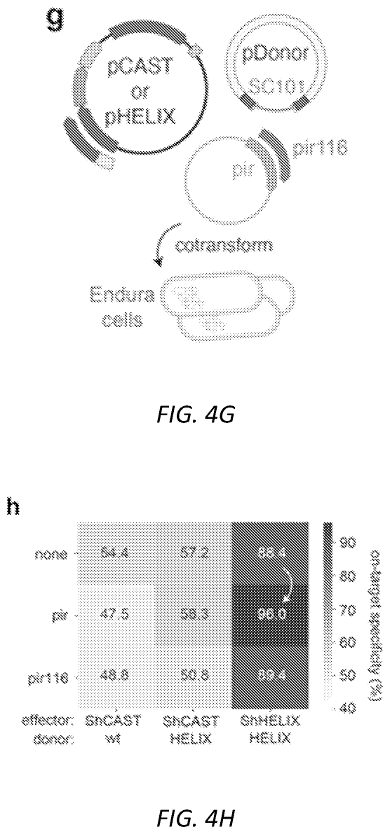

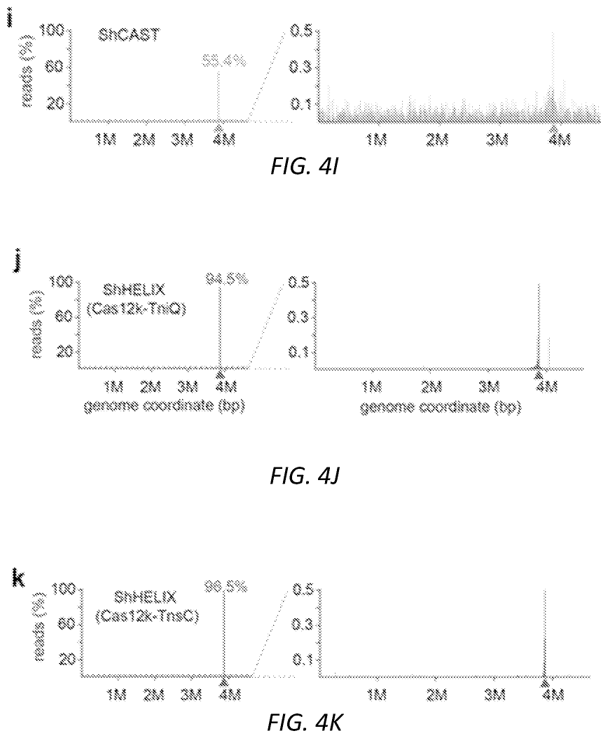

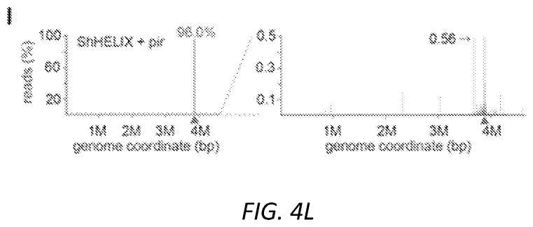

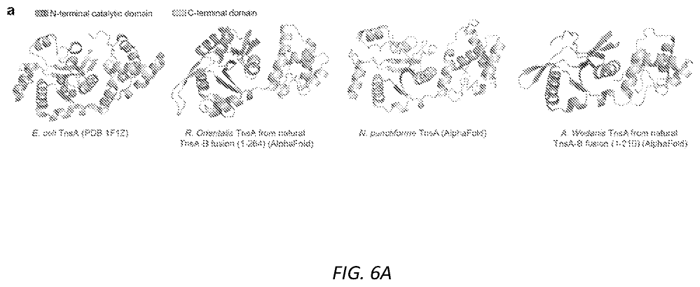

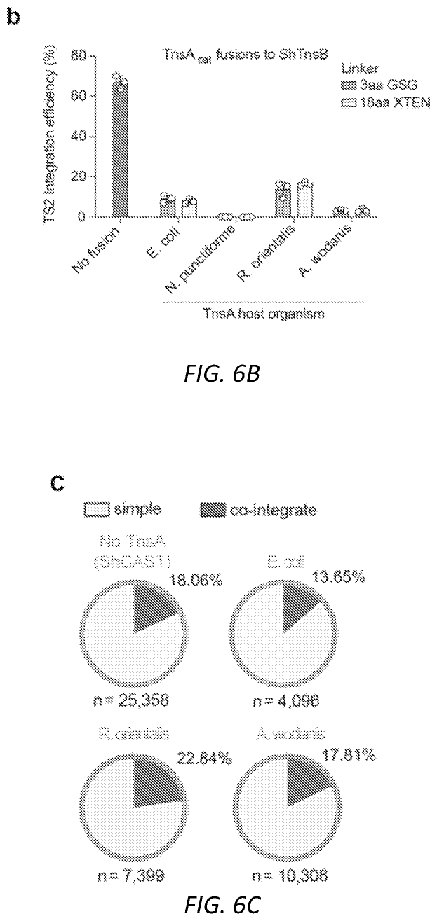

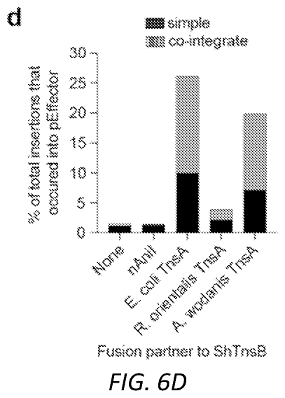

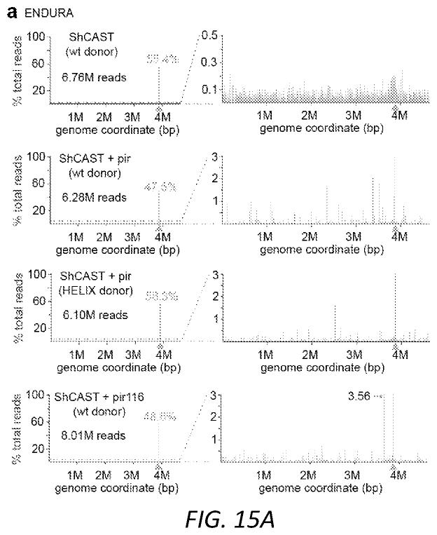

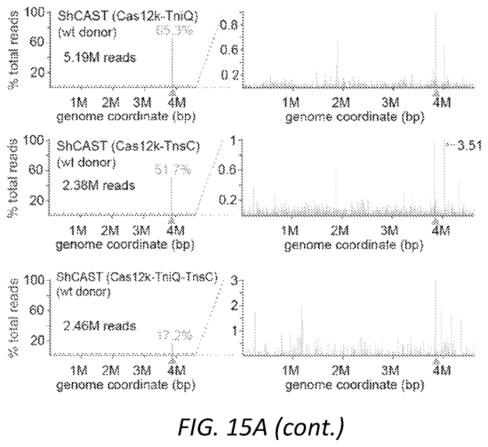

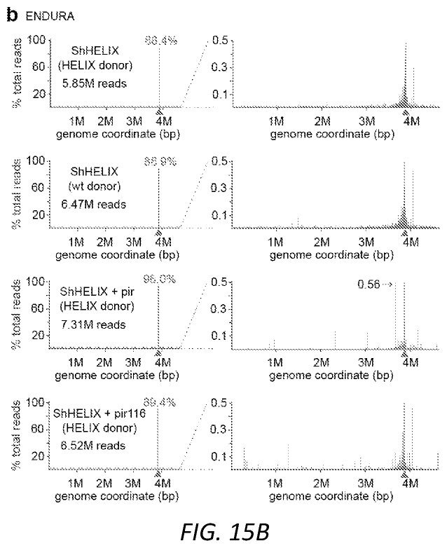

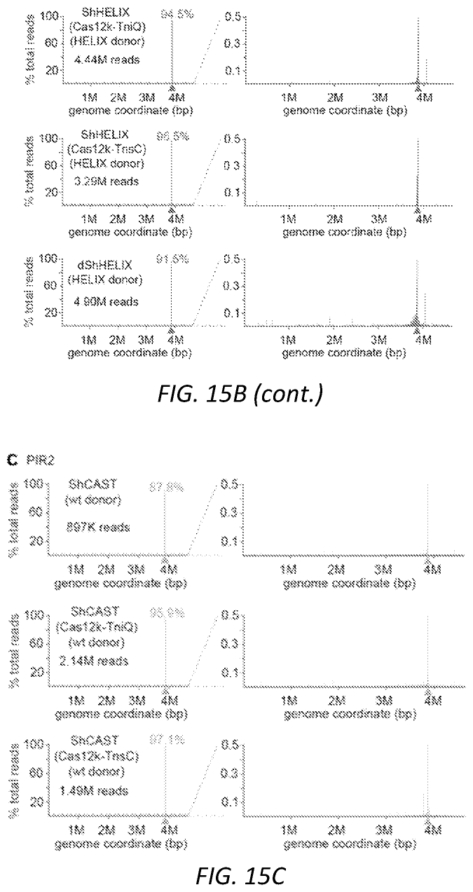

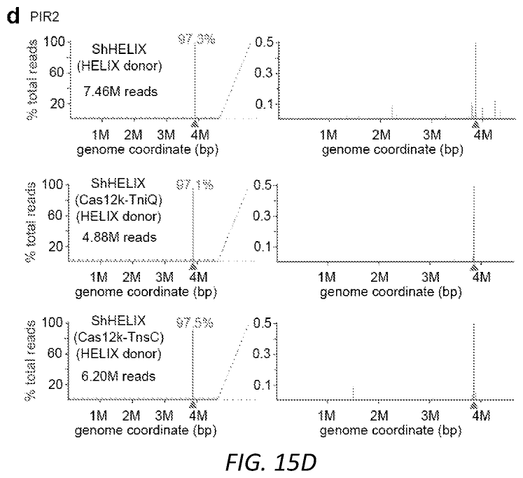

As shown herein, the constructs comprise flanking sequences, which are nucleotides directly adjacent to the LE and RE of the donor sequence to be inserted, e.g., on the donor plasmid (one example of which is referred to herein as pDonor), and which can influence integration. The flanking sequences can be, e.g., about 10-100, 10-20, 10-50, 10-30, 12-100, 12-50, 12-30, or 25-50 nucleotides long, and can be varied to influence integration efficiency (FIG. 4c and FIG. 6b). As used herein, a modified flanking sequence has at least one variation with respect to the corresponding flanking sequences from the organism from which the transposon sequence was obtained. The flanking sequences can be varied to enhance transposition efficiencies. Exemplary flanking sequences and their source organisms are provided in Table A. The flanking sequences can also be modified to include an endonuclease recognition site, e.g., an I-AniI site, on the 5′ and/or 3′ end, e.g., 4-50, 4-25, 10-20, 12-20, 4-15, 10-15, 12-15, 10-16, 10-16, or 10-18 nt away from the end of the sequence to be inserted. See additional exemplary sequence below and in FIG. 15.

TABLE A
EXEMPLARY 25 nt FLANKING SEQUENCES

| LE flanking sequence | RE flanking sequence | Source organism |
|---|---|---|
| TTAGACATCTCCACAAAA | CGTAGAGACGTAGCAATG | Scytonema |
| GGCGTAG (SEQ ID NO: 10) | CTACCTC (SEQ ID NO: 13) | hofmanni (UTEX |
| | | B 2349) |
| CGAGTCTCCTATTCTCCAT | ATAGCCTTTCTCACTCTA | Anabaena |
| TATATA (SEQ ID NO: 11) | GTTAGAT (SEQ ID NO: 14) | cylindrica (PCC |
| | | 7122) |
| ACTACCTACTTAAATGAAC | CCAACCCCAAGCATTGGT | Scytonema |
| CGCAAA (SEQ ID NO: 12) | ACCGAGC (SEQ ID NO: 15) | hofmannii (PCC |
| | | 7110) |

HE-Assisted Large-Sequence Integrating CAST compleX (HELIX)

Described herein are compositions and systems that can be used for programmable insertion of up to multi-kilobase DNA sequences into DNA, e.g., into the genome of a cell. The HELIX system component(s) include a fusion protein as described herein, e.g., comprising a transposon, e.g., TnsB, fused to a protein (such as, a nickase), optionally via an intervening linker. In some embodiments, a DNA cleavase fusion can be used instead of a nickase fusion for cut-and-paste DNA insertion.

Other HELIX system component(s) include cas12k, TnsC, and TniQ. A functional system comprises the TnsB-nickase fusion proteins, cas12k, TnsC, TniQ, and a guide RNA (e.g., a single guide RNA (sgRNA)) that binds to cas12k and directs the HELIX system to the intended insertion site, as well as a donor nucleic acid, e.g., a donor plasmid, comprising a sequence to be inserted that is preferably flanked by LE and RE sequences on the 5′ and 3′ ends, respectively, and a target site for the nickase (e.g., I-AniI), preferably oriented to confer a 5′ nick on the donor plasmid. The Cas12k enzyme itself is catalytically inactive; it binds the gRNA and is directed to bind the target site (but does not cleave or nick). Bound Cas12k recruits the downstream transposition machinery (such as TniQ, TnsC, and TnsB/nAniI-TnsB).

Coexpression of certain bacterial proteins (that is, host factors) along with the canonical CAST components can alter activity in bacteria or can rescue and improve activity in eukaryotic cells. Accordingly, in some embodiments also included are host factors that are known to alter DNA topology to increase insertion efficiency or specificity in prokaryotic or eukaryotic cells. For example, ribosomal protein S15 is required for type V-K CAST integration, ribosomal protein L29 (and host acyl carrier protein ACP) is required for efficient TnsD-mediated Tn7 transposition, and DnaN is required for efficient TnsE-mediated Tn7 transposition. DnaA, DNA topoisomerase I, La protease, and Dam methylase alter Tn5 transposition (Schmitz, M., Querques, I., Oberli, S., Chanez, C., & Jinek, M. (2022). Structural basis for RNA-mediated assembly of type V CRISPR-associated transposons. Biorxiv; Chandler, M., and Mahillon, J. (2002) Insertion sequences revisited. In Mobile DNA II, Vol. II. Craig, N. L., Craigie, R., Gellert, M., and Lambowitz, A. M. (eds). Washington, DC: American Society for Microbiology Press, pp. 305-366; Craig, N. L., Craigie, R., Gellert, M., and Lambowitz, A. M. (2002) Mobile DNA II. Washington, DC: American Society for Microbiology; Nagy, Z., and Chandler, M. (2004) Regulation of transposition in bacteria. Res Microbiol 155:387-398; Sharpe, P. L. & Craig, N. L. Host proteins can stimulate Tn7 transposition: a novel role for the ribosomal protein L29 and the acyl carrier protein. EMBO J. 17, 5822-5831 (1998); Parks, A. R. et al. Transposition into replicating DNA occurs through interaction with the processivity factor. Cell 138, 685-695 (2009). Furthermore, the nucleoid-associated proteins (NAPs) HU and IHF are required for efficient Mu transposition, and the same and/or other NAPs and DNA-bending proteins are a transposition requisite or enhancement for other transposon families (e.g. Tn10, IS903, Tn552, Sleeping Beauty, etc). Other examples of NAPS are H—NS, Fis, and TF1. Pi protein also alters DNA topology.

In other embodiments, the host factors are involved in DNA or cellular metabolism, proteolysis or protein folding, regulation, transport, and unknown functions in prokaryotic or eukaryotic cells. Examples proteins being: acyl carrier protein (ACP), Sigma S, or proteins expressed from the genes dcd, dinD, radA, recQ, clpX, fkpA, hflX, crl, rseB, rsxE, araJ, melB, mgtA, aspA, treC, proY, serA, yhbC, yidA, ykfA.

### Delivery and Expression Systems

To use the HELIX system described herein, it may be desirable to express one or more of the components from a nucleic acid that encodes them. This can be performed in a variety of ways. For example, a nucleic acid encoding a HELIX system component(s) can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the HELIX system component(s) for production of the HELIX system component(s). The nucleic acid encoding the HELIX system component(s) can also be cloned into an expression vector, for administration to a plant cell, animal cell, preferably a mammalian cell or a human cell, fungal cell, bacterial cell, or protozoan cell.

In some embodiments, a single expression vector is used that comprises sequences encoding a TnsB-nickase fusion protein, cas12k, TnsC, TniQ, and a single guide RNA that binds to cas12k. CASTs and their component parts are described in the art, see, e.g., Strecker et al., Science. 2019 Jul. 5; 365(6448):48-53; Rybarski et al., PNAS Dec. 7, 2021 118 (49) e2112279118; and US20200190487.

To obtain expression, a sequence encoding a HELIX system component(s) is typically subcloned into an expression construct, such as a vector, that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the proteins are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In some embodiments, e.g., when the HELIX system component(s) is to be expressed in vivo, either a constitutive or an inducible promoter can be used, depending on the particular use of the HELIX system component(s). In addition, a preferred promoter for administration of the HELIX system component(s) can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains other regulatory elements such as a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the HELIX system component(s), and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the HELIX system component(s), e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. Naked DNA and viral vectors (e.g., AAV), preferably non-integrative, can also be used.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors (e.g., AAV), both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the HELIX system component(s).

Alternatively, the methods can include delivering the HELIX system component(s) protein and guide RNA together, e.g., as a complex. For example, the HELIX system component(s) and gRNA can be can be overexpressed in a host cell and purified, then complexed with the guide RNA (e.g., in a test tube) to form a ribonucleoprotein (RNP), and delivered to cells. In some embodiments, the variant Cas9 can be expressed in and purified from bacteria through the use of bacterial Cas9 expression plasmids. For example, His-tagged variant Cas9 proteins can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

Thus, provided herein are the HELIX system component(s) (proteins and nucleic acids), vectors, and cells comprising the vectors.

### Methods of Use of the HELIX System

Provided herein are methods for inserting a desired sequence into DNA, e.g., into genomic DNA of a living cell, e.g., eukaryotic cell, e.g., a mammalian cell such as a cell from a human or non-human animal. The methods include expressing in the cell a nucleic acid sequence encoding a TnsB-nickase fusion protein as described herein; nucleic acid sequences encoding a TnsB-nickase fusion protein, cas12k, TnsC, TniQ, and a guide RNA that binds to cas12k; and a donor DNA molecule (e.g. a plasmid or linear dsDNA) comprising the desired sequence to be inserted, wherein the desired sequence is flanked by LE and RE sequences on the 5′ and 3′ ends, respectively, and a target site for the nickase (e.g., I-AniI), preferably oriented to confer a 5′ nick on the donor plasmid.

### EXAMPLES

The invention is further described in the following examples, which do not limit the scope of the invention described in the claims.

### Methods

The following materials and methods were used in the Examples below.

### Plasmids and Oligonucleotides

All plasmids used in this study and selected sequences are listed in Table 1. New plasmids were generated via isothermal assembly or Golden Gate assembly, some of which have been deposited with Addgene (Table 1). pHelper and pDonor plasmids for ShCAST and AcCAST, as well as pTarget, were gifts from Feng Zhang (Addgene plasmid numbers 127921, 127924, 127923, 127925, 127926). For gRNA-encoding plasmids, spacer sequences were cloned into pCAST and pHELIX plasmids via Golden Gate assembly with SapI (New England Biolabs, NEB). Target site features for all gRNAs used in this study are found in Supplementary Table 2. Oligonucleotides and probes used in this study were purchased from Integrated DNA Technologies (IDT) and are listed in Supplementary Table 3. Gene fragments for construct cloning were ordered from Twist Biosciences; synthetic SpCas9 sgRNAs were ordered from Synthego (Supplementary Table 2).

TABLE 1
Plasmids used in this study

| | | | |
|---|---|---|---|
| | | plasmid | |
| plasmid ID | Addgene ID | description | plasmid use |

CAST and HELIX Expression Plasmids; Parentheses in plasmid
description denote CAST ortholog

| | | | |
|---|---|---|---|
| pHelper_ | 127921 | (Sh) pLac-TnsB- | ShCAST |
| ShCAST_ | | TnsC-TniQ-cas12k- | experiments |
| sgRNA | | rrnB_Term-J23119- | |
| | | sgRNA_scaffold- | |
| | | (SapI)spacer_dropout | |
| | | (SapI)-term | |
| CJT30 | 181781 | (Sh) pLac- | Y2 ShHELIX |
| | | Y2nAniI(K227M)_ | plasmid-targeting |
| | | XTEN_TnsB- | experiments |
| | | TnsC-TniQ- | |
| | | Cas12k- | |
| | | rrnB_Term-J23119- | |
| | | sgRNA_scaffold- | |
| | | (SapI)spacer_dropout | |
| | | (SapI)-term | |
| CJT32 | 181782 | (Sh) pLac- | ShHELIX |
| | | nAniI(K227M)_XT | experiments |
| | | EN_TnsB-TnsC- | |
| | | TniQ-Cas12k- | |
| | | rrnB_Term-J23119- | |
| | | sgRNA_scaffold- | |
| | | (SapI)spacer_dropout | |
| | | (SapI)-term | |
| CJT57 | NA | (Sh) pLac- | ShHELIX linker |
| | | nAniI(K227M)_32a | length comparison |
| | | aXTEN_TnsB- | |
| | | TnsC-TniQ- | |
| | | Cas12k- | |
| | | rrnB_Term-J23119- | |
| | | sgRNA_scaffold- | |
| | | (SapI)spacer_dropout | |
| | | (SapI)-term | |
| CJT77 | NA | (Sh) pLac- | ShHELIX plasmid- |
| | | dAniI(K227M, | targeting |
| | | Q171K)_XTEN_ | experiments control |
| | | TnsB-TnsC-TniQ- | |
| | | Cas12k- | |
| | | rrnB_Term-J23119- | |
| | | sgRNA_scaffold- | |
| | | (SapI)spacer_dropout | |
| | | (SapI)-term | |
| CJT82 | NA | (Ac) pLac-TnsB- | AcCAST sgRNA |
| | | TnsC-TniQ-cas12k- | testing |
| | | rrnB_Term-J23119- | |
| | | sgRNA_scaffold_1- | |
| | | (SapI)spacer_dropout | |
| | | (SapI)-term | |
| CJT83 | 181785 | (Ac) pLac-TnsB- | AcCAST |
| | | TnsC-TniQ-cas12k- | experiments |
| | | rrnB_Term-J23119- | |
| | | sgRNA_scaffold_2- | |
| | | (SapI)spacer_dropout | |
| | | (SapI)-term | |
| CJT94 | 181783 | (Ac) pLac- | AcHELIX |
| | | nAniI(K227M)_XT | experiments |
| | | EN_TnsB-TnsC- | |
| | | TniQ-Cas12k- | |
| | | rrnB_Term-J23119- | |
| | | sgRNA_scaffold- | |
| | | (SapI)spacer_dropout | |
| | | (SapI)-term | |
| BO1 | 181786 | (Sho) pLac-TnsB- | ShoCAST |
| | | TnsC-TniQ-cas12k- | experiments |
| | | rrnB_Term-J23119- | |
| | | sgRNA_scaffold- | |
| | | (SapI)spacer_dropout | |
| | | (SapI)-term | |
| BO3 | 181784 | (Sho) pLac- | ShoHELIX |
| | | nAniI(K227M)_XT | experiments |
| | | EN_TnsB-TnsC- | |
| | | TniQ-Cas12k- | |
| | | rrnB Term-J23119- | |
| | | sgRNA_scaffold- | |
| | | (SapI)spacer_dropout | |
| | | (SapI)-term | |
| CJT10 | NA | (Sh) pLac-TnsB- | 3-component |
| | | TnsC- | ShCAST |
| | | TniQ_XTEN_Cas1 | experiments (TniQ- |
| | | 2k-rrnB_Term- | Cas12k) |
| | | J23119- | |
| | | sgRNA_scaffold- | |
| | | (SapI)spacer_dropout | |
| | | (SapI)-term | |
| CJT11 | 181787 | (Sh) pLac-TnsB- | 3-component |
| | | TnsC- | ShCAST |
| | | Cas12k_XTEN_TniQ- | experiments |
| | | rrnB_Term- | (Cas12k-TniQ) |
| | | J23119- | |
| | | sgRNA_scaffold- | |
| | | (SapI)spacer_dropout | |
| | | (SapI)-term | |
| CJT12 | 181788 | (Sh) pLac-TnsB- | 3-component |
| | | TnsC- | ShCAST |
| | | Cas12k_XTEN_TniQ_ | experiments |
| | | GGGS(x3) (SEQ | (Cas12k-TniQ- |
| | | ID NO: 157)_TniQ- | TniQ) |
| | | rrnB_Term-J23119- | |
| | | sgRNA_scaffold- | |
| | | (SapI)spacer_dropout | |
| | | (SapI)-term | |
| CJT13 | NA | (Sh) pLac-TnsB- | 3-component |
| | | TnsC- | ShCAST |
| | | TniQ_GGGS(x3) | experiments (TniQ- |
| | | (SEQ ID | TniQ-Cas12k) |
| | | NO: 157)_TniQ_XT | |
| | | EN_Cas12k- | |
| | | rrnB_Term-J23119- | |
| | | sgRNA_scaffold- | |
| | | (SapI)spacer_dropout | |
| | | (SapI)-term | |
| CJT14 | NA | (Sh) pLac-TnsB- | 3-component |
| | | TnsC- | ShCAST |
| | | TniQ_XTEN_Cas12k_ | experiments (TniQ- |
| | | XTEN_TniQ- | Cas12k-TniQ) |
| | | rrnB_Term-J23119- | |
| | | sgRNA_scaffold- | |
| | | (SapI)spacer_dropout | |
| | | (SapI)-term | |
| CJT27 | NA | (Sh) pLac-TnsB- | 3-component |
| | | TniQ- | ShCAST |
| | | TnsC_XTEN_cas12k- | experiments (TnsC- |
| | | rrnB_Term- | Cas12k) |
| | | J23119- | |
| | | sgRNA_scaffold- | |
| | | (SapI)spacer_dropout | |
| | | (SapI)-term | |
| CJT28 | 181789 | (Sh) pLac-TnsB- | 3-component |
| | | TniQ- | ShCAST |
| | | cas12k_XTEN_TnsC- | experiments |
| | | rrnB_Term- | (Cas12k-TnsC) |
| | | J23119- | |
| | | sgRNA_scaffold- | |
| | | (SapI)spacer_dropout | |
| | | (SapI)-term | |
| CJT111 | 181790 | (Sh) pLac- | 3-component |
| | | nAniI(K227M)_XT | ShHELIX |
| | | EN_TnsB-TnsC- | experiments |
| | | Cas12k_XTEN_TniQ- | (Cas12k-TniQ) |
| | | rrnB_Term- | |
| | | J23119- | |
| | | sgRNA_scaffold- | |
| | | (SapI)spacer_dropout | |
| | | (SapI)-term | |
| CJT112 | 181791 | (Sh) pLac- | 3-component |
| | | nAniI(K227M)_XT | ShHELIX |
| | | EN_TnsB-TnsC- | experiments |
| | | Cas12k_XTEN_TniQ_ | (Cas12k-TniQ- |
| | | GGGS(x3) (SEQ | TniQ) |
| | | ID NO: 157)_TniQ- | |
| | | rrnB_Term-J23119- | |
| | | sgRNA_scaffold- | |
| | | (SapI)spacer_dropout | |
| | | (SapI)-term | |
| CJT113 | 181792 | (Sh) pLac- | 3-component |
| | | nAniI(K227M)- | ShHELIX |
| | | TniQ- | experiments |
| | | cas12k_XTEN_TnsC- | (Cas12k-TnsC) |
| | | rrnB_Term- | |
| | | J23119- | |
| | | sgRNA_scaffold- | |
| | | (SapI)spacer_dropout | |
| | | (SapI)-term | |
| CJT169 | NA | (Sh) pLac-TnsB- | 2-component |
| | | Cas12k_XTEN_TniQ_ | ShCAST |
| | | GGGS(x3) (SEQ | experiments |
| | | ID NO: 157)_TnsC- | (Cas12k-TniQ- |
| | | rrnB_Term-J23119- | TnsC) |
| | | sgRNA_scaffold- | |
| | | (SapI)spacer_dropout | |
| | | (SapI)-term | |
| CJT170 | NA | (Sh) pLac-TnsB- | 2-component |
| | | cas12k_XTEN_TnsC_ | ShCAST |
| | | GGGS(x3) (SEQ | experiments |
| | | ID NO: 157)_TniQ- | (Cas12k-TnsC- |
| | | rrnB_Term-J23119- | TniQ) |
| | | sgRNA_scaffold- | |
| | | (SapI)spacer_dropout | |
| | | (SapI)-term | |
| CJT195 | NA | (Sh) pLac- | TnsA fusion |
| | | TnsA(E. coli, N- | experiments |
| | | term- | |
| | | dom)_XTEN_TnsB- | |
| | | TnsC-TniQ- | |
| | | cas12k-rrnB_Term- | |
| | | J23119- | |
| | | sgRNA_scaffold- | |
| | | (SapI)spacer_dropout | |
| | | (SapI)-term | |
| CJT196 | NA | (Sh) pLac-TnsA(N. | TnsA fusion |
| | | Punctiforme, N- | experiments |
| | | term- | |
| | | dom)_XTEN_TnsB- | |
| | | TnsC-TniQ- | |
| | | cas12k-rrnB_Term- | |
| | | J23119- | |
| | | sgRNA_scaffold- | |
| | | (SapI)spacer_dropout | |
| | | (SapI)-term | |
| CJT197 | NA | (Sh) pLac- | TnsA fusion |
| | | TnsA(Ripkkae, N- | experiments |
| | | term- | |
| | | dom) GSG_XTEN- | |
| | | TnsC-TniQ- | |
| | | cas12k-rrnB_Term- | |
| | | J23119- | |
| | | sgRNA_scaffold- | |
| | | (SapI)spacer_dropout | |
| | | (SapI)-term | |
| CJT198 | NA | (Sh) pLac-TnsA(A. | TnsA fusion |
| | | Wodanis, N-term- | experiments |
| | | dom)_XTEN_TnsB- | |
| | | TnsC-TniQ- | |
| | | cas12k-rrnB_Term- | |
| | | J23119- | |
| | | sgRNA_scaffold- | |
| | | (SapI)spacer_dropout | |
| | | (SapI)-term | |
| CJT165 | 160731 | VchINTEGRATE | INTEGRATE/ |
| | | pEffector | HELIX comparison |
| CJT201 | NA | VchINTEGRATE | INTEGRATE/ |
| | | pSpin w/2.1 kb | HELIX comparison |
| | | cargo (based off | |
| | | addgene #160730) | |
| CJT228 | 190661 | (N7) pCMV- | N7CAST |
| | | Cas12k-NLS-T2A- | experiments |
| | | TnsC-IRES- | |
| | | NLS_TniQ-T2A- | |
| | | NLS_TnsB | |
| CJT248 | 190662 | (N7) pCMV- | N7HELIX |
| | | Cas12k-NLS-T2A- | experiments |
| | | TnsC-IRES- | |
| | | NLS_TniQ-T2A- | |
| | | NLS_nAniI_TnsB | |
| CJT230 | 190664 | (N7) pU6- | N7CAST/HELIX |
| | | N7sgRNA2 | experiments |

Donor Plasmids

| | | | |
|---|---|---|---|
| pDonor_ | 127924 | LE(ShCAST)- | ShCAST |
| ShCAST_ | | KanR- | experiments |
| kanR | | RE(ShCAST) | |
| CJT37 | NA | I-AniI_site-4 bp- | I-AniI site to |
| | | LE(ShCAST)- | LE/RE spacing |
| | | KanR- | experiments |
| | | RE(ShCAST)-4 bp- | |
| | | I-AniI_site | |
| CJT38 | NA | I-AniI_site-6 bp- | I-AniI site to |
| | | LE(ShCAST)- | LE/RE spacing |
| | | KanR- | experiments |
| | | RE(ShCAST)-6 bp- | |
| | | I-AniI_site | |
| CJT39 | NA | I-AniI_site-8 bp- | I-AniI site to |
| | | LE(ShCAST)- | LE/RE spacing |
| | | KanR- | experiments |
| | | RE(ShCAST)-8 bp- | |
| | | I-AniI_site | |
| CJT40 | NA | I-AniI_site-10 bp- | I-AniI site to |
| | | LE(ShCAST)- | LE/RE spacing |
| | | KanR- | experiments |
| | | RE(ShCAST)- | |
| | | 10 bp-I-AniI_site | |
| CJT41 | NA | I-AniI_site-12 bp- | I-AniI site to |
| | | LE(ShCAST)- | LE/RE spacing |
| | | KanR- | experiments |
| | | RE(ShCAST)- | |
| | | 12 bp-I-AniI_site | |
| CJT74 | NA | I-AniI_site-13 bp- | I-AniI site to |
| | | LE(ShCAST)- | LE/RE spacing |
| | | KanR- | experiments (CFU |
| | | RE(ShCAST)- | counting only) |
| | | 13 bp-I-AniI_site | |
| CJT70 | 181793 | I-AniI_site-14 bp- | I-AniI site to |
| | | LE(ShCAST)- | LE/RE spacing |
| | | KanR- | experiments (main |
| | | RE(ShCAST)- | ShHELIX donor) |
| | | 14 bp-I-AniI_site | |
| CJT75 | NA | I-AniI_site-15 bp- | I-AniI site to |
| | | LE(ShCAST)- | LE/RE spacing |
| | | KanR- | experiments (CFU |
| | | RE(ShCAST)- | counting only) |
| | | 15 bp-I-AniI_site | |
| CJT71 | NA | I-AniI_site-16 bp- | I-AniI site to |
| | | LE(ShCAST)- | LE/RE spacing |
| | | KanR- | experiments |
| | | RE(ShCAST)- | |
| | | 16 bp-I-AniI_site | |
| CJT72 | NA | I-AniI_site-18 bp- | I-AniI site to |
| | | LE(ShCAST)- | LE/RE spacing |
| | | KanR- | experiments |
| | | RE(ShCAST)- | |
| | | 18 bp-I-AniI_site | |
| CJT73 | NA | flipped_I-AniI_site- | ShHELIX plasmid- |
| | | 14 bp- | targeting control |
| | | LE(ShCAST)- | |
| | | KanR- | |
| | | RE(ShCAST)- | |
| | | 14 bp-I-flipped_I- | |
| | | AniI_site | |
| CJT76 | NA | Lib4_I-AniI_site- | ShHELIX plasmid- |
| | | 14 bp- | targeting control |
| | | LE(ShCAST)- | |
| | | KanR- | |
| | | RE(ShCAST)- | |
| | | 14 bp-Lib4_I- | |
| | | AniI_site | |
| pDonor_ | 127925 | LE(AcCAST)- | AcCAST flank |
| AcCAST_ | | KanR- | comparison |
| kanR | | RE(AcCAST) with | |
| | | “native flanks” | |
| CJT84 | NA | LE(AcCAST)- | AcCAST flank |
| | | KanR- | comparison |
| | | RE(AcCAST) with | |
| | | “ShCAST flanks” | |
| CJT96 | 181794 | I-AniI_site-14 bp- | AcCAST/HELIX |
| | | LE(AcCAST)- | experiments |
| | | KanR- | |
| | | RE(AcCAST)- | |
| | | 14 bp-I-AniI_site | |
| | | with “ShCAST | |
| | | flanks” | |
| BO2 | NA | LE(ShoCAST)- | ShoCAST |
| | | KanR- | experiments |
| | | RE(ShoCAST) with | |
| | | “ShCAST flanks” | |
| BO4 | 181795 | I-AniI_site-14 bp- | ShoCAST/HELIX |
| | | LE(ShoCAST)- | experiments |
| | | KanR- | |
| | | RE(ShoCAST)- | |
| | | 14 bp-I-AniI_site | |
| | | with “ShCAST | |
| | | flanks” | |
| BO5 | NA | I-AniI_site-14 bp- | ShHELIX cargo |
| | | LE(ShCAST)-4.8 kb | size comparisons |
| | | stuffer (includes | |
| | | KanR)- | |
| | | RE(ShCAST)- | |
| | | 14 bp-I-AniI_site | |
| BO6 | NA | I-AniI_site-14 bp- | ShHELIX cargo |
| | | LE(ShCAST)-7.3 kb | size comparisons |
| | | stuffer (includes | |
| | | KanR)- | |
| | | RE(ShCAST)- | |
| | | 14 bp-I-AniI_site | |
| BO14 | NA | I-AniI_site-14 bp- | ShHELIX cargo |
| | | LE(ShCAST)-9.3 kb | size comparisons |
| | | stuffer (includes | |
| | | KanR)- | |
| | | RE(ShCAST)- | |
| | | 14 bp-I-AniI_site | |
| BO10 | NA | I-AniI_site-14 bp- | AcHELIX cargo |
| | | LE(AcCAST)- | size comparisons |
| | | 4.8 kb stuffer | |
| | | (includes KanR)- | |
| | | RE(AcCAST)- | |
| | | 14 bp-I-AniI_site | |
| BO11 | NA | I-AniI_site-14 bp- | AcHELIX cargo |
| | | LE(AcCAST)- | size comparisons |
| | | 7.3 kb stuffer | |
| | | (includes KanR)- | |
| | | RE(AcCAST)- | |
| | | 14 bp-I-AniI_site | |
| BO9 | NA | I-AniI site-14 bp- | AcHELIX cargo |
| | | LE(AcCAST)- | size comparisons |
| | | 9.3 kb stuffer | |
| | | (includes KanR)- | |
| | | RE(AcCAST)- | |
| | | 14 bp-I-AniI_site | |
| BO7 | NA | I-AniI_site-14 bp- | ShoHELIX cargo |
| | | LE(ShoCAST)- | size comparisons |
| | | 4.8 kb stuffer | |
| | | (includes KanR)- | |
| | | RE(ShoCAST)- | |
| | | 14 bp-I-AniI_site | |
| BO8 | NA | I-AniI_site-14 bp- | ShoHELIX cargo |
| | | LE(ShoCAST)- | size comparisons |
| | | 7.3 kb stuffer | |
| | | (includes KanR)- | |
| | | RE(ShoCAST)- | |
| | | 14 bp-I-AniI_site | |
| BO13 | NA | I-AniI_site-14 bp- | ShoHELIX cargo |
| | | LE(ShoCAST)- | size comparisons |
| | | 9.3 kb stuffer | |
| | | (includes KanR)- | |
| | | RE(ShoCAST)- | |
| | | 14 bp-I-AniI_site | |
| CJT231 | 190666 | I-AniI_site-14 bp- | ShCAST/HELIX |
| | | LE(ShCAST)- | specificity |
| | | KanR- | experiments in non- |
| | | RE(ShCAST)- | pir cells |
| | | 14 bp-I-AniI_site on | |
| | | temperature | |
| | | sensitive SC101 | |
| | | origin | |
| CJT221 | 190663 | I-AniI_site-14 bp- | N7CAST/HELIX |
| | | LE(N7CAST)- | experiments |
| | | KanR- | |
| | | RE(N7CAST)- | |
| | | 14 bp-I-AniI_site | |
| CJT202 | NA | RE(VchINT)-2.1 kb | INTEGRATE/ |
| | | stuffer- | HELIX comparison |
| | | LE(VchINT) | |

Other Plasmids

| | | | |
|---|---|---|---|
| pTarget_ | 127926 | pTarget containing | plasmid-targeting |
| CAST | | TS1 | experiments |
| pPir_wt | 190660 | Pi protein | ShCAST/HELIX |
| | | expressed from | specificity |
| | | endogenous | experiments |
| | | promoter found in | |
| | | PIR2 cells (thermo | |
| | | fischer) | |
| pPir116 | NA | Pi protein copy- | ShCAST/HELIX |
| | | number mutant | specificity |
| | | expressed from | experiments |
| | | endogenous | |
| | | promoter found in | |
| | | PIR1 cells (thermo | |
| | | fischer) | |
| pN7_S15 | 190665 | pCMV-N7S15 | N7CAST/HELIX |
| | | | plasmid targeting |
| | | | experiments |

TABLE 2
gRNAs used in this study

| For transposition experiments | | | |
|---|---|---|---|
| site name | 5′ PAM (NGTN) | spacer sequence | target molecule |
| TS1 | GGTT | GAGAAGTCATTTAATAAG | plasmid |
| | | GCCAC (SEQ ID NO: 16) | |
| TS2 | AGTT | ATAGCGATCCCTTGCTGAA | genome |
| | | AATA (SEQ ID NO: 17) | |
| TS3 | CGTT | ATAGTGAATCCGCTTATTC | genome |
| | | TCAG (SEQ ID NO: 18) | |
| TS4 | AGTC | ACTGCCCGTTTCGAGAGTT | genome |
| | | TCTC (SEQ ID NO: 19) | |
| TS5 | CGTT | ACCACCTCAAGCTATGCCG | genome |
| | | CCAG (SEQ ID NO: 20) | |
| TS6 | AGTG | ACTATAGACTATCCGGGCA | genome |
| | | ATGT (SEQ ID NO: 21) | |
| TS7 | TGTT | ACCCTCTTAAACTATCCCA | genome |
| | | CTAA (SEQ ID NO: 22) | |

For Cas9-enrichment nanopore sequencing library prep

| site name | spacer sequence | 3′ PAM (NGGN) | target molecule |
|---|---|---|---|
| TS2 | TAGTATAAACGAACAG | AGGC | genome |
| upstream | GATC (SEQ ID NO: 23) | | |
| 1 | | | |
| TS2 | GAATATCAAACAGTTT | AGGA | genome |
| upstream | ATGC (SEQ ID NO: 24) | | |
| 2 | | | |
| TS2 | TGCTCACCAATACCAA | TGGA | genome |
| downstre | TACC (SEQ ID NO: 25) | | |
| am 1 | | | |
| TS2 | TTCACTCACATTCATCA | TGGC | genome |
| downstre | CGA (SEQ ID NO: 26) | | |
| am 2 | | | |

TABLE 3
Oligonucleotides and probes used in this study
ddPCR primers

| primer ID | primer description | primer sequence |
|---|---|---|
| oCT39 | ShCAST insert primer | AACGCTGATGGGTCAC |
| | binding LE | GACG (SEQ ID NO: 27) |
| oCT390 | genome control forward | CGCGGCAACTTTGTAG |
| | primer | TACCAGC (SEQ ID |
| | | NO: 28) |
| oCT391 | genome control reverse | CCCTTTTCAGATTTCT |
| | primer | GCCCGACGC (SEQ ID |
| | | NO: 29) |
| oCT392 | pTarget control forward | CGACAGCATCGCCAGT |
| | primer | CACTATG (SEQ ID |
| | | NO: 30) |
| oCT393 | pTarget control reverse | CAAGTAGCGAAGCGA |
| | primer | GCAGGAC (SEQ ID |
| | | NO: 31) |
| oCT394 | pTarget primer upstream of | AGTCATTTAATAAGGC |
| | insertion site (TS1) | CACTGTTAAACG (SEQ |
| | | ID NO: 32) |
| oCT417 | ShoCAST insert primer | GTTCCTATAATTGAAT |
| | binding LE | TGATGAGACAAACTAT |
| | | TC (SEQ ID NO: 33) |
| oCT453 | AcCAST insert primer | GAAAACTTAGAATAAT |
| | binding LE | TAAATTGACTCTG |
| | | (SEQ ID NO: 34) |
| oCT839 | N7CAST insert primer | TTTCGCAATTAGCATT |
| | binding LE | ATACGACAC (SEQ ID |
| | | NO: 35) |
| oCT797 | VchINT insert primer | CGAGGAAAATGTCGT |
| | binding RE | AAACTTACTG (SEQ ID |
| | | NO: 36) |
| oCT82 | TS2 primer to assess RL- | GTCAGGTAGCCAGAA |
| | oriented insertions | CACCC (SEQ ID NO: 37) |
| oCT83 | TS2 primer to assess LR- | GCCGGGATACGTTCCT |
| | oriented insertions | TCTT (SEQ ID NO: 38) |
| 0CT78 | TS3 primer to assess RL- | ACGTTCGAAAGGCGTA |
| | oriented insertions | CCAA (SEQ ID NO: 39) |
| oCT79 | TS3 primer to assess LR- | TGAGTGCCATTGTAGT |
| | oriented insertions | GCGA (SEQ ID NO: 40) |
| oCT80 | TS4 primer to assess RL- | GCAGGCTCGGTTAGGG |
| | oriented insertions | TAAG (SEQ ID NO: 41) |
| oCT81 | TS4 primer to assess LR- | GGCTAACGTGGCAGG |
| | oriented insertions | AATCT (SEQ ID NO: 42) |
| oCT86 | TS5 primer to assess RL- | TTGGTAGGCCTGATAA |
| | oriented insertions | GCGC (SEQ ID NO: 43) |
| oCT87 | TS5 primer to assess LR- | GTAGCAGATGACCTCG |
| | oriented insertions | CCTC (SEQ ID NO: 44) |
| oCT88 | TS6 primer to assess RL- | TGAGTGCCAGAATCTT |
| | oriented insertions | GCGT (SEQ ID NO: 45) |
| 0CT89 | TS6 primer to assess LR- | ACGTACTTCGCCACCT |
| | oriented insertions | GAAG (SEQ ID NO: 46) |
| oCT495 | TS7 primer to assess RL- | AAGGCTGGGAAATCA |
| | oriented insertions | GACGG (SEQ ID NO: 47) |
| oCT496 | TS7 primer to assess LR- | TATCTGCAAAGTCGCT |
| | oriented insertions | GGGG (SEQ ID NO: 48) |
| oCT828 | Target immunity primer | GCATGAGCTCACTAGT |
| | binding just interior of | GGATCC (SEQ ID |
| | ShCAST LE | NO: 49) |

ddPCR probes

| probe ID | probe description | probe sequence |
|---|---|---|
| prCT3 | ShCAST/HELIX insert | CTGTCGTCGGTGACAG |
| | probe (5′ FAM, 3′ Iowa | ATTAATGTCATTGTGA |
| | Black) | C (SEQ ID NO: 50) |
| prCT4 | pTarget control probe (5′ | TGCGTTGATGCAATTT |
| | FAM, 3′ Iowa Black) | CTATGCGCACCCGT |
| | | (SEQ ID NO: 51) |
| prCT5 | Genome control probe (5′ | ACGTTCGCGTTTGCCG |
| | FAM, 3′ Iowa Black) | TGCGTGTAATGTAGTA |
| | | C (SEQ ID NO: 52) |
| prCT8 | AcCAST/HELIX insert | TCGCAATTTAGTGTCG |
| | probe (5′ FAM, 3′ Iowa | TTATTCGCAAATTAAT |
| | Black) | GTC (SEQ ID NO: 53) |
| prCT9 | ShoCAST/HELIX insert | ATGTCGTAATTCGCAA |
| | probe (5′ FAM, 3′ Iowa | ATTTGTGTCGTTTTTCG |
| | Black) | C (SEQ ID NO: 54) |
| prCT19 | VchINTEGRATE insert | CACACCCATAAATTGA |
| | probe (5′ FAM, 3′ Iowa | TATTGCCTCTTCATGG |
| | Black) | TC (SEQ ID NO: 55) |
| prCT20 | N7CAST/HELIX insert | TCGTTGTTAACAGATT |
| | probe (5′ FAM, 3′ Iowa | GCTGTCGCTATTAAC |
| | Black) | (SEQ ID NO: 56) |

Primers for next-generation sequencing library prep

| primer ID | primer description | primer sequence |
|---|---|---|
| oCT552 | NGS universal reverse | GACTGGAGTTCAGACG |
| | primer for TS2 | TGTGCTCTTCCGATCT |
| | | TCATAATAAATTCATC |
| | | TGTTGATCGTGGG |
| | | (SEQ ID NO: 57) |
| oCT553 | NGS forward primer for | ACACTCTTTCCCTACA |
| | ShCAST/HELIX off of LE | CGACGCTCTTCCGATC |
| | | TCACAATGACATTAAT |
| | | CTGTCACCGAC (SEQ |
| | | ID NO: 58) |
| oCT554 | NGS forward primer for | ACACTCTTTCCCTACA |
| | AcCAST/HELIX off of LE | CGACGCTCTTCCGATC |
| | | TCCACGACATTAATTT |
| | | GCGAATAACGAC (SEQ |
| | | ID NO: 59) |
| oCT555 | NGS forward primer for | ACACTCTTTCCCTACA |
| | ShoCAST/HELIX off of | CGACGCTCTTCCGATC |
| | LE | TACAAACTATTCTAAA |
| | | CGACATTAATTTGCG |
| | | (SEQ ID NO: 60) |
| oCT846 | NGS universal forward | ACACTCTTTCCCTACA |
| | primer for TS1 | CGACGCTCTTCCGATC |
| | | TTCTACGATACGTAGT |
| | | ATCTACGATAC (SEQ |
| | | ID NO: 61) |
| oCT847 | NGS reverse primer for | GACTGGAGTTCAGACG |
| | N7CAST/HELIX off of LE | TGTGCTCTTCCGATCT |
| | | TTTCGCAATTAGCATT |
| | | ATACGACAC (SEQ ID |
| | | NO: 62) |

Primers for specificity analysis (genome-LE junction enrichment)

| primer ID | primer description | primer sequence |
|---|---|---|
| oCT141 | i7 specific primer (binds | GACTGGAGTTCAGACG |
| | stubby adaptor) | TGTGC (SEQ ID NO: 63) |
| oCT774 | Reverse primer with i5 | ACACTCTTTCCCTACA |
| | adaptor binding ShCAST | CGACGCTCTTCCGATC |
| | LE | TGTCACCGACGACAGA |
| | | TAATTTGTC (SEQ ID |
| | | NO: 64) |
| Stubby Adaptors | TA-ligation adaptors (IDT) | NA |

Primers for N7 lysate enrichment for nanopore sequencing library prep

| primer ID | primer description | primer sequence |
|---|---|---|
| oCT110 | Universal forward primer | TTCAGAGCAAGAGATT |
| | binding pTarget | ACGCGCAG (SEQ ID |
| | | NO: 65) |
| oCT935 | Reverse primer binding | TGTCGTCTTAACAAAA |
| | N7CAST RE (counts | TAATGTCGTC (SEQ ID |
| | “total) | NO: 66) |
| oCT34 | Reverse primer binding | TTGAGTGACACAGGA |
| | pDonor backbone (counts | ACACTTAAC (SEQ ID |
| | “cointegrates”) | NO: 67) |

### Transposition Assays Targeting Plasmids and Genomic Sites

Transformations for plasmid targeting experiments were performed in chemically competent PIR1 cells containing pTarget (original PIR1 strain obtained from Invitrogen), using 25 ng of pCAST or pHELIX and 25 ng of pDonor. For target-immunity experiments, 25 ng of pTarget encoding a pre-inserted mini transposon (containing a different cargo than pDonor) was cotransformed with pCAST or pHELIX and pDonor in PIR1 cells that did not harbor any plasmids. Transformed cells were recovered for 1 hr at 37° C. in S.O.C. and then plated on LB agar plates containing 50 μg/mL kanamycin, 25 μg/mL chloramphenicol, and 100 μg/mL carbenicillin. Plates were incubated at 37° C. for 18 hrs. Colonies were counted, scraped, and plasmid DNA extracted via miniprep (Qiagen). The resulting plasmid pool was used for downstream analysis via junction PCR and long-read sequencing. Junction PCRs were analyzed via QIAxcel Capillary Electrophoresis (Qiagen) and visualized with QIAxcel ScreenGel Software (v1.5.0.16; Qiagen).

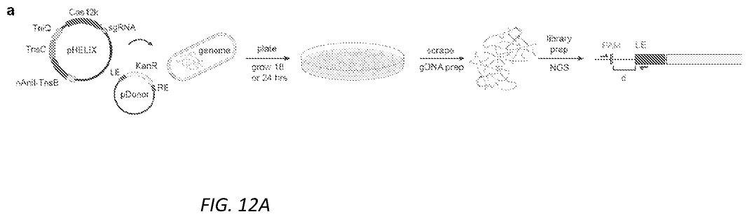

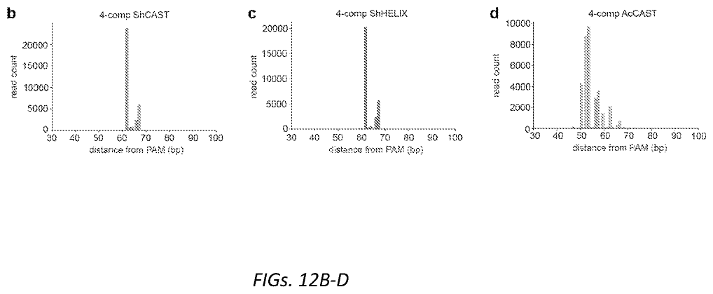

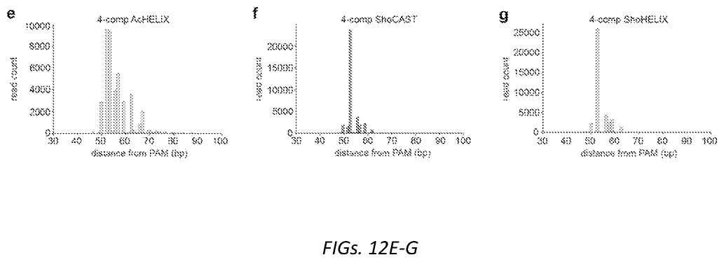

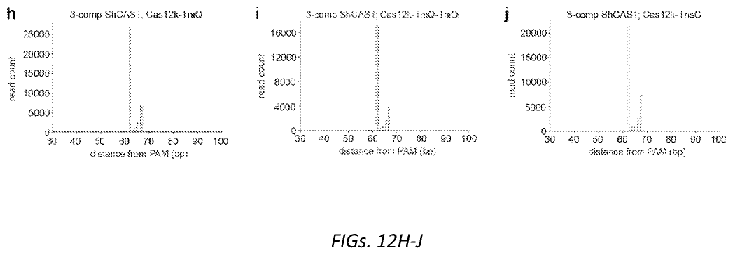

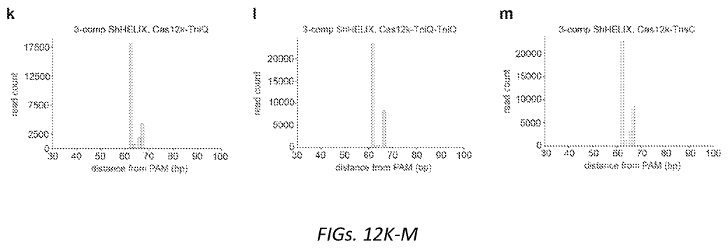

Transformations for genome targeting experiments were performed using PIR1 cells (or PIR2 cells (Invitrogen) for FIG. 12) and 25 ng of pCAST or pHELIX and 25 ng of pDonor. Transformed cells were recovered for 1 hr at 37° C. in S.O.C. and then plated on LB agar plates containing 50 μg/mL kanamycin and 100 μg/mL carbenicillin. For transformations including ShCAST, ShHELIX, ShoCAST, or ShoHELIX plasmids, plates were incubated at 37° C. for 18 hours; for AcCAST and AcHELIX transformations, plates were incubated at 37° C. for 24 hrs due to comparatively smaller colonies (though approximately the same in number). Colonies were scraped and gDNA was harvested using Wizard Genomic DNA Purification Kit (Promega) for downstream analysis via ddPCR and long-read sequencing.

Assessment of Integration Efficiency Via ddPCR

Plasmid or genomic DNA from E. coli transposition assays was normalized to 10 ng/μL or 100 ng/μL, respectively, and then further diluted to 0.2 ng/μL or 2 ng/μL working stocks, respectively. Extracted DNA (genome/plasmid mixture) from plasmid-targeting HEK293T transposition assays were used undiluted for insertion detection and 100-fold diluted to count total pTarget plasmids. Insertion events were measured using target-specific primers and a donor-specific probe (Supplementary Table 3). For target immunity experiments specifically, the reverse primer to detect insertions bound just interior of the LE on the cargo (which differed between the pre-installed insertion and the cargo to be inserted) instead of on the LE directly. ddPCR reactions contained 20 μg of plasmid DNA (from E. coli, plasmid-targeting assays), 2 ng E. coli gDNA, or 4 μL of gDNA/plasmid mixture (from HEK293T plasmid-targeting assays), 250 nM each primer, 900 nM probe, and ddPCR supermix for probes (no dUTP) (BioRad) in 20 μL reactions, and droplets were generated using a QX200 Automated Droplet Generator (BioRad). Thermal cycling conditions were: 1 cycle of (95° C. for 10 min), 40 cycles of (94° C. for 30 sec, 58° C. for 1 min), 1 cycle of (98° C. for 10 min), hold at 4° C. PCR products were analyzed using a QX200 Droplet Reader (BioRad) and absolute quantification of inserts was determined using QuantaSoft (v1.7.4). Total template DNA was also analyzed, and integration efficiencies were calculated by inserts/template*100.

### Long-Read Sequencing of Plasmid and Genomic Integrations

Integration product purity was analyzed via long-read sequencing using the plasmids resulting from plasmid targeting transposition reactions in E. coli (where HELIX pDonor was used for all conditions). Transposed products were enriched by electroporating approximately 100 ng of plasmid pool into Endura Electrocompetent Cells (Lucigen), which are a non-PIR strain that limits recombination. Cells were recovered for 1 hr at 37° C. in S.O.C. and spread on LB agar plates containing 50 μg/mL kanamycin and 25 μg/mL chloramphenicol. Plates were incubated at 30° C. (to limit recombination) for 24 hrs, scraped, and plasmid DNA extracted via miniprep. Enriched plasmids were digested with EcoRV (NEB) for 8 hrs at 37° C. Amplification-free long-read sequencing library preparation (Oxford Nanopore Technologies, SQK-LSK109) was performed using a barcode expansion kit (Oxford Nanopore Technologies, NBD-104). The final pooled library was loaded onto an R9.4.1 flow cell and sequenced for 24 hrs.

To conduct long-read sequencing of E. coli genome-targeted insertions, we performed an amplification-free Cas9 targeted enrichment protocol to improve sequencing selectively of the intended on-target sites (Oxford Nanopore Technologies, SQK-CS9109; sgRNAs listed in Supplementary Table 2). As described in the SQK-CS9109 protocol, normalized aliquots of genomic DNA from genome-targeting transposition assays (where HELIX pDonor was used for all conditions) were dephosphorylated, and Cas9 and gRNA RNPs were targeted to cleave approximately +/−1.5kb of the target site on the dephosphorylated gDNA according to the SQK-CS9109 protocol. Adaptors were selectively ligated to these segments, thereby enriching for the target region and increasing sensitivity of our sequencing on genomic targets. The resulting library was loaded onto an R9.4.1 flow cell and sequenced for 30 hrs.

To analyze the integration product purity from N₇CAST and N₇HELIX human lysate experiments (described below), a PCR-based enrichment strategy that minimizes size and template bias was employed due to low efficiency transposition (Example 11). Two sets of primers were used that either amplify from upstream of TS1 to the RE of the insertion product (irrespective of simple insertion or cointegrate) or upstream of TS1 to the backbone of cointegrates. These two reactions were performed in separate PCR reactions using Q5 High-fidelity DNA Polymerase (NEB) and containing identical volume of terminated lysate reaction as template (2 μL). Thermal cycling conditions for both PCRs were: 98° C. for 2 min followed by 20 cycles of (98° C. for 10 sec, 64° C. for 15 sec, 72° C. for 90 sec) and a final extension of 72° C. for 3 min. The two reactions were combined and purified with 1× AmpureXP beads. Amplification-free long-read sequencing library preparation (Oxford Nanopore Technologies, SQK-LSK109) was performed using a barcode expansion kit (Oxford Nanopore Technologies, NBD-104), and the final pooled library was sequenced on an R9.4.1 flow cell for 20 hrs.

### Data Processing of Long-Read Sequencing Results

Fast5 files were base called in real time using Miknow (v21.06.9) with the fast base calling model, and the resulting FastQ files were filtered for Q score>8. BBDuk from the BBTools suite⁶⁵ was used to filter for reads containing 20 bp of LE and RE and 30 bp of target site sequence with a maximum hamming distance of 2. Of these reads, those containing a 20 bp sequence (with a maximum hamming distance of 2) found in the plasmid backbone (not expected to occur in simple insertion products) were categorized as potential cointegrates and those not containing this sequence were categorized as potential simple insertions. Reads for plasmid-targeting experiments were additionally filtered for appropriate read length. Reads containing products assigned as simple insertions or cointegrates were merged into a single FastQ file and aligned to either a synthetic simple insertion or cointegrate product with Minimap2⁶⁶ specified with the map-ont parameter. Coverage plots were generated from an exemplary set of 100 reads using Geneious (v2021.2.2) and its inbuilt aligner (medium sensitivity and an iteration of up to 5 times). Sam files containing aligned reads were also produced and used to generate length histograms.

For sequencing results obtained from human lysate experiments, FastQ files were also filtered for Q score>8, 20 bp of LE and RE, and 30 bp of target site sequence with a maximum hamming distance of 2. Reads containing a 20 bp sequence found in the plasmid backbone were categorized as cointegrates whereas those that did not were categorized as “total”. Filtered reads were aligned to a synthetic reference using Geneious (v2021.2.2) and its inbuilt aligner (medium sensitivity and an iteration of up to 5 times) and manually inspected. Cointegrate percentage was calculated as the number of cointegrate-categorized reads divided by the number of “total”-categorized reads.

### Analysis of Insertion Distance Using Targeted Sequencing

PAM-to-LE insertion distances were assessed by next-generation sequencing using a 2-step PCR-based library construction method. 50 ng of genomic DNA from genome-targeting experiments were PCR amplified using Q5 High-fidelity DNA Polymerase (NEB) and primers which bind just outside of TS2 or just inside of LE (Supplementary Table 3). Thermal cycling conditions were: 98 for 2 min followed by 25 cycles of (98° C. for 10 sec, 64° C. for 15 sec, 72° C. for 20 sec) and a final extension of 72° C. for 3 min. PCR products were analyzed by QIAxcel capillary electrophoresis (Qiagen) and purified using paramagnetic beads prepared as previously described^{67,68}. 20 ng of purified PCR product was used as template for a second PCR to add Illumina barcodes and adapter sequences (Supplementary Table 3). Thermal cycling conditions were: 98° C. for 2 min followed by 10 cycles of (98° C. for 10 sec, 65° C. for 30 sec, 72° C. for 30 sec) and a final extension of 72° C. for 5 min. PCR products were analyzed and purified prior to quantification via QuantiFluor (Promega) and combined into an equimolar pool. Final libraries were quantified by qPCR (KAPA Library Quantification Kit; Roche 7960140001) and sequenced on a MiSeq using a 300-cycle v2 kit (Illumina).

### Data Processing of Targeted Sequencing Results

Paired FastQ reads were first filtered for Q>30 using BBDuk from the BBTools suite and merged via BBMerge. Reads containing 20 bp of TS2 and 20 bp of the terminal LE, each with a maximum hamming distance of 1, were then extracted. Each read was then trimmed of the sequence upstream of and including the PAM and downstream of and including the LE, resulting in only the sequence between the PAM and LE (i.e. site of insertion). Lengths of the resulting reads were calculated and used to plot PAM-to-LE insertion distance profiles.

### Unbiased, Genome-Wide Specificity Analyses

Two versions of specificity analysis library preparation were carried out depending on donor plasmid origin (R6K or SC101). When using R6K origin donors, transposition experiments were carried out by heat shocking 25 ng each of pDonor and pCAST or pHELIX into PIR2 cells. After 18 hours of growth on agar plates containing 50 μg/mL Kanamycin and 25 μg/mL Carbenicillin, colonies were scraped and gDNA extracted using Wizard Genomic Purification Kit (Promega).

When using temperature sensitive SC101 origin donors, electroporations with 100 ng each of pDonor and pCAST or pHELIX were performed using electrocompetent Endura cells. Cells were recovered in S.O.C at 30° C. for 1 hour before 100 μL of recovery was inoculated into 3 mL of LB media containing Kanamycin and Carbenicillin. Cultures were shaken at 750 RPM at 30° C. for 8 hours. 150 μL of culture was plated on Carbenicillin containing agar plates and grown for 14 hours at 42° C. Resulting colonies were scraped and gDNA extracted using Wizard Genomic Purification Kit (Promega), with a final resuspension step done in Buffer EB (Qiagen), which does not contain EDTA.

600 ng of gDNA was used as input into library preparation using HyperPlus Kit (Roche). Briefly, gDNA was subject to enzymatic random fragmentation for 8 min, ligations were performed with the fragmented gDNA, and Stubby Adaptors (IDT) for 90 min, and adaptor-ligated fragments were bead cleaned using 0.9× Ampure XP beads (Beckman Coulter) (all according to the manufacturers protocol). If R6K origin donors were utilized, adaptor ligated fragments were subject to double digestion by NruI and ScaI for 6 hours at 37° C. to deplete fragments resulting from uninserted donor (for SC101 origins, uninserted donor was heat cured in the previous step) and bead cleaned with 0.9× Ampure XP beads. Next, genome-LE junctions were enriched via a PCR with Q5 High-fidelity DNA Polymerase (NEB) using an i7-specific primer and a transposon LE specific primer containing an i5 adaptor sequence (Supplementary Table 3). Thermal cycling conditions were: 98 for 2 min followed by 25 cycles of (98° C. for 10 sec, 66° C. for 15 sec, 72° C. for 30 sec) and a final extension of 72° C. for 2 min. 50 ng of purified PCR product was used as template for a second, 10-cycle PCR to add Illumina barcodes and adapter sequences (Supplementary Table 3). Final libraries were quantified by Quibit Fluorimeter and submitted to the Walk-Up Sequencing service at the Broad Institute of MIT and Harvard for sequencing on a high-output 75-cycle NextSeq sequencing kit.

### Data Processing of Specificity Analysis Results

Single end, adaptor trimmed, and demultiplexed reads from specificity analysis NGS were filtered for Q>20 and used for downstream processing using BBDuk from the BBTools suite. Reads containing 20 bp of ShCAST LE were extracted, and the resulting reads containing 20 bp of the donor backbone were removed. Remaining reads contained the genome-LE junction. Next, reads were trimmed of the LE sequence, leaving only the LE-adjacent genome sequence, and mapped to the E. coli genome (GenBank: U00096.2). Mapped reads were filtered for those that aligned uniquely. Coordinates of uniquely aligned reads were used for specificity calculations and visualization, where an on-target insertion event was defined as one that occurred within 55-75 bp downstream of the PAM.

### Human Cell Culture

Human HEK 293T cells (ATCC) were cultured at 37° C. with 5% CO₂ in Dulbecco's Modified Eagle Medium (DMEM) supplemented with 10% heat-inactivated FBS and 1% penicillin/streptomycin (ThermoFisher). The supernatant media from cell cultures was analyzed monthly for the presence of mycoplasma using MycoAlert PLUS (Lonza).

### Transposition Assays Targeting Plasmids in Human Cell Lysates

Approximately 150,000 HEK 293T cells per well were seeded in 24-well plates ˜20 hours prior to transfection. Transfections were performed using 600 ng of DNA and 1.8 μL of TransIT-X2 (Mirus), whether using a single all-in-one plasmid or when components were expressed from individual plasmids (for the latter, 150 ng of each plasmid encoding NLS-Cas12k, NLS-TniQ, TnsC, NLS-nAniI-TnsB or NLS-TnsB was used). Transfected cells were incubated for 48 hrs at 37° C., and then the cell lysate was harvested by removing culture medium and adding 100 μL of lysis buffer (20 mM Hepes pH7.5, 100 mM KCl, 5 mM MgCl₂, 5% (vol/vol) glycerol, 1 mM DTT, 0.1% (vol/vol) Triton X-100, and 1× SigmaFast Protease Inhibitor Cocktail (EDTA-free) (where 1× solution is 1 tablet per 100 mL)) to each well and placed on a rocker for 20 min at 4° C. Suspended cells were placed in a 96-well PCR plate, vortexted vigorously for 3-5 sec, and briefly spun down in a centrifuge to remove cell debris. Lysates were then aliquoted into PCR-strip tubes and snap frozen via liquid nitrogen for further use.

N₇CAST sgRNAs were in vitro transcribed (T7 RiboMax Express Large Scale RNA Production System; Promega) using PCR templates that added a T7 promoter and the TS1 spacer to the sgRNA scaffold (Supplementary Table 3). For transposition reactions, 15 μL of cell lysate was combined with 20 ng pTarget, 100 ng N₇HELIX pDonor, and 1 mg TS1-targeting sgRNA. Reactions were gently mixed and incubated at 37° C. for 4 hrs. To stop the reaction, 0.8 U Proteinase K (NEB) was added to each reaction, and reactions were incubated at room temperature for 15 min before a heat inactivation step of 95° C. for 10 min. 2 mL of the terminated and heat-inactivated product was used as input for junction PCRs and long-read sequencing enrichment (as described above).

### Transposition Assays Targeting Plasmids in Human Cells

Approximately 20,000 HEK 293T cells were seeded in 96-well plates ˜20 hours prior to transfection.

Transfections were performed using 0.6 μL of TransIT-X2 (Mirus) with 0.5, 1, 2, or 10 ng pTarget, 80 ng of all-in-one N₇CAST or N₇HELIX plasmid, 60 ng of N₇HELIX pDonor, 20 ng of CMV-sgRNA1 or U6-sgRNA2 plasmid, and if applicable, 20 ng of HU expression plasmid and/or 20 ng of N₇S15 expression plasmid. Transfected cells were incubated at 37° C. for 72 hours, culture media was removed, and cells were lysed by addition of 100 μL of lysis buffer (20 mM Hepes pH7.5, 100 mM KCl, 5 mM MgCl₂, 5% (vol/vol) glycerol, 1 mM DTT, 0.1% (vol/vol) Triton X-100). The lysis reaction was and incubated at 65° C. for 6 min followed by 98° C. for 2 min. DNA (gDNA/plasmid mixture) was extracted by performing a clean-up reaction on the lysate using 1× Ampure XP beads, then used as input into junction PCRs and ddPCR (as described above).

### Example 1. Development and Optimization of HELIX

We first sought to engineer a cointegrateless type V-K CAST capable of cut-and-paste transposition by restoring the absent function of TnsA. To do so, we initially created fusions of TnsA enzymes (from various Tn7 transposons or ones that occur as natural TnsA-B fusions in type I CASTs) to TnsB of the canonical type V-K CAST from Scytonema hofmannii (ShCAST). The N-terminal domain of E. coli Tn7 TnsA carries out 5′ donor cleavage whereas the C-terminal domain interacts with downstream transposition components^{33,24}. Predicted structures of additional TnsA enzymes that we sought to examine also revealed distinction between the N- and C-terminal domains (FIG. 6a). Since the C-terminal domain of TnsA would not be predicted to play a functional role in transposition when combined with an orthogonal type V-K CAST, we chose to fuse N-terminal domains of various TnsAs to ShTnsB. Assessment of ShCAST integration with the TnsA-TnsB fusions revealed a substantial reduction in integration efficiency compared to wild-type ShCAST (FIG. 6b). Furthermore, for the three TnsA-TnsB fusions that exhibited detectable integration, we observed only in one case a moderate decrease in the insertion product cointegrate fraction (FIG. 6c) while also observing an increased proportion of insertions occurring into the pEffector plasmid (FIG. 6d).

Next, we considered the use of LAGLIDADG HE (LHE) fusions to TnsB. LHEs have been harnessed for genome editing in bacterial and human cells and have moderate reprogrammability via protein engineering or chimeric assembly³⁴. The LHE from Aspergillus nidulans (I-AniI) has a small coding sequence (254 amino acids), cleaves a 19-bp asymmetric DNA target sequence, and has been previously engineered to be a sequence-specific nickase through a single K227M mutation²⁹ (nAniI). Furthermore, a hyperactive variant of I-AniI, termed Y2 I-AniI, has been shown to have a 9-fold higher affinity for its cognate target site³⁵. We hypothesized that fusion of either nAniI or Y2 nAniI to TnsB (creating HELIX fusion proteins) could enable dual nicking on the donor plasmid required for cut-and-paste DNA insertions with type V-K CASTs (FIG. 1c). Importantly, recognition sequences for nAniI could be encoded on the donor plasmid backbone without complicating or restricting RNA-programmed targeting. Furthermore, the length of the nAniI recognition sequence makes undesired nAniI-mediated nicking at the Cas12k-bound target site, due to TnsB-localization, unlikely.

We therefore determined whether nAniI could adequately substitute for the lack of TnsA in ShCAST. To do so, we constructed a series of ShCAST expression plasmids that each contained: (1) a single guide RNA (sgRNA) targeting target site 1 (TS1) on a separate target plasmid (pTarget), (2) Cas12k, (3) TniQ, (4) TnsC, and (5) nAniI fused to the N- or C-terminus of TnsB (FIG. 1d). ShCAST expression plasmids were co-transformed with a previously described donor plasmid (pDonor)¹⁴ (containing a 2.1kb cargo and ShCAST left and right transposon ends (LE and RE, respectively)), into an E. coli strain harboring pTarget (FIG. 1d). To determine whether ShCAST retained transposition activity with TnsB fusions to nAniI, we assessed integration by performing junction PCR across both the LE and RE within pTarget on miniprepped DNA from pooled colonies harboring transposed products. Fusion of nAniI to the N-terminus of TnsB supported RNA-guided DNA insertion while C-terminal fusions did not (FIG. 1e), suggesting that the C-terminal TnsC interacting domain of TnsB is less accommodating to fusion proteins³⁶. Recent structural studies of ShCAST TnsB support this finding due to the observation that a 15 residue C-terminal “hook” in TnsB is the primary means of physical TnsB-TnsC association^{37,38}. Henceforth, the nAniI-TnsB fusion architecture along with the remaining CAST components is referred to as HELIX (FIG. 1c).

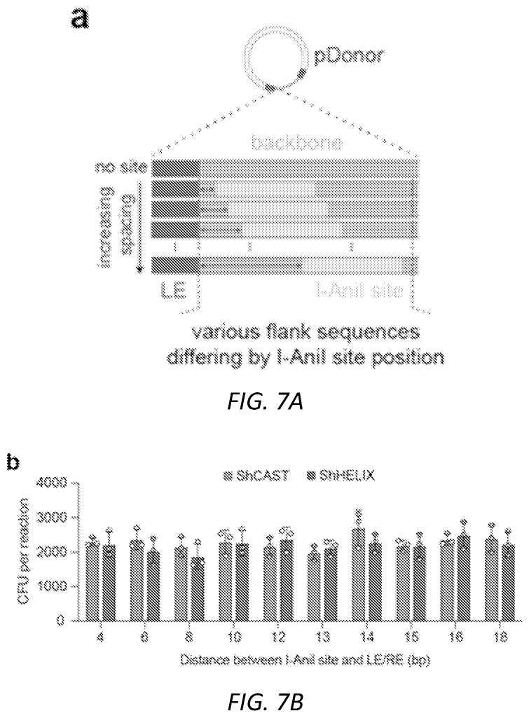

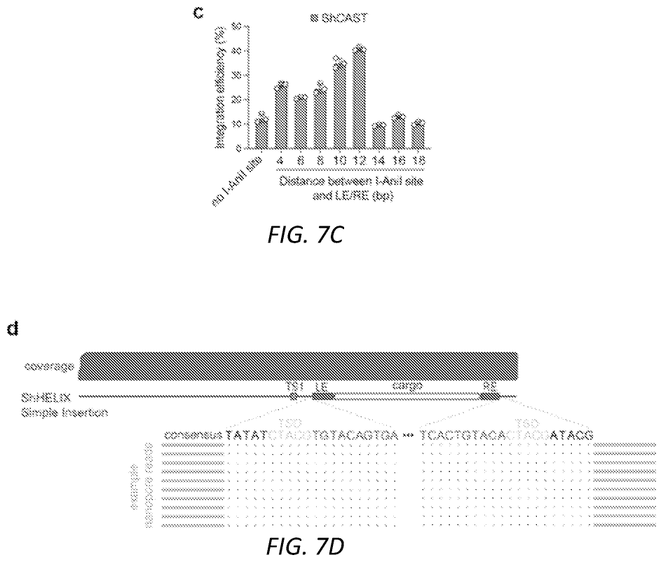

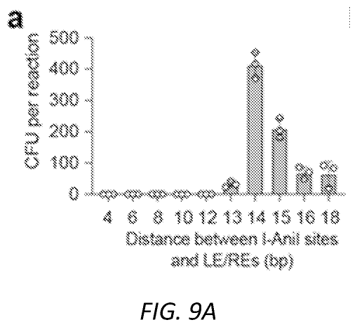

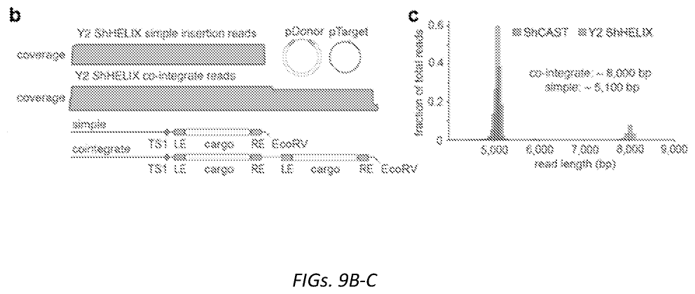

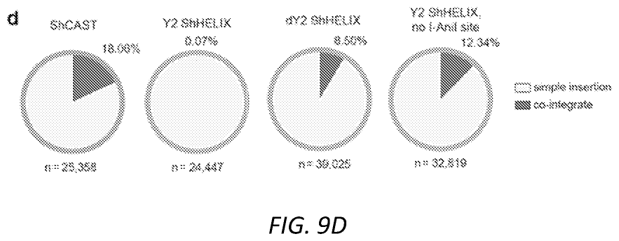

Next, to generate the 5′ nick on pDonor via nAniI, we encoded the I-AniI target sequence on a series of donor plasmids with variable distances to the LE/RE (FIG. if and FIG. 7a). When co-transforming ShCAST or ShHELIX plasmids along with various pDonors into our pTarget strain, we observed similar numbers of transformant colonies, suggesting comparable cell-viability (FIG. 7b). With ShHELIX, we observed a range of integration efficiencies, assessed via droplet digital PCR (ddPCR), across different I-AniI-LE/RE spacings on pDonor, with a 14 bp spacing yielding the highest integration (FIG. 1f). Surprisingly, ShCAST also exhibited variable integration efficiency depending on the spacing between the I-AniI site and LE/RE (where, unlike with ShHELIX, the I-AniI site has no direct role in transposition). For ShCAST, pDonors with spacings of 4-12 bp resulted in substantially higher insertion efficiencies than a pDonor without I-AniI sites (FIG. 7c). Altering the position of the I-AniI site modifies the sequence directly adjacent to the LE/RE on pDonor, suggesting that the composition of the flanking sequence, particularly the first 12 bp, may be an important determinant of integration efficiency (FIGS. 7a and 7c). Separately, we also performed integration experiments using Y2 nAniI fused to TnsB (Y2 ShHELIX) and observed substantially fewer colonies, with peak numbers using 14 bp spacing (FIG. 9a and Example 7). For subsequent experiments, HELIX constructs with nAniI-TnsB fusions and pDonors with 14 bp between the I-AniI sites and LE/RE were used.

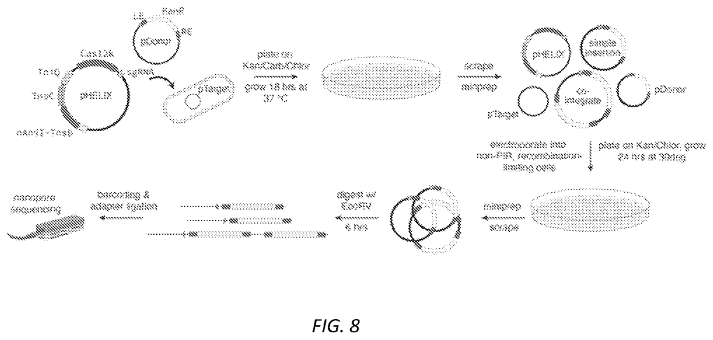

Next, we employed long-read sequencing to assess whether restoration of the 5′ nick on pDonor with ShHELIX could improve product purity compared to ShCAST. We enriched for transposed products from our miniprepped plasmid pool by retransforming into non-pir cells (eliminating uninserted donor plasmid) and selecting for insertion products (FIG. 8), linearized extracted plasmid DNA, and performed long-read sequencing to determine the proportion of simple insertions to cointegrates (FIGS. 1g-1i). With ShCAST, we observed 18.06% cointegrates, consistent with previous results⁶ (FIG. 1i). Strikingly, ShHELIX nearly eliminated cointegrates, resulting in a reduction to only 0.49% of all products (a 37-fold decrease when compared to ShCAST; FIGS. 1h and 1i). Expression of unfused nAniI along with ShCAST did not lead to a reduction in cointegrates, demonstrating that fusing nAniI to TnsB is critical to HELIX function (FIG. 1i). Additionally, we did not observe I-AniI sites in insertion product reads, suggesting that the 5′ flap harboring these sequences are removed during HELIX-mediated transposition (FIG. 1c and FIG. 7d). We also performed long-read sequencing of Y2 ShHELIX products and similarly observed an improvement in simple insertion product purity only with Y2-nAniI (FIGS. 9b-d).

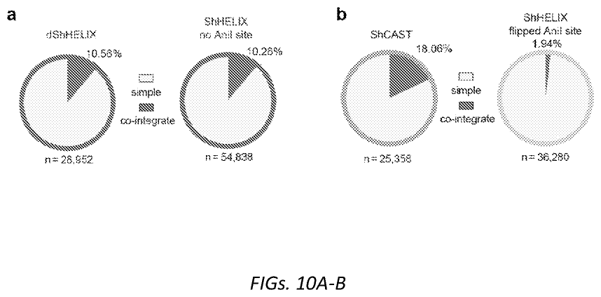

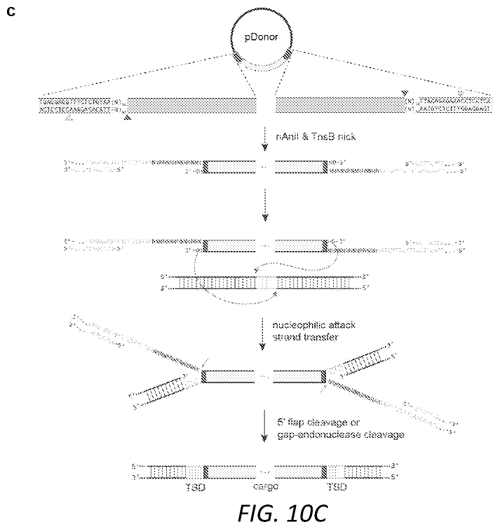

We also performed a series of control experiments to further characterize ShHELIX (Example 8). First, a catalytically attenuated variant of I-AniI (K227M, Q171K) decreased cointegrates 1.7-fold compared to ShCAST (presumably due to incomplete inactivation of I-AniI nicking) (FIG. 10a). Secondly, a pDonor lacking an I-AniI target site resulted in a 1.7-fold reduction in cointegrates compared to ShCAST (FIG. 10a and Example 8). Next, experiments using a pDonor with a “flipped” I-AniI site that places the nick on the same strand as the TnsB nick resulted in a 9-fold decrease in cointegrates (FIG. 10b). The resulting “gapped” Shapiro intermediate may be processed by 5′ flap endonuclease and/or gap endonucleases³⁹ (in addition to the possibility of low-level DSB-mediated cargo excision) to result in simple insertion products (FIG. 10c). Finally, when a “Lib4” variant target site for I-AniI (found previously to increase the affinity of wild type I-AniI by 5-fold⁴⁰) was used on pDonor, we observed a further reduction of cointegrates to 0.18% of all transposition products (for a 100-fold decrease in cointegrates compared to ShCAST) (FIG. 1j). However, this product purity improvement was also accompanied by a reduction in CFUs (Example 7 and FIG. 1k) so was not used in further experiments. Altogether, ShHELIX coupled with an I-AniI site oriented on pDonor to confer a 5′ nick demonstrated the most prominent increase in simple insertion to cointegrate percentage, leading to near-perfect product purity on a plasmid target.

### Example 2. Characterization of HELIX on Genomic Targets

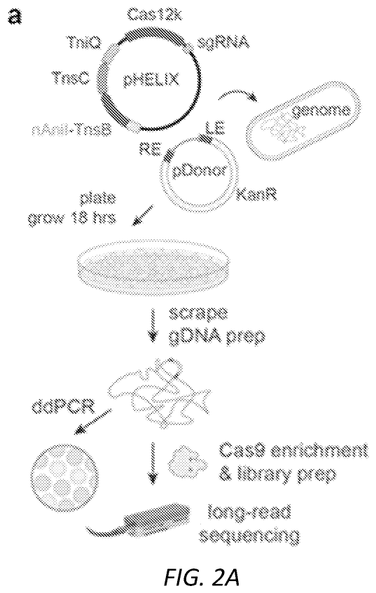

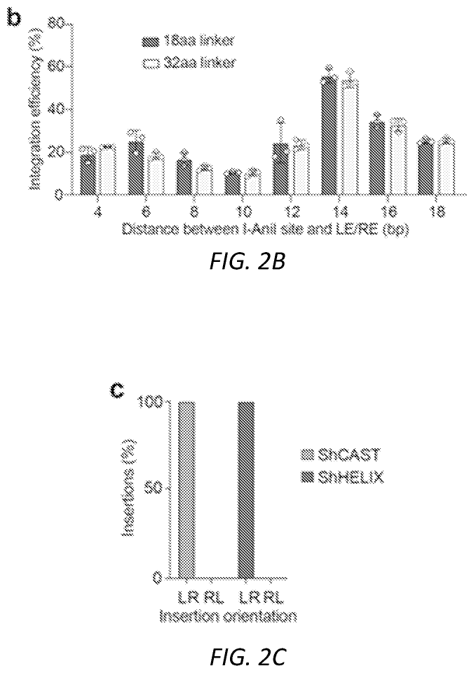

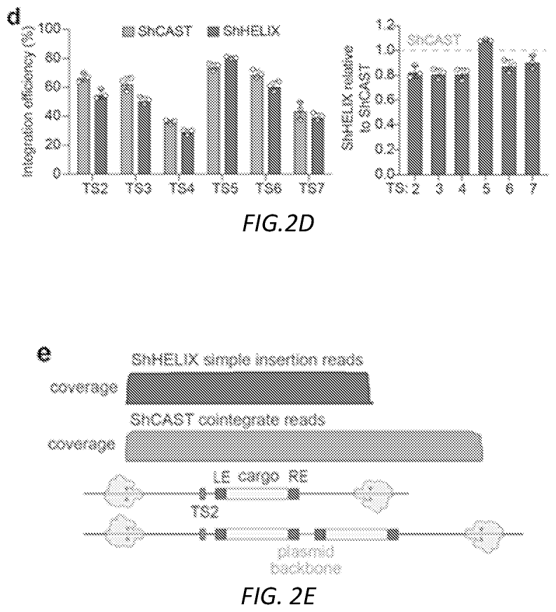

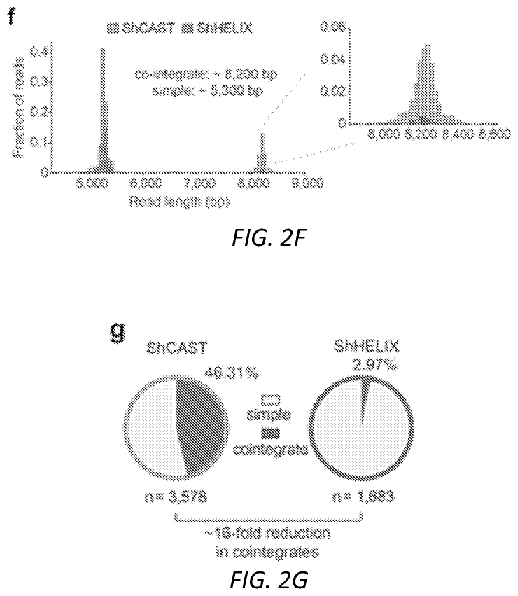

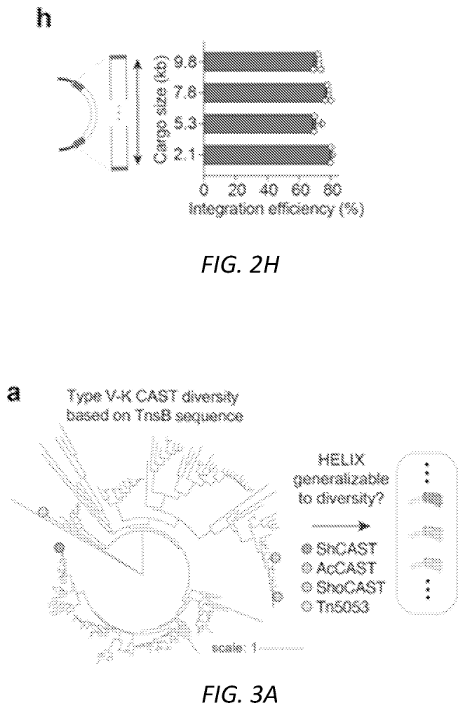

Encouraged by our transposition results on plasmid targets, we then explored the efficacy of ShHELIX-mediated DNA integration at genomic sites. We performed transformations using similar constructs to the plasmid targeting experiments but instead with genome-targeting sgRNAs and without pTarget (FIG. 2a). First, we tested the effect of two different lengths of amino acid linkers between nAniI and TnsB on genomic integration efficiency across our set of eight donor plasmids containing varying distances between the I-AniI sites and the LE/RE. Experiments were performed with a previously characterized sgRNA¹⁴ against a genomic target site (TS2). For both amino acid linkers, we observed the highest integration efficiency with a 14 bp spacing between the I-AniI site and LE/RE (FIG. 2b), which aligned with our plasmid targeting results. All detectable insertions were in the T-LR orientation (FIG. 2c).

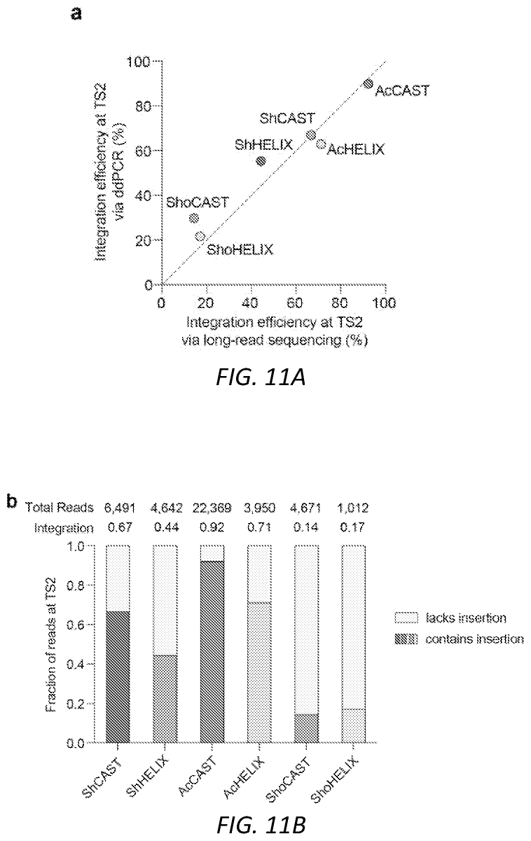

Having identified an optimal I-AniI site to LE/RE spacing on pDonor for genome targeting, we then compared the integration efficiencies and product purities of ShCAST and ShHELIX across a range of genomic sites. ShHELIX retained robust RNA-programmed integration across six genomic target sites at levels comparable to ShCAST (FIG. 2d). To analyze the on-target product purity of HELIX integrations when targeting the genome at TS2, we utilized long-read sequencing (following an in vitro Cas9-based genomic target enrichment strategy⁴¹). Analysis of target-enriched reads when using ShCAST and ShHELIX that contained or lacked the cargo insertion showed that integration efficiencies calculated from our long-read sequencing data were similar to our ddPCR results at TS2 (FIG. 11a). With ShCAST, we observed that 46.31% of insertion reads were cointegrates (FIGS. 2e-g), which is generally lower than previously observed, albeit against a different target site and via alternate long-read sequencing methods¹⁷. With ShHELIX, we observed only 2.97% cointegrates, a 16-fold decrease compared to ShCAST (FIGS. 2e-g).

Next, we assessed the ability of ShHELIX to integrate DNA cargos of various sizes. We performed transposition experiments using donor plasmids harboring cargos of either a 5.2, 7.8, or 9.8 kb sequence (compared to pDonor with a 2.1 kb cargo used in previous experiments). When transposing each cargo, ShHELIX showed comparably high efficiency of targeted DNA integration irrespective of cargo size (FIG. 2h). Together, our results demonstrate that ShHELIX is capable of highly active, unidirectional, cut-and-paste DNA insertions and is insensitive to cargo sizes up to at least 10 kb.

### Example 3. Extensibility of HELIX to Type V-K CAST Orthologs

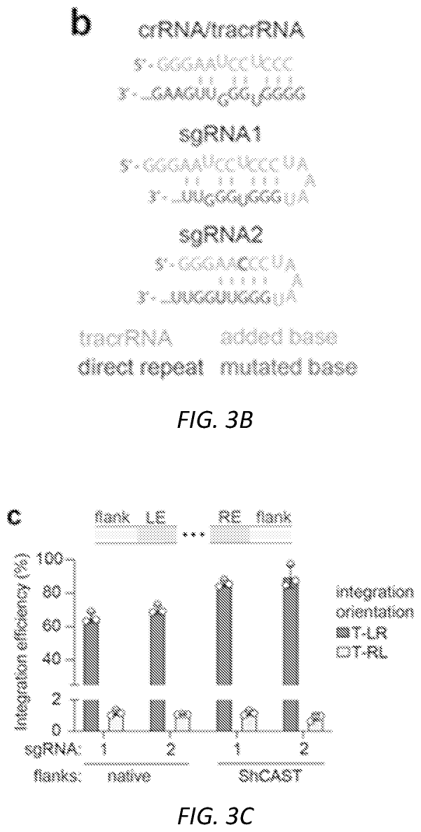

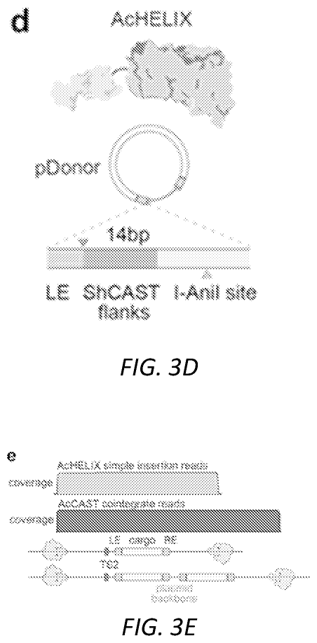

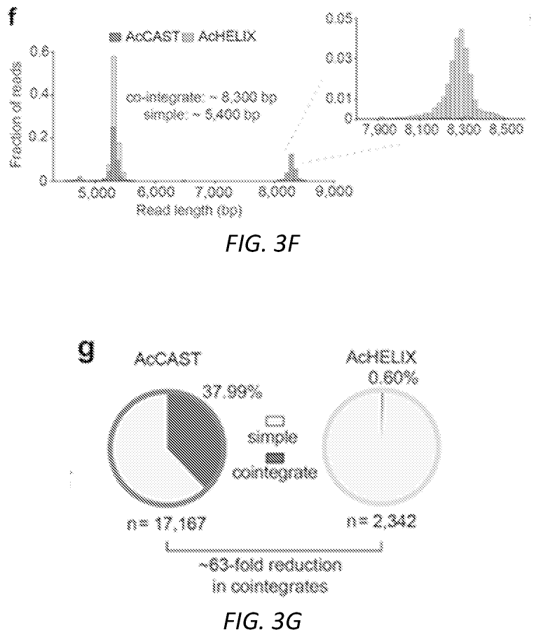

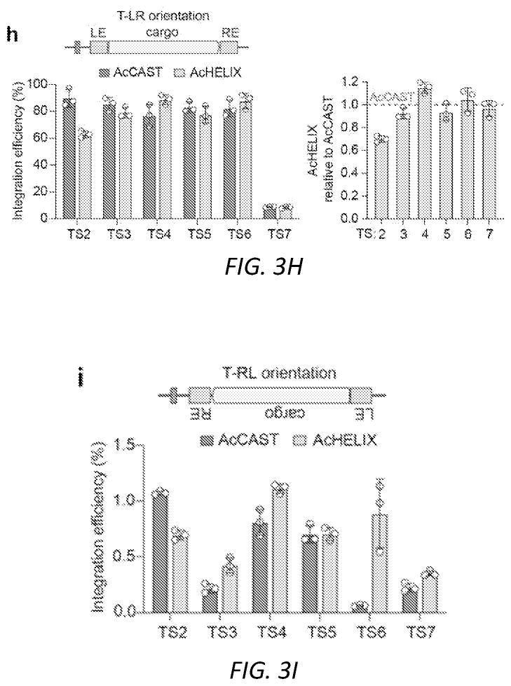

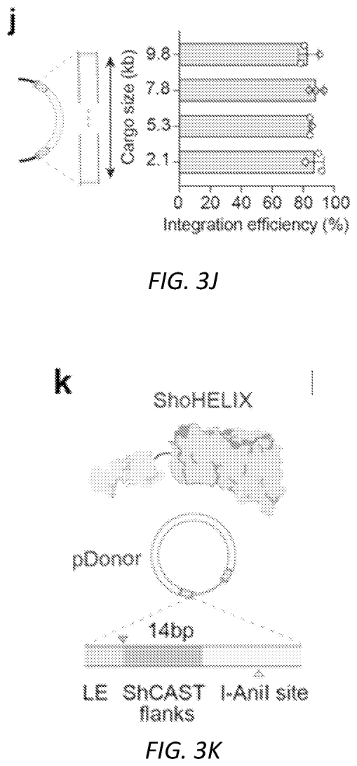

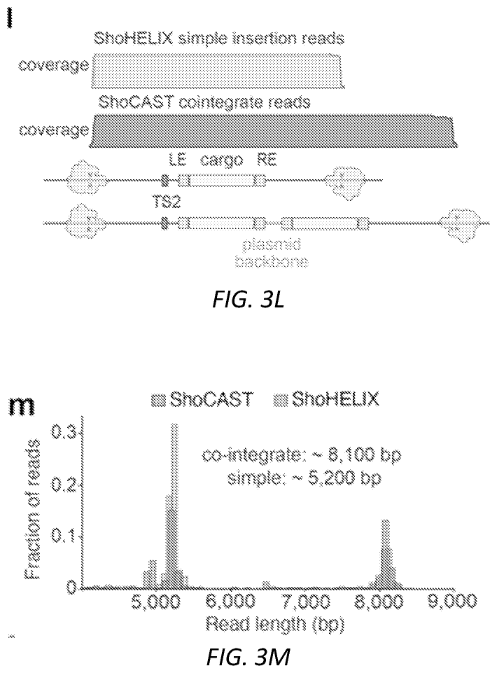

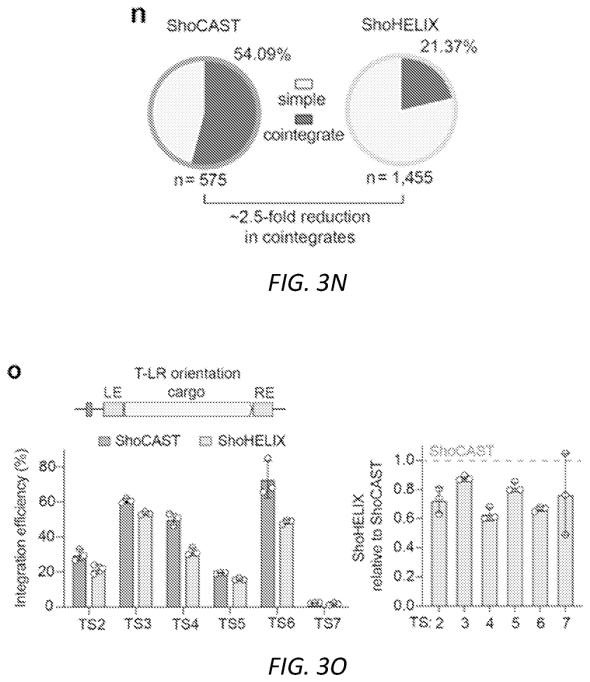

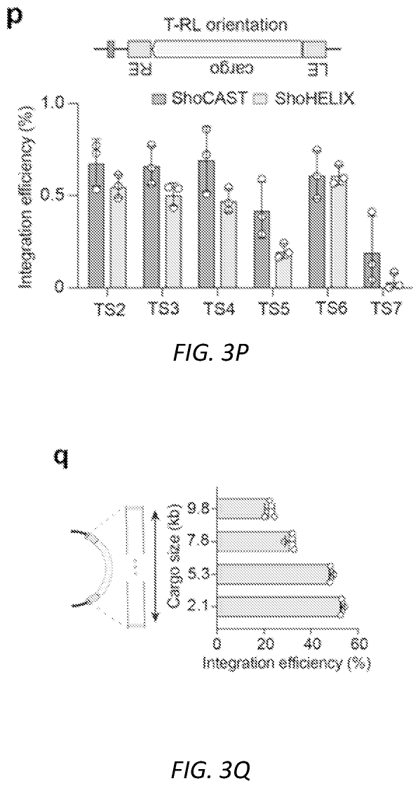

All discovered type V-K CASTs lack TnsA²¹. This observation supports an evolutionary hypothesis that a Tn5053-like transposon, containing TnsB, TnsC, and TniQ, but not TnsA, co-opted and repurposed this CRISPR system. Therefore, all type V-K CASTs would be expected to act through replicative transposition, leading to a substantial fraction of undesired cointegrate products. Thus, we explored HELIX as a generalizable approach to enable cut-and-paste DNA insertion with other diverse type V-K CASTs (FIG. 3a).

To investigate the applicability of HELIX to other CAST orthologs, we characterized and optimized two previously reported type V-K CASTs from either Anabaena cylindrica (AcCAST) or a different strain of Scytonema hofmannii (ShoCAST). First, for the canonical AcCAST system, we designed two sgRNA scaffolds (FIG. 3b) and two pDonor architectures, the latter of which varied by containing different 25 bp sequences flanking the LE and RE (either as previously reported for AcCAST¹⁴ or using the ShCAST flanking sequences). With the two sgRNA designs that differed based on their crRNA-tracrRNA fusion points, we observed only a modest difference in integration efficiency (FIGS. 3b and 3c). However, the pDonor containing ShCAST flanking sequences resulted in increased absolute integration efficiencies of 19.6% or 20.4% for sgRNA1 and sgRNA2, respectively (1.28- and 1.31-fold increases over pDonor with the native AcCAST flanks; FIG. 3c). As we previously observed for ShCAST (FIG. 7c), these results suggest that the sequences directly adjacent to the LE and RE on pDonor are an important determinant of type V-K CAST-mediated integration efficiency. Additionally, AcCAST showed a minimal, though still detectable, number of T-RL oriented insertions, making it a near-complete unidirectional inserter (FIG. 3b).

We constructed AcHELIX comprising a nAniI-TnsB fusion along with the sgRNA2 design and a pDonor harboring I-AniI sites 14 bp from the LE/RE separated by ShCAST flanking sequence (FIG. 3d). To determine the integration product purity with AcHELIX compared to AcCAST when targeting the genome, we performed long-read sequencing following Cas9 target enrichment (FIG. 3e). While with AcCAST we observed 37.99% cointegrate products, for AcHELIX we found only 0.60%, representing a 63-fold improvement in product purity with AcHELIX (FIGS. 3f and 3g). Across six genomic targets, AcHELIX retained comparable RNA-guided DNA integration and insertion directionality to AcCAST (FIGS. 3h, 3i and FIGS. 11a and 11b). Additionally, similar to ShHELIX, AcHELIX demonstrated no decrement in efficiency when integrating cargo sequences of various sizes up to 9.8 kb, maintaining over 83% integration efficiency for all four cargo sizes at TS6 (FIG. 3j). Thus, similar to ShHELIX, AcHELIX is an efficacious engineered CAST with near-perfect simple insertion product purity for DNA insertions of various sizes.

Next, we characterized ShoCAST and ShoHELIX utilizing a pDonor with a 14 bp spacing separating the I-AniI site and LE/RE with ShCAST flanking sequence (FIG. 3k). We performed genome-targeting experiments with ShoCAST and ShoHELIX using a previously reported sgRNA¹⁶ against TS2. Characterization of the insertion products via long-read sequencing revealed 54.09% cointegrates for ShoCAST and 21.37% for ShoHELIX, demonstrating a 2.5-fold reduction in cointegrates when using ShoHELIX (FIGS. 3l-3m). Across genomic targets TS2-TS7, we observed a range of integration efficiencies, with ShoHELIX exhibiting comparable integration to ShoCAST (FIG. 3o and FIGS. 11a and 11b). Similar to AcCAST and AcHELIX, the directionality of ShoCAST and ShoHELIX insertions were predominantly in the T-LR orientation, albeit with detectable T-RL insertions (FIG. 3o and 3p). Additionally, in contrast to ShHELIX and AcHELIX, ShoHELIX showed a decrease in integration efficiency with increasing cargo size on pDonor at TS3 (FIG. 3q). Finally, to test whether nAniI fusion to TnsB altered the distance between the PAM and insertion site, we conducted amplicon sequencing across genome-LE junctions (FIG. 12a). ShHELIX, AcHELIX, and ShoHELIX did not alter the insertion distance profiles of their canonical CAST (FIG. 12b-7g).

### Example 4. Comparison of Type I, Type V-K, and HELIX Systems

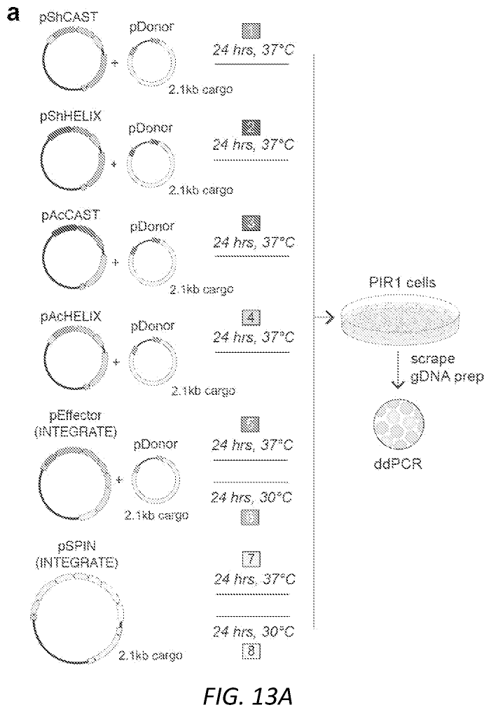

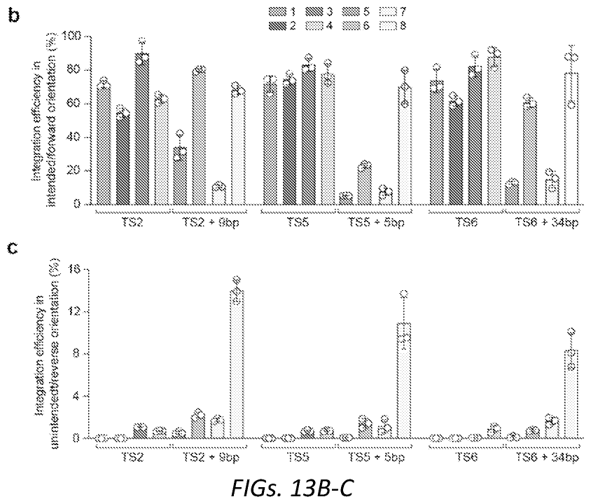

Since a streamlined type I CAST, termed INTEGRATE, was recently described¹⁶, we sought to compare the efficiency and directionality of integration with ShHELIX and AcHELIX with Vibrio Cholerae INTEGRATE. We conducted transposition assays which controlled for growth time (24 hrs), donor cargo size (2.1kb), approximate donor copy number (high copy), cell type (PIR1), general genomic target location (according to closest compatible PAMs), and efficiency measurement method (ddPCR) (FIG. 13a). We found that HELIX is more efficient or comparably efficient to INTEGRATE depending on constructs used and growth temperature (FIG. 13b). Notably, for INTEGRATE-mediated insertions performed at 30° C., we observed substantial integration in the reverse orientation (FIG. 13c).

### Example 5. Characterization and Optimization of Type V-K CAST and HELIX Specificity

In contrast to the high-specificity insertion profiles of type I CASTs, type V-K CASTs are prone to off-target integration spread across the bacterial genome^{14,16,17,20}. Recent structural studies of ShCAST have revealed Cas12k-independent TnsC filamentation on DNA in a sequence-agnostic manner^{36,42,43} (similar to MuB in Mu transposase⁴⁴), potentially leading to off-target integration due to untargeted assembly of the transpososome. TniQ has also been shown to play a crucial role in transposition events by capping and nucleating TnsC filaments^{42,43}. Therefore, one potential approach to increase the specificity of type V-K CASTs would be to fuse TnsC and/or TniQ to Cas12k to localize transposition events to Cas12k-target-bound DNA.

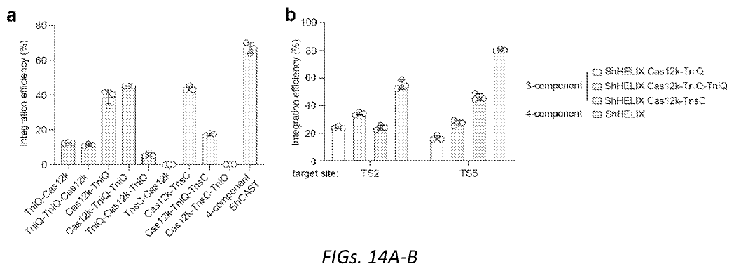

To test this hypothesis, we constructed various 3-component ShCAST systems where Cas12k was fused with TniQ or TnsC in every orientation, as well as two component systems with Cas12k, TniQ, and TnsC fused (FIG. 4a). Transposition experiments demonstrated that Cas12k-TniQ, Cas12k-TniQ-TniQ, and Cas12k-TnsC fusions retained a majority of their activities relative to unfused canonical CAST (FIG. 4b and FIG. 14a). HELIX versions of these three best performing fusion constructs also maintained appreciable integration at TS2 and TS5 (FIGS. 4c, 4d and FIG. 14b). Furthermore, ShCAST and ShHELIX with Cas12k fusions did not alter the distance between the PAM and the integration site (FIG. 12h-7m). Both ShCAST and ShHELIX with or without Cas12k-TnsC fusions preserved target immunity (FIG. 4e), whereby sites that have undergone integration events become resistant to subsequent integrations^{14,45,46}. Our observations that Cas12k-TniQ fusions retain functionality, combined with identical insertion distance profiles for all fusions, supports proposed models where Cas12k and TniQ are directly associated during transposition^{42,43}.

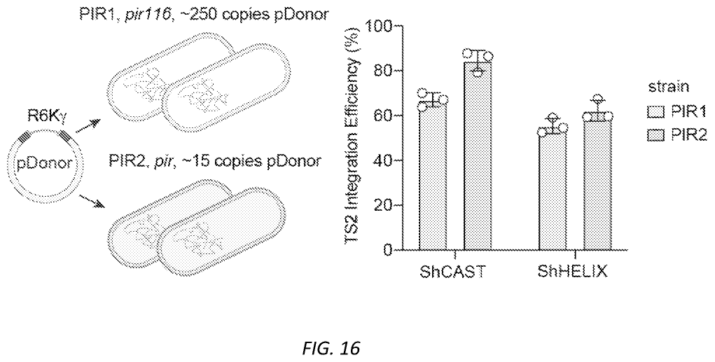

To compare the specificities of ShCAST, ShHELIX, and versions with Cas12k-TniQ or -TnsC fusions, we conducted an unbiased analysis of genome-wide integration. Similar to previously described methods^{14,16,20}, we performed transformations in Endura cells and analyzed insertion specificity via random enzymatic fragmentation of genomic DNA followed by integration junction enrichment and sequencing. Our results revealed 54.4% on-target integration when targeting TS2 with ShCAST (FIG. 4f), a specificity profile that aligns with previously reported values for this target site¹⁴. Strikingly, ShHELIX exhibited 88.4% on-target integration with the TS2 sgRNA, a 34% absolute increase in on-target specificity compared to ShCAST (FIG. 4f and FIGS. 15a, 15b). Moreover, using ShHELIX with a donor not containing I-AniI sites or dShHELIX (containing a catalytically dead I-AniI) also demonstrated >88% on-target specificity (FIG. 15b), indicating that neither I-AniI binding nor cleavage is the primary cause of this 1.6-fold enhanced specificity. Instead, these results potentially indicate that fusion of nAniI to TnsB structurally alters CAST conformation and/or how TnsB distorts donor topology to energetically disfavor transposition at sites not bound by Cas12k. Analogous experiments with ShHELIX containing Cas12k-TniQ and Cas12k-TnsC fusions further improved specificity to 94.5% and 96.5% on-target integration, respectively (FIG. 4f). Comparable ShCAST specificities with Cas12k-TniQ and Cas12k-TnsC fusions were 65.3% and 51.7%, respectively (FIG. 4f and FIG. 15a). We also assessed integration specificity in another E. coli strain by conducting genome-wide insertion analyses in PIR2 cells (FIGS. 15c and 15d). Curiously, we observed enhanced on-target specificity for all conditions, with ShHELIX constructs achieving on-target integration above 97% (FIG. 4f and FIG. 15c). Furthermore, this high specificity ShCAST- and ShHELIX-mediated transposition in PIR2 cells did not decrease transposition efficiency (FIG. 16).

A major genotypic difference between Endura and PIR2 strains is the pir gene in PIR cells, which encodes the pi protein needed for conditional replication of R6K origin plasmids^{47,48}. We therefore sought to determine whether pi coexpression could increase the specificity of HELIX in non-pir cells, potentially obviating the need for efficiency-altering Cas12k fusions. To do so, we cloned separate plasmid harboring the wild-type pir gene or the pir116 mutant (shown to initiate higher copy replication of R6K origin plasmids⁴⁸), and cotransformed Endura cells with pDonor and ShCAST or ShHELIX plasmids containing a TS2 genome targeting sgRNA (FIG. 4g). Specificity profiling revealed that wild-type pi together with ShHELIX resulted in an additional absolute 7.6% boost in specificity, with 96.0% of reads occurring at the on-target site (FIG. 4h) (comparable to the specificity observed with ShHELIX and the Cas12k-TniQ or Cas12k-TnsC fusion in PIR2 cells; FIG. 4f). Coexpression of pi with ShCAST, or coexpression of mutant pi with either ShCAST or ShHELIX, led only to minor changes in specificity (FIG. 4h)

Comparative mapping of the genome-wide integration sites of ShCAST (FIG. 4i), ShHELIX with Cas12k-TniQ (FIG. 4j), ShHELIX with Cas12k-TnsC (FIG. 4k), and ShHELIX (no fusion) with pi coexpression (FIG. 4l) from specificity experiments conducted in Endura cells visualized a striking reduction in genome-wide off-target integration events when using ShHELIX systems. Moreover, comparison of specificity profiles for ShCAST with or without pi protein coexpression reveals that pi protein generally decreases the distribution of off-target integration but increases occurrence at a selection of sites (FIG. 15a). A similar trend was observed with ShHELIX and pi protein coexpression, though less drastic due to higher on-target integration specificity (FIG. 15b). Together, ShHELIX coupled with component fusions (though at the expense of some integration efficiency) as well as pi coexpression, can substantially improve the genome-wide specificity of type V-K systems, achieving levels of on-target integration comparable to type I systems^{15-17,49} while employing fewer molecular components and a smaller coding size (FIG. 17).

### Example 6. HELIX-Mediated DNA Integration in Human Cell Contexts

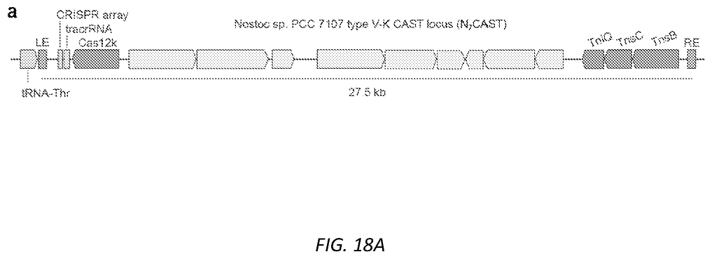

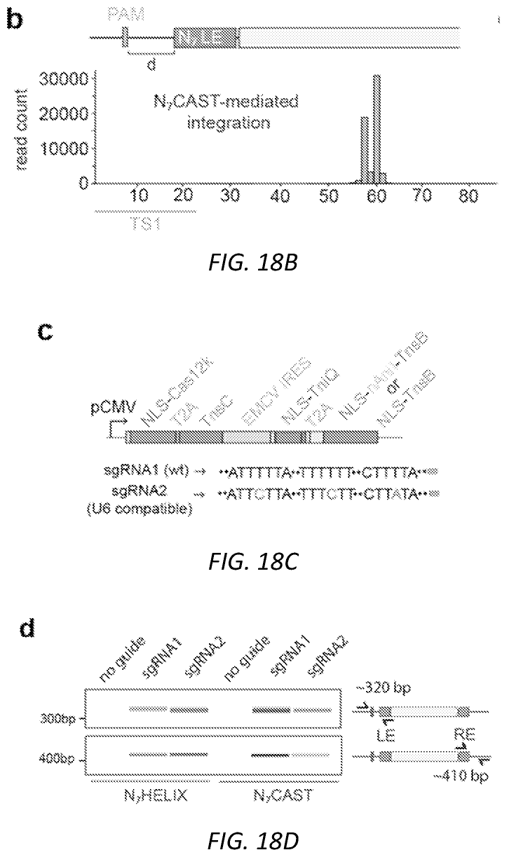

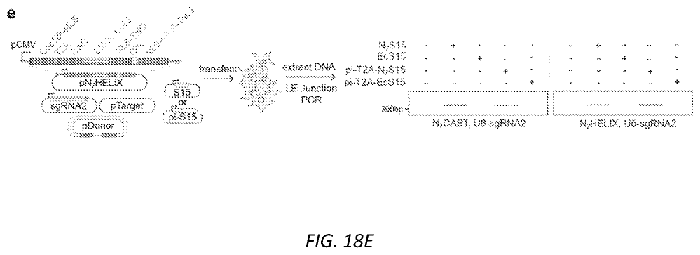

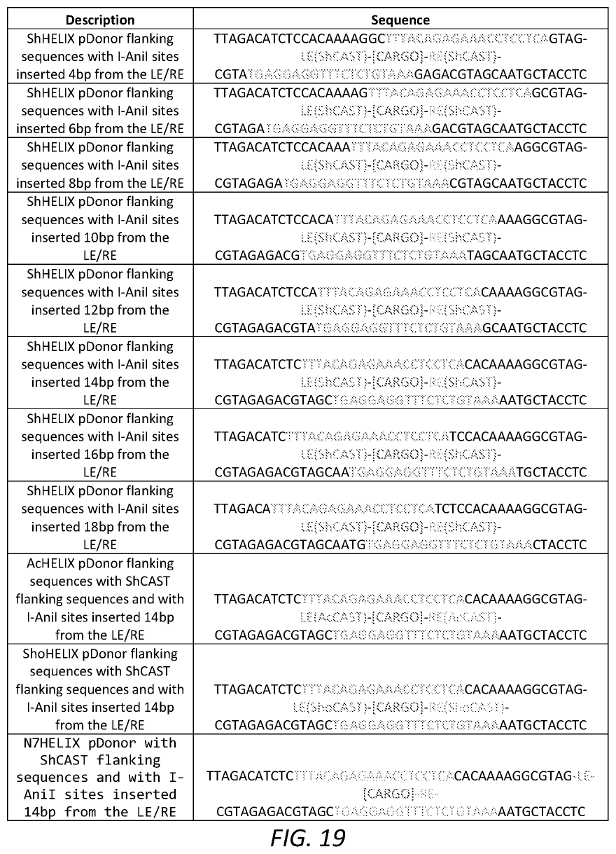

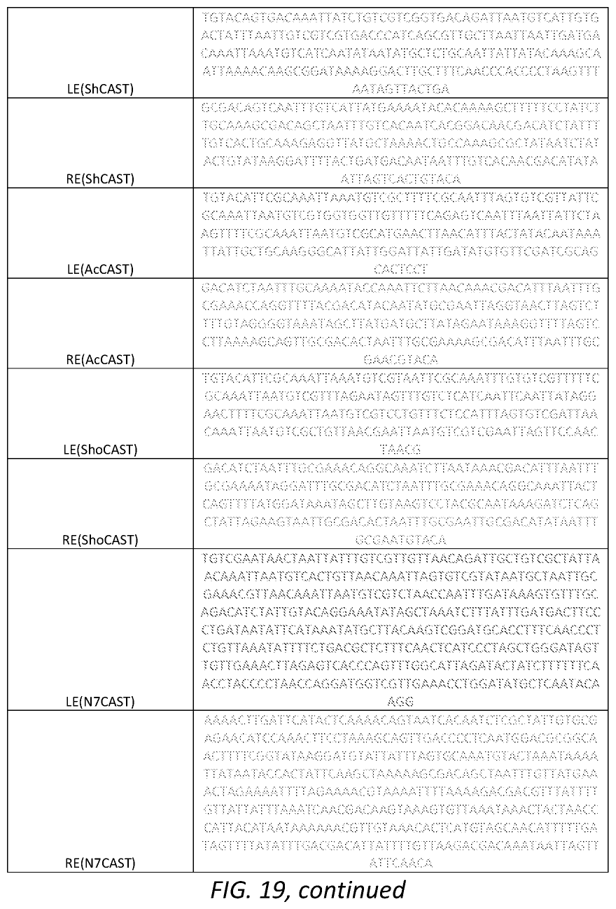

The ability to perform targeted DNA insertions in human cells has vast implications for basic research and therapeutics. To determine whether CAST or HELIX systems could function in human cells, we first determined whether ShCAST or AcCAST could function in a human context by attempting a lysate-based insertion assay. Plasmids encoding human codon-optimized CAST components were transfected into HEK 293T cells, incubated for 48 hours, and then lysed. The HEK 293T human cell lysate containing the CAST proteins was then incubated with pDonor, pTarget, and an in vitro transcribed sgRNA targeting TS1 on pTarget. However, for both ShCAST or AcCAST, we did not detect insertions into pTarget via junction PCR for the conditions tested. Next, given the generalizability of HELIX to various orthologs, we searched for other CASTs and identified the type V-K CAST from Nostoc Sp. PCC7101 (N₇CAST; FIG. 18a) that was previously shown to function in human cell lysate⁵⁰. After confirming that N₇CAST could demonstrate detectable DNA insertions an sgRNA against TS1 on pTarget in a HEK 293T cell lysate (FIG. 18b), we constructed an initial unoptimized N₇HELIX system (FIG. 5a and Example 10). Transposition experiments with N₇HELIX in lysates followed by junction PCRs on pTarget led to amplicons of the correct size (FIG. 5b, 5c), indicative of productive insertions. Sanger sequencing of these amplicons revealed donor insertion downstream of TS1 with expected target site duplications at the insertion site (FIG. 5d), and high-throughput sequencing revealed that insertions predominantly occurred 57-62 bp downstream of the PAM (FIG. 5e). To determine if N₇HELIX could improve desired insertion purity by decreasing cointegrate products relative to N₇CAST, we utilized a PCR enrichment strategy on our lysate reactions and employed long-read sequencing (Example 11). Whereas we observed 41.9% cointegrates with N₇CAST, equivalent experiments with N₇HELIX resulted in only 7.9% cointegrate products (a 5.3-fold decrease; FIG. 5f), indicating extensibility of HELIX into human cell contexts.

We then sought to streamline N₇HELIX for experiments in human cells by constructing a single all-in-one expression plasmid, while also varying the sequence of the sgRNA scaffold and the promoter (FIG. 18c and Example 10). When human cell lysate containing N₇HELIX expressed from the all-in-one plasmid was incubated with sgRNA2 (which contains mutated out poly-T stretches in the wild-type sgRNA to enable U6 promoter compatibility), pDonor, and pTarget, we observed sgRNA-dependent DNA insertion at TS1, validating that all components were active when expressed from a single plasmid (FIG. 18d). Next, we assessed whether N₇HELIX could mediate targeted DNA integration in human cells. We cotransfected pTarget and pDonor with plasmids encoding N₇CAST or N₇HELIX and either U6-sgRNA2 or CMV-driven wild type sgRNA flanked by a hammerhead and HDV ribozyme (FIG. 5g). However, no DNA integration was detected via junction PCR (FIG. 18e). Informed by recent work revealing that ribosomal S15 may be a crucial component of type V-K CASTs by facilitating complex assembly⁴³ (Example 10), we next attempted cotransfection of the same plasmids but now also including a plasmid encoding N₇S15 (FIG. 5g). Junction PCR across the left transposon end on extracted plasmid DNA revealed N₇CAST- or N₇HELIX-mediated donor integration on pTarget only when using N₇S15 and U6-sgRNA2 (FIG. 5h, FIG. 18e, and Example 10). Quantification of DNA insertions into pTarget revealed comparable integration between N₇CASTand N₇HELIX in the presence of N₇S15, albeit at low efficiencies (FIG. 5i). Given the structural and functional similarities between TnsB and TnsC in type V-K CASTs to MuA and MuB, respectively, of Mu transposon^{37,42} and the necessity of the host cofactor HU in Mu transposition¹, we next attempted transposition with N₇CAST or N₇HELIX along with cotrasfection of N₇S15 and an additional plasmid expressing N₇HU. Integration quantification showed similar efficiencies with or without HU coexpression (FIG. 5j). Next, experiments in HEK 293T cells targeting endogenous genomic target sites with N₇CAST or N₇HELIX and coexpression of N₇S15 (but not N₇HU) showed minimal, though detectable, insertions at VEGFA and EMX1 (FIG. 5k). Together, these results demonstrate the extensibility of HELIX into human cell contexts in the presence of S15 and motivate the continued development of CASTs and HELIX to achieve higher levels of integration in mammalian genomes (FIG. 5l).

### Example 7. Expanded Discussion of Y2 ShHELIX Results

While developing and characterizing ShHELIX, we also assessed whether the Y2 nAniI variant, previously shown to have a 9-fold higher affinity for its cognate target site¹, would enable a further increase in simple insertion product purity. With the Y2 ShHELIX construct, we observed a decrease in transformant colonies (FIG. 8a) when compared to ShCAST or non-Y2 ShHELIX (FIG. 6a). Moreover, this decrease varied with the spacing between the I-AniI site and LE/RE on pDonor, where a 14 bp spacing showed the highest number of colony-forming units (CFUs) (also aligning with the spacing giving the highest integration efficiency via ddPCR on plasmid and genomic targets). In combination with a similar observation when using a Lib4 I-AniI site (as shown in FIG. 1k), where the Lib4 I-AniI site was previously shown to increase wild type I-AniI affinity site by 5-fold², we recognized a potential correlation between the affinity of I-AniI for its target sequence and the number of colonies present on plates selecting for pShHELIX or pShCAST, pDonor and/or transposed product, and pTarget.

While further studies into the mechanism of HELIX will elucidate the basis of the decreased cell viability when using Y2-ShHELIX, we speculate that a combination of two phenomena may be occurring. First, the higher affinity of Y2 nAniI for its target, or when using nAniI with a Lib4 site, leads to an increased prevalence of DNA double-strand breaks (DSBs) on pDonor at early time points in the post-transformation recovery. In the absence of rapid and efficient cargo integration into pTarget, the AniI-caused DSBs result in a loss of Kanamycin resistance due to pDonor degradation prior to transposition. In this scenario, colony counts for different spacings on pDonor may correlate with higher or lower integration efficiencies. For example, for spacings where transposition is most efficient and rapid, the loss in CFUs is less striking because integration into pTarget occurs more rapidly than DSBs on pDonor. A second hypothesis is that the higher affinity of Y2 nAniI for its target, or when using nAniI with a Lib4 site, leads to an increased occurrence of DSBs on pDonor. Given the high copy number of pDonor in PIR1 cells, this could result in SOS response induction and cell death.

### Example 8. ShHELIX Control Experiments

While performing long-read sequencing of transposition products resulting from plasmid-targeting experiments, we included several control conditions. First, we performed experiments using a catalytically attenuated I-AniI variant (harboring K227M and Q171K mutations³) to create a ‘dead’ ShHELIX (dShHELIX). With dShHELIX, we observed a 1.8-fold decrease in co-integrate products compared to wild-type ShCAST (FIG. 9a and FIG. 1i, respectively). We hypothesize that this somewhat unexpected decrease in co-integrate products is the result of incomplete inactivation of I-AniI catalysis, which might lead to low-level 5′ pDonor nicking (at a rate slower than nAniI-based ShHELIX). Indeed, the I-AniI Q171K variant has previously been shown to exhibit residual nicking activity on both DNA strands in vitro³.

Secondly, we performed experiments using a pDonor variant that does not harbor I-AniI sites. In transformations with ShHELIX and this modified pDonor lacking I-AniI sites, we observed a 1.7-fold decrease in co-integrates relative to ShCAST (FIG. 9a and FIG. 1i, respectively). We hypothesize that this could be due to low-level I-AniI activity on sequences flanking the LE and RE (where tethering to TnsB induces energetically unfavorable interactions that would not occur in the absence of the fusion). A previous study that mutated each base in the I-AniI recognition sequence to all other bases revealed that specificity of nAniI is greatest across base pair positions ±3, 4, 5, and 6 in each half-site and least specific across bases −2 to +1 and bases at the outer edges of the recognition sequence³. From this data, a minimal approximate core sequence of 5′-GAGGNNNCTCTG-3′ is necessary for I-AniI recognition, with decreased activity depending on the base substituted. While we could not identify an exact sequence match, we note that sequences similar to these core motifs occur on pDonor at 5′-GTGGNNNNGTCTA-3′ (11 bp from the LE) and 5′-GAGGNNNCATTG-3′ (13 bp from the RE), the latter being in an orientation that would give a nick on the same strand as TnsB (see next point). Low-level nicking on these flanking sequences at these degenerate I-AniI core sequences might lead to a slight increase in simple insertion product purity (as observed).

Thirdly, we performed experiments using ‘flipped’ I-AniI sites on pDonor oriented to confer a nick on the same strand as TnsB. In experiments using a flipped I-AniI site pDonor, we observed a 10-fold decrease in co-integrates with ShHELIX relative to ShCAST (FIG. 9b). We hypothesize that this reduction in co-integrates might be the result of an alternative transposition mechanism involving 5′ flap cleavage of the gapped Shapiro intermediate (FIG. 9c).

### Example 9. Mechanistic Implications of Cas12k-TnsC Fusions

Recent structural studies have provided insight into the mechanism of ShCAST-mediated DNA insertion⁴⁻⁶. These studies suggest that TnsB recruitment to TniQ-nucleated TnsC filaments simulates filament disassembly, exposing the target site and inducing insertion at a coordinated distance from the sgRNA-Cas12k-DNA complex. Our experiments with fusions of Cas12k to a TnsC monomer in the context of ShCAST or ShHELIX (FIG. 3) are interesting given these proposed mechanisms, particularly regarding the role of TnsC filamentation in recruiting downstream transposition machinery. Additionally, since the extent of TnsC filament disassembly (or the footprint of TniQ alone or bound to TnsC) may define the insertion distance from bound DNA-bound Cas12k for canonical 4-component ShCAST, it is interesting that Cas12k-TnsC fusions (in the context of ShCAST and ShHELIX systems) enable targeted DNA insertion with the same insertion distance profiles as the canonical 4-component ShCAST and ShHELIX systems (FIG. 12). We speculate that TnsC filamentation may still occur, despite Cas12k fusion, or that only a single TnsC subunit fused to Cas12k is sufficient to enable transposition. In the latter case, it is possible that TnsB-mediated depolymerization collapses TnsC filaments to a single monomer, which results in the fixed insertion distance profile observed for natural systems and would align with the identical profile observed for our monomer fusion. Alternatively, TnsC may not be involved in insertion distance determination, and a TniQ and TnsB defined insertion distance model may be more plausible. However, the molecular ruler mechanism of CASTs is still unclear. Furthermore, ShCAST our results revealed that a Cas12k-TniQ-TnsC fusion is functional (albeit with reduced activity) whereas a Cas12k-TnsC-TniQ fusion completely abolished activity (FIG. 4b). This observation may support the current model where Cas12k and TniQ must be able to directly interact⁵. Our results with Cas12k-TnsC and Cas12k-TniQ-TnsC fusions provide insight into the role of TnsC and TniQ in ShCAST-mediated transposition, motivating further studies to elucidate the transposition mechanism of both natural CASTs and engineered HELIX 2-, 3-, or 4-component systems.

### Example 10. Construction and Characterization of N₇HELIX in Human Cell Contexts

To construct N₇HELIX, a human codon optimized nicking variant of I-AniI was fused to N₇TnsB via an 18 amino acid XTEN linker. I-AniI sites were positioned 14 bp from the LE and RE on pDonor in the correct orientation to confer a 5′ nick, and the flanking sequences directly adjacent to the LE and RE were swapped for those of ShCAST (FIG. 5a). Although this donor flank configuration was most efficient for ShHELIX, it is possible that N₇-specific optimizations for N₇HELIX might yield higher integration efficiencies. To streamline N₇HELIX expression, we constructed a single all-in-one plasmid where all four HELIX components were driven by a single CMV promoter as previously described⁷. Specifically, NLS-Cas12k and TnsC as well as NLS-nAniI-TnsB and NLS-TniQ were linked by T2A sequences. Polypeptide pairs were separated by an EMCV internal ribosome entry site (IRES) (FIG. 17c). We also generated a modified version of the sgRNA (sgRNA2) with substitutions in several poly-T stretches within the scaffold of the wild-type sgRNA (which can serve as termination signal for the U6 promoter⁸) (FIG. 17c).

Recent work has demonstrated that host-encoded ribosomal protein S15 in bacteria is a bona fide component of type V-K CASTs, allosterically stimulating complex assembly at the Cas12k-bound target site⁵. Remarkably, the ShCAST sgRNA scaffold secondary structure to which S15 was found to be bound is strikingly similar to that of 16S rRNA (which S15 binds in its primary role in facilitating ribosomal complex assembly). Both E. coli S15 (EcS15) and S. Hofmannii S15 (ShS15) were previously shown to substantially enhance transposition in vitro⁵. Due to these observations, we generated expression plasmids for both N₇ ribosomal protein S15 (N₇S15) and EcS15 to determine if they could promote N₇CAST and N₇HELIX (FIG. 5g, 5h, and FIG. 18e). We found that N₇S15 coexpression was required for N₇CAST and N₇HELIX integration in human cells (FIG. 18e), corroborating prior findings⁵ that S15 is likely needed for optimal targeted integration and that it should be heterologously expressed when type V-K CASTs or HELIX is used in human cells. Under the conditions that we examined, we did not observe N₇CAST and N₇HELIX integration in human cells when EcS15 was coexpressed (FIG. 18e).

Despite detection of CAST- and HELIX-mediated transposition in human cells when expressing S15, overall insertion efficiency remained low for constructs and conditions tested. As expanded upon in our main text, discovering additional required host factors implicated in type V-K CAST function as well as screening for type V-K CAST orthologs that may be naturally suited for a human cell context will be needed. Directed evolution of CAST systems, particularly TnsB and Cas12k, and structure-guided engineering may enable more efficient integration on human genomic targets. Continued optimization of protein and sgRNA expression constructs and methods will also prove important given the complexity of these systems and the requirement to localize all components to the nucleus. Optimized component fusions may prove useful to help facilitate nuclear localization.

It should also be noted that the HELIX architectures may require optimization for each CAST ortholog. These optimizations include: spacing between the I-AniI site and LE/RE, linkers between nAniI and TnsB or between other components (if applicable), the identity of the LHE itself, and flanking sequences on the donor. System specific optimizations were not conducted for the other orthologs described in this study (AcCAST, ShoCAST, and N₇CAST), as we designed and constructed N₇HELIX according to the optimal parameters from our ShHELIX/AcHELIX experiments. Therefore, ortholog-specific optimizations may enable more efficient HELIX-mediated human genome targeting.

### Example 11. Cointegrate Characterization from Experiments in HEK 293T Cell Lysates

We explored the extensibility of HELIX to reduce cointegrates relative to its canonical CAST in human cell contexts. Due to low efficiency transposition in human lysates with the constructs and conditions that we examined, the enrichment process that we utilized for bacterial plasmid-targeting experiments was not feasible or applicable for experiments conducted in human lysate. Therefore, we opted to utilize a PCR-based enrichment strategy from the lysate reaction to quantify the approximate proportion of simple insertions to cointegrate products (see diagram below). Two separate 20-cycle PCRs each using an identical volume of terminated lysate reaction as template were conducted that differed only by the sequence of the downstream reverse primer. The PCRs sought to: (A) amplify from upstream of TS1 on pTarget to the edge of the RE on the inserted cargo (to approximate ‘total’ insertions), and (B) amplify from upstream of TS1 on pTarget (same 5′ primer as first PCR reaction) to donor backbone near the edge of the RE. Both PCRs were performed for CAST and HELIX, the PCRs were combined and analyzed via long-read sequencing as described in methods. Reads from PCR-A represent “total” insertions whereas reads from PCR-B represent “cointegrate” insertions. The ratio of “cointegrate” to “total insertions” was used to estimate the relative proportion of cointegrates from total transposed product, albeit an approximate quantification and meant only to compare the relative differences between CAST and HELIX.

### Exemplary Sequences

NOTE: Sequences will vary for each different CAST system to which HELIX is applied. For those used in this study, see below:

ShCAST subunits
ShCAST Cas12k
(SEQ ID NO: 68)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRS
ShCAST TnsB
(SEQ ID NO: 69)
MNSQQNPDLAVHPLAIPMEGLLGESATTLEKNVIATQLSEEAQVKLEVIQ
SLLEPCDRTTYGQKLREAAEKLNVSLRTVQRLVKNWEQDGLVGLTQTSRADKG
KHRIGEFWENFITKTYKEGNKGSKRMTPKQVALRVEAKARELKDSKPPNYKTVL
RVLAPILEKQQKAKSIRSPGWRGTTLSVKTREGKDLSVDYSNHVWQCDHTRVD
VLLVDQHGEILSRPWLTTVIDTYSRCIMGINLGFDAPSSGVVALALRHAILPKRYG
SEYKLHCEWGTYGKPEHFYTDGGKDFRSNHLSQIGAQLGFVCHLRDRPSEGGVV
ERPFKTLNDQLFSTLPGYTGSNVQERPEDAEKDARLTLRELEQLLVRYIVDRYNQ
SIDARMGDQTRFERWEAGLPTVPVPIPERDLDICLMKQSRRTVQRGGCLQFQNL
MYRGEYLAGYAGETVNLRFDPRDITTILVYRQENNQEVFLTRAHAQGLETEQLA
LDEAEAASRRLRTAGKTISNQSLLQEVVDRDALVATKKSRKERQKLEQTVLRSA
AVDESNRESLPSQIVEPDEVESTETVHSQYEDIEVWDYEQLREEYGF
ShCAST TnsC
(SEQ ID NO: 70)
MTEAQAIAKQLGGVKPDDEWLQAEIARLKGKSIVPLQQVKTLHDWLDG
KRKARKSCRVVGESRTGKTVACDAYRYRHKPQQEAGRPPTVPVVYIRPHQKCG
PKDLFKKITEYLKYRVTKGTVSDFRDRTIEVLKGCGVEMLIIDEADRLKPETFAD
MRDIAEDLGIAVVLVGTDRLDAVIKRDEQVLERFRAHLRFGKLSGEDFKNTVEM
WEQMVLKLPVSSNLKSKEMLRILTSATEGYIGRLDEILREAAIRSLSRGLKKIDKA
VLQEVAKEYK
ShCAST TniQ
(SEQ ID NO: 71)
MIEAPDVKPWLFLIKPYEGESLSHFLGRFRRANHLSASGLGTLAGIGAIVA
RWERFHFNPRPSQQELEAIASVVEVDAQRLAQMLPPAGVGMQHEPIRLCGACYA
ESPCHRIEWQYKSVWKCDRHQLKILAKCPNCQAPFKMPALWEDGCCHRCRMPF
AEMAKLQKV
ShCAST sgRNA scaffold ribonucleotide
(SEQ ID NO: 72)
AUAUUAAUAGCGCCGCAAUUCAUGCUGCUUGCAGCCUCUGAAUUUU
GUUAAAUGAGGGUUAGUUUGACUGUAUAAAUACAGUCUUGCUUUCUGACC
CUGGUAGCUGCUCACCCUGAUGCUGCUGUCAAUAGACAGGAUAGGUGCGC
UCCCAGCAAUAAGGGCGCGGAUGUACUGCUGUAGUGGCUACUGAAUCACC
CCCGAUCAAGGGGGAACCCUAAAUGGGUUGAAAG
AcCAST Cas12k amino acid
(SEQ ID NO: 73)
MSVITIQCRLVAEEDSLRQLWELMSEKNTPFINEILLQIGKHPEFETWLEK
GRIPAELLKTLGNSLKTQEPFTGQPGRFYTSAITLVDYLYKSWFALQKRRKQQIE
GKQRWLKMLKSDQELEQESQSSLEVIRNKATELFSKFTPQSDSEALRRNQNDKQ
KKVKKTKKSTKPKTSSIFKIFLSTYEEAEEPLTRCALAYLLKNNCQISELDENPEEF
TRNKRRKEIEIERLKDQLQSRIPKGRDLTGEEWLETLEIATFNVPQNENEAKAWQ
AALLRKTANVPFPVAYESNEDMTWLKNDKNRLFVRFNGLGKLTFEIYCDKRHL
HYFQRFLEDQEILRNSKRQHSSSLFTLRSGRIAWLPGEEKGEHWKVNQLNFYCSL
DTRMLTTEGTQQVVEEKVTAITEILNKTKQKDDLNDKQQAFITRQQSTLARINNP
FPRPSKPNYQGKSSILIGVSFGLEKPVTVAVVDVVKNKVIAYRSVKQLLGENYNL
LNRQRQQQQRLSHERHKAQKQNAPNSFGESELGQYVDRLLADAIIAIAKKYQAG
SIVLPKLRDMREQISSEIQSRAENQCPGYKEGQQKYAKEYRINVHRWSYGRLIESI
KSQAAQAGIAIETGKQSIRGSPQEKARDLAVFTYQERQAALI
AcCAST TnsB
(SEQ ID NO: 74)
MADEEFEFTEGTTQVPDAILLDKSNFVVDPSQIILATSDRHKLTFNLIQWL
AESPNRTIKSQRKQAVANTLDVSTRQVERLLKQYDEDKLRETAGIERADKGKYR
VSEYWQNFITTIYEKSLKEKHPISPASIVREVKRHAIVDLELKLGEYPHQATVYRIL
DPLIEQQKRKTRVRNPGSGSWMTVVTRDGELLRADFSNQIIQCDHTKLDVRIVD
NHGNLLSDRPWLTTIVDTFSSCVVGFRLWIKQPGSTEVALALRHAILPKNYPEDY
QLNKSWDVCGHPYQYFFTDGGKDFRSKHLKAIGKKLGFQCELRDRPPEGGIVER
IFKTINTQVLKELPGYTGANVQERPENAEKEACLTIQDLDKILASFFCDIYNHEPY
PKEPRDTRFERWFKGMGGKLPEPLDERELDICLMKEAQRVVQAHGSIQFENLIYR
GEFLKAHKGEYVTLRYDPDHILSLYIYSGETDDNAGEFLGYAHAVNMDTHDLSI
EELKALNKERSNARKEHFNYDALLALGKRKELVEERKEDKKAKRNSEQKRLRS
ASKKNSNVIELRKSRTSKSLKKQENQEVLPERISREEIKLEKIEQQPQENLSASPNT
QEEERHKLVFSNRQKNLNKIW
AcCAST TnsC
(SEQ ID NO: 75)
MAQPQLATQSIVEVLAPRLDIKAQIAKTIDIEEIFRACFITTDRASECFRWL
DELRILKQCGRIIGPRNVGKSRAALHYRDEDKKRVSYVKAWSASSSKRLFSQILK
DINHAAPTGKRQDLRPRLAGSLELFGLELVIIDNAENLQKEALLDLKQLFEECNV
PIVLAGGKELDDLLHDCDLLTNFPTLYEFERLEYDDFKKTLTTIELDVLSLPEASN
LAEGNIFEILAVSTEARMGILIKILTKAVLHSLKNGFHRVDESILEKIASRYGTKYIP
LKNRNRD
AcCAST TniQ
(SEQ ID NO: 76)
MAQNIFLSKTEIGIDEDDEIRPKLGYVEPYEEESISHYLGRLRRFKANSLPS
GYSLGKIAGLGAMISRWEKLYFNPFPTLQELEALSSVVGVNADRLIEMLPSQGMT
MKPRPIRLCGACYAESPCHRIEWQCKDRMKCDRHNLRLLIKCTNCETPFPIPADW
VKGQCPHCSLPFAKMAKRQRRD
AcCAST sgRNA scaffold
(SEQ ID NO: 77)
AUAUGGAUACAACAGCGCCGUAGUUCAUGCUCCUUGGAGUCUCUGU
ACUAUGAAAAAUCUGGCUUAGUUUGGCAGUUGGAAGACUGUCAUGCUUUC
UGAGCCUGGUAGCUGCCCGCUUCUGAUGCUGCUGUCGCAAGACAGGAUAG
GUGCGCUCCCAGCAAUAAGGAGUAAGGCUUUUAGCCAUAGUCGUUAUUUA
UAACGAUGUGGAUUUCCACAGUGGUGGCUACUGAAUCACCCCCUUCGUCG
GGGGAACCCUAAAUGGGUUGAAAG
ShoCAST Cas12k
(SEQ ID NO: 78)
MSTITIQCRLVAEEATLRYFWELMAEKNTPLINELLEQLGQHPDFDTWVQ
AGKMPEKTVENLCKSLEDREPFANQPGRFRTSAVALVKYIYKSWFALQKRRAD
RLEGKERWLKMLKSDVELERESNCSLDIIRAKAGEILAKVTEGCAPSNQTSSKRK
KKKTKKSQATKDLPTLFEIILKAYEQAEESLTRAALAYLLKNDCEVSEVDEDSEK
FKKRRRKKEIEIERLRNQLKSRIPKGRDLTGDKWLKTLEEATRNVPENEDEAKA
WQAQLLREASSVPFPVAYETSEDMTWFTNEQGRIFVYFNGSAKHKFQVYCDRR
QLHWFQRFVEDFQIKKNGDKKGSEKEYPAGLLTLCSTRLRWKESAEKGDPWNV
HRLILSCTIDTRLWTLEGTEQVRAEKIAQVEKTISKREQEVNLSKTQLERLQAKHS
ERERLNNIFPNRPSKPSYRGKSHIAIGVSFSLENPATVAVVDVATKKVLTYRSFKQ
LLGDNYNLANRLRQQKQRLSHERHKAQKQGAPNSFGDSELGQYVDRLLAKSIV
AIAKTYQASSIVLPKLRYMREIIHNEVQAKAEKKIPGYKEGQKQYAKQYRISVHQ
WSYNRLSQILESQATKAGISIERGSQVIQGSSQEQARDLALFAYNERQLSLG
ShoCAST TnsB
(SEQ ID NO: 79)
MGLDEEFEFTEELTQAPDVIVLDKSHFVVDPSQIILQTSDKHKLRFNLIKW
FAESPNITIKSQRKQAVVDTLGVSTRQVERLLKQYHNGELSETAGVQRSDKGKL
RISQYWEDYIKTTYEKSLKDKHPMLPAAVVREVKRHAIVDLGLKPGDYPHPATI
YRNLAPLIEQHTRKKKVRNPGSGSWLTVVTRDGQLLKADFSNQIIQCDHTELDIH
IVDSHGSLLSDRPWLTTVVDTYSSCILGFHLWIKQPGSTEVALALRHAILPKNYPE
DYKLGKVWEIYGPPFQYFFTDGGKDFNSKHLKAIGKKLGFQCELRNRPPQGGIV
ERLFKTINTQVLKELPGYTGANVQERPKNAEKEACLTIQDLDKILASFFCDIYNHE
PYPKEPRNTRFERWFKGMGGKLPEPLDERELDICLMKEAQRVVQAHGSIQFENLI
YRGEALKAYRGEYVTLRYDPDHVLTLYVYSCEADDNAEEFLGYAHAINMDTHD
LSIEELKTLNKERSKARSDHYNYDALLALGKRKELVEERKQDKKAKRQSEQKRL
RTASKKNSNVIELRKSRASSSSSKDDRQEILPERVSRDELKPEKTELKYEENLLAQ
TDTQKQERHKLVVSDRKKNLKNIW
ShoCAST TnsC
(SEQ ID NO: 80)
MAISQLATQPFVEVLPPELDSKAQIAKTIDIEELFRINFITTDRSSECFRWLD
ELRILKQCGRIIGPRNVGKSRAVLHYRNEDKKRVSYVKAWSASSSKRLFSQILKD
INHAASTGKRQDLRPRLAGSLELFGLELVIVDNAENLQKEALLDLKQLFEECHVP
IVLVGGKELDDILEDFDLLTNFPTLYEFERLEHDDFIKTLKTIELDILSLPEASKLSE
GNIFAILAESTGGKIGILVKILTKAVLHSLKKGFGKVDESILEKIASRYGTKYVPIE
NKNRND
ShoCAST TniQ
(SEQ ID NO: 81)
MIEDDEIRLRLGYVEPHPGESISHYLGRLRRFKANSLPSGYALGKIAGLGS
VLTRWEKLYFNPFPTQQELEALAQVIQVEVEKLREMLPTKGVTMMPRPIRLCAA
CYAESPYHRIEWQFKDKMKCDRHQLRLLTKCTNCQTPFPIPADWEKGECSHCFL
SFAKMVKCQKRR
ShoCAST sgRNA scaffold
(SEQ ID NO: 82)
GGGUACUAAUAGCGCCGCAGUUCAUGCUCUUUAAGAGUCUCUGUAC
UGUGGAAAAUCUGGGUUAGUUUGACGGUUGGAAAACCGUUUUGCUUUCUG
ACCCUGGUAGCUGCCCGCUUCUCAUGCUCUGACUUUUCACGUUAUGUGGA
AAAAGUAACGUAAUUUCGUUAGUUAAGACUUACCGUAAAAAGUCAGUUCU
GAUGCUGCUGUCGCAAGACAGGAUAGGUGCGCUCCCAGCAAAAGGAGUAU
GUCUUGAAAAAGACUAGCCGUUCUAGUAACGGUGCGGAUUACCGCAGUGG
UGGCUACUGAAUCACCCCCUUCGUCGGGGGAACCCUCCAAAAGGUGGGUU
GAAAG
N₇CAST Cas12k
(SEQ ID NO: 83)
MSVITIQCRLVAEEDILRQLWELMADKNTPLINELLAQVGKHPEFETWLD
KGRIPTKLLKTLVNSFKTQERFADQPGRFYTSAIALVDYVYKSWFALQKRRKRQI
EGKERWLTILKSDLQLEQESQCSLSAIRTKANEILTQFTPQSEQNKNQRKGKKTK
KSTKSEKSSLFQILLNTYEQTQNPLTRCAIAYLLKNNCQISELDEDSEEFTKNRRK
KEIEIERLKNQLQSRIPKGRDLTGEEWLKTLEISTANVPQNENEAKAWQAALLRK
SADVPFPVAYESNEDMTWLQNDKGRLFVRFNGLGKLTFEIYCDKRHLHYFKRFL
EDQELKRNHKNQYSSSLFTLRSGRLAWSPGEEKGEPWKVNQLHLYCTLDTRMW
TIEGTQQVVDEKSTKINETLTKAKQKDDLNDQQQAFITRQQSTLDRINNLFPRPSK
SRYQGQPSILVGVSFGLKKPVTVAVVDVVKNEVLAYRSVKQLLGENYNLLNRQ
RQQQQRLSHERHKAQKQNAPNSFGESELGQYIDRLLADAIIAIAKTYQAGSIVLP
KLRDMREQISSEIQSRAEKKCPGYKEVQQKYAKEYRMSVHRWSYGRLIECIKSQ
AAKAGISTEIGTQPIRGSPQEKARDVAVFAYQERQAALI
N₇CAST TnsB
(SEQ ID NO: 84)
MDEMPIVKQDDESLPVENNDDVDEIQDDELEETNVIFTELSAEAKLKMDV
IQGLLEPCDRKTYGEKLRVAAEKLGKTVRTVQRLVKKYQQDGLSAIVETQRNDK
GSYRIDPEWQKFIVNTFKEGNKGSKKMTPAQVAMRVQVRAEQLGLQKFPSHMT
VYRVLNPIIERQERKQKQRNIGWRGSRVSHKTRDGQTLDVRYSNHVWQCDHTK
LDVMLVDQYGEPLARPWFTKITDSYSRCIMGIHVGFDAPSSQVVALASRHAILPK
QYSAEYKLISDWGTYGVPENLFTDGGRDFRSEHLKQIGFQLGFECHLRDRPSEGG
IEERSFGTINTEFLSGFYGYLGSNIQERSKTAEEEACLTLRELHLLLVRYIVDNYNQ
RLDARTKDQTRFQRWEAGLPALPKMVKERELDICLMKKTRRSIYKGGYLSFENI
MYRGDYLAAYAGENIVLRYDPRDITTVWVYRIDKGKEVFLSAAHALDWETEQL
SLEEAKAASRKVRSVGKTLSNKSILAEIHDRDTFIKQKKKSQKERKKEEQAQVHA
VYEPINLSETEPLENLQETPKPVTRKPRIFNYEQLRQDYDE
N₇CAST TnsC
(SEQ ID NO: 85)
MKDDYWQRWVQNLWGDEPIPEELQPEIERLLSPSVVELEHIQKIHDWLD
GLRLSKQCGRIVAPPRAGKSVTCDVYRLLNKPQKRGGKRDIVPVLYMQVPGDCS
SGELLVLILESLKYDATSGKLTDLRRRVQRLLKESKVEMLIIDEANFLKLNTFSEI
ARIYDLLRISIVLVGTDGLDNLIKREPYIHDRFIECYKLPLVESEKKFTELVKIWEE
EVLCLPLPSNLTRSETLEPLRRKTGGKIGLVDRVLRRASILALRKGLKNIDKETLT
EVLDWFE
N₇CAST TniQ
(SEQ ID NO: 86)
MEIGAEEPHIFEVEPLEGESLSHFLGRFRRENYLTSSQLGKLTGLGAVVSR
WKKLYFNPFPTRQELEALTSVVRVNADRLAEMLPPKGVTMKPRPIRLCAACYAE
VPCHRIEWQFKDVMKCDRHNLRLLTKCTNCETSFPIPAEWVQGECPHCFLPFAT
MAKRQKHG
N₇CAST sgRNA scaffold (wild type sequence)
(SEQ ID NO: 87)
AUAUUUUUAUAACAGCGCCGCAGUUCAUGCUUUUUUAAGCCAAUGU
ACUGUGAAAAAUCUGGGUUAGUUUGGCGGUUGGAAGGCCGUCAUGCUUUC
UGACCCUUGUAGCUGCCCGCUUCUGAUGCUGCCAUCUUUAGAAUUCUAUA
GGUGGGAUAGGUGCGCUCCCAGCAAUAAGGAGUAAGGCUUUUAGCUAUAG
CCGUUAUUCAUAACGGUGCGGAUUACCACAGUGGUGGCUACUGAAUCACC
CCCUUCGUCGGGGGAACCCUCCAAAAGGUGGGUUGAAAG
N₇CAST sgRNA scaffold (poly-U stretches in wild-type scaffold mutated to
reduce or prevent premature transcriptional termination)
(SEQ ID NO: 88)
AUAUUCUUAUAACAGCGCCGCAGUUCAUGCUUUCUUAAGCCAAUGU
ACUGUGAAAAAUCUGGGUUAGUUUGGCGGUUGGAAGGCCGUCAUGCUUUC
UGACCCUUGUAGCUGCCCGCUUCUGAUGCUGCCAUCUUUAGAAUUCUAUA
GGUGGGAUAGGUGCGCUCCCAGCAAUAAGGAGUAAGGCUUAUAGCUAUAG
CCGUUAUUCAUAACGGUGCGGAUUACCACAGUGGUGGCUACUGAAUCACC
CCCUUCGUCGGGGGAACCCUCCAAAAGGUGGGUUGAAAG
I-AniI and variants:
Wild type I-AniI amino acid sequence
(SEQ ID NO: 89)
MGSDLTYAYLVGLFEGDGYFSITKKGKYLTYELGIELSIKDVQLIYKIKKIL
GIGIVSFRKRNEIEMVALRIRDKNHLKSFILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNKKLQYLLWLKQLRKISRYSEKIKIPSNY
I-AniI amino acid sequence containing two mutations (F80K, L232K) conferring
increased solubility/solution behavior
(SEQ ID NO: 90)
MGSDLTYAYLVGLFEGDGYFSITKKGKYLTYELGIELSIKDVQLIYKIKKIL
GIGIVSFRKRNEIEMVALRIRDKNHLKSKILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNKKLQYKLWLKQLRKISRYSEKIKIPSNY
Nicking variant of I-AniI amino acid sequence (also containing the solution behavior
mutations, F80K, L232K, K227M)
(SEQ ID NO: 91)
MGSDLTYAYLVGLFEGDGYFSITKKGKYLTYELGIELSIKDVQLIYKIKKIL
GIGIVSFRKRNEIEMVALRIRDKNHLKSKILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNMKLQYKLWLKQLRKISRYSEKIKIPSNY
Y2 I-AniI-amino acid sequence harboring two additional mutations shown to increase
affinity 9-fold (F80K, L232K, F13Y, S111Y)
(SEQ ID NO: 92)
MGSDLTYAYLVGLYEGDGYFSITKKGKYLTYELGIELSIKDVQLIYKIKKI
LGIGIVSFRKRNEIEMVALRIRDKNHLKSKILPIFEKYPMFSNKQYDYLRFRNALL
SGIIYLEDLPDYTRSDEPLNSIESIINTSYFSAWLVGFIEAEGCFSVYKLNKDDDYLI
ASFDIAQRDGDILISAIRKYLSFTTKVYLDKTNCSKLKVTSVRSVENIIKFLQNAPV
KLLGNKKLQYKLWLKQLRKISRYSEKIKIPSNY
Nicking variant of Y2 I-AniI amino acid sequence (F80K, L232K, K227M, F13Y, S111Y)
(SEQ ID NO: 93)
MGSDLTYAYLVGLYEGDGYFSITKKGKYLTYELGIELSIKDVQLIYKIKKI
LGIGIVSFRKRNEIEMVALRIRDKNHLKSKILPIFEKYPMFSNKQYDYLRFRNALL
SGIIYLEDLPDYTRSDEPLNSIESIINTSYFSAWLVGFIEAEGCFSVYKLNKDDDYLI
ASFDIAQRDGDILISAIRKYLSFTTKVYLDKTNCSKLKVTSVRSVENIIKFLQNAPV
KLLGNMKLQYKLWLKQLRKISRYSEKIKIPSNY
TnsB fusions (expressed with TnsC, TniQ, Cas12k in HELIX systems)
nAniI-XTEN18-ShTnsB : nicking I-AniI fused to ShCAST TnsB with an 18 amino acid
XTEN linker
(SEQ ID NO: 94)
MGSDLTYAYLVGLFEGDGYFSITKKGKYLTYELGIELSIKDVQLIYKIKKIL
GIGIVSFRKRNEIEMVALRIRDKNHLKSKILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNMKLQYKLWLKQLRKISRYSEKIKIPSNYSGSETPGTSESATPESGSNSQQNP
DLAVHPLAIPMEGLLGESATTLEKNVIATQLSEEAQVKLEVIQSLLEPCDRTTYGQ
KLREAAEKLNVSLRTVQRLVKNWEQDGLVGLTQTSRADKGKHRIGEFWENFIT
KTYKEGNKGSKRMTPKQVALRVEAKARELKDSKPPNYKTVLRVLAPILEKQQK
AKSIRSPGWRGTTLSVKTREGKDLSVDYSNHVWQCDHTRVDVLLVDQHGEILSR
PWLTTVIDTYSRCIMGINLGFDAPSSGVVALALRHAILPKRYGSEYKLHCEWGTY
GKPEHFYTDGGKDFRSNHLSQIGAQLGFVCHLRDRPSEGGVVERPFKTLNDQLFS
TLPGYTGSNVQERPEDAEKDARLTLRELEQLLVRYIVDRYNQSIDARMGDQTRF
ERWEAGLPTVPVPIPERDLDICLMKQSRRTVQRGGCLQFQNLMYRGEYLAGYA
GETVNLRFDPRDITTILVYRQENNQEVFLTRAHAQGLETEQLALDEAEAASRRLR
TAGKTISNQSLLQEVVDRDALVATKKSRKERQKLEQTVLRSAAVDESNRESLPS
QIVEPDEVESTETVHSQYEDIEVWDYEQLREEYGF
Y2 nAniI-XTEN18-ShInsB: nicking I-AniI fused to ShCAST TnsB with an 18 amino acid
XTEN linker
(SEQ ID NO: 95)
MGSDLTYAYLVGLYEGDGYFSITKKGKYLTYELGIELSIKDVQLIYKIKKI
LGIGIVSFRKRNEIEMVALRIRDKNHLKSKILPIFEKYPMFSNKQYDYLRFRNALL
SGIIYLEDLPDYTRSDEPLNSIESIINTSYFSAWLVGFIEAEGCFSVYKLNKDDDYLI
ASFDIAQRDGDILISAIRKYLSFTTKVYLDKTNCSKLKVTSVRSVENIIKFLQNAPV
KLLGNMKLQYKLWLKQLRKISRYSEKIKIPSNYSGSETPGTSESATPESGSNSQQN
PDLAVHPLAIPMEGLLGESATTLEKNVIATQLSEEAQVKLEVIQSLLEPCDRTTYG
QKLREAAEKLNVSLRTVQRLVKNWEQDGLVGLTQTSRADKGKHRIGEFWENFI
TKTYKEGNKGSKRMTPKQVALRVEAKARELKDSKPPNYKTVLRVLAPILEKQQ
KAKSIRSPGWRGTTLSVKTREGKDLSVDYSNHVWQCDHTRVDVLLVDQHGEILS
RPWLTTVIDTYSRCIMGINLGFDAPSSGVVALALRHAILPKRYGSEYKLHCEWGT
YGKPEHFYTDGGKDFRSNHLSQIGAQLGFVCHLRDRPSEGGVVERPFKTLNDQL
FSTLPGYTGSNVQERPEDAEKDARLTLRELEQLLVRYIVDRYNQSIDARMGDQT
RFERWEAGLPTVPVPIPERDLDICLMKQSRRTVQRGGCLQFQNLMYRGEYLAGY
AGETVNLRFDPRDITTILVYRQENNQEVFLTRAHAQGLETEQLALDEAEAASRRL
RTAGKTISNQSLLQEVVDRDALVATKKSRKERQKLEQTVLRSAAVDESNRESLP
SQIVEPDEVESTETVHSQYEDIEVWDYEQLREEYGF
nAniI-XTEN18-AcTnsB: nicking I-AniI (as in row 26) fused to AcCAST TnsB with an 18
amino acid XTEN linker
(SEQ ID NO: 96)
MGSDLTYAYLVGLFEGDGYFSITKKGKYLTYELGIELSIKDVQLIYKIKKIL
GIGIVSFRKRNEIEMVALRIRDKNHLKSKILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNMKLQYKLWLKQLRKISRYSEKIKIPSNYSGSETPGTSESATPESGSADEEFE
FTEGTTQVPDAILLDKSNFVVDPSQIILATSDRHKLTFNLIQWLAESPNRTIKSQRK
QAVANTLDVSTRQVERLLKQYDEDKLRETAGIERADKGKYRVSEYWQNFITTIY
EKSLKEKHPISPASIVREVKRHAIVDLELKLGEYPHQATVYRILDPLIEQQKRKTR
VRNPGSGSWMTVVTRDGELLRADFSNQIIQCDHTKLDVRIVDNHGNLLSDRPWL
TTIVDTFSSCVVGFRLWIKQPGSTEVALALRHAILPKNYPEDYQLNKSWDVCGHP
YQYFFTDGGKDFRSKHLKAIGKKLGFQCELRDRPPEGGIVERIFKTINTQVLKELP
GYTGANVQERPENAEKEACLTIQDLDKILASFFCDIYNHEPYPKEPRDTRFERWF
KGMGGKLPEPLDERELDICLMKEAQRVVQAHGSIQFENLIYRGEFLKAHKGEYV
TLRYDPDHILSLYIYSGETDDNAGEFLGYAHAVNMDTHDLSIEELKALNKERSNA
RKEHFNYDALLALGKRKELVEERKEDKKAKRNSEQKRLRSASKKNSNVIELRKS
RTSKSLKKQENQEVLPERISREEIKLEKIEQQPQENLSASPNTQEEERHKLVFSNR
QKNLNKIW
nAniI-XTEN18-ShoTnsB: nicking I-AniI fused to ShoCAST TnsB with an 18 amino acid
XTEN linker
(SEQ ID NO: 97)
MGSDLTYAYLVGLFEGDGYFSITKKGKYLTYELGIELSIKDVQLIYKIKKIL
GIGIVSFRKRNEIEMVALRIRDKNHLKSKILPIFEKYPMFSNKQYDYLRFRNALLS
GIISLEDLPDYTRSDEPLNSIESIINTSYFSAWLVGFIEAEGCFSVYKLNKDDDYLIA
SFDIAQRDGDILISAIRKYLSFTTKVYLDKTNCSKLKVTSVRSVENIIKFLQNAPVK
LLGNMKLQYKLWLKQLRKISRYSEKIKIPSNYSGSETPGTSESATPESGSGLDEEF
EFTEELTQAPDVIVLDKSHFVVDPSQIILQTSDKHKLRFNLIKWFAESPNITIKSQR
KQAVVDTLGVSTRQVERLLKQYHNGELSETAGVQRSDKGKLRISQYWEDYIKTT
YEKSLKDKHPMLPAAVVREVKRHAIVDLGLKPGDYPHPATIYRNLAPLIEQHTR
KKKVRNPGSGSWLTVVTRDGQLLKADFSNQIIQCDHTELDIHIVDSHGSLLSDRP
WLTTVVDTYSSCILGFHLWIKQPGSTEVALALRHAILPKNYPEDYKLGKVWEIYG
PPFQYFFTDGGKDFNSKHLKAIGKKLGFQCELRNRPPQGGIVERLFKTINTQVLK
ELPGYTGANVQERPKNAEKEACLTIQDLDKILASFFCDIYNHEPYPKEPRNTRFER
WFKGMGGKLPEPLDERELDICLMKEAQRVVQAHGSIQFENLIYRGEALKAYRGE
YVTLRYDPDHVLTLYVYSCEADDNAEEFLGYAHAINMDTHDLSIEELKTLNKER
SKARSDHYNYDALLALGKRKELVEERKQDKKAKRQSEQKRLRTASKKNSNVIE
LRKSRASSSSSKDDRQEILPERVSRDELKPEKTELKYEENLLAQTDTQKQERHKL
VVSDRKKNLKNIW
nAniI-XTEN18-N7TnsB: nicking NLS-I-AniI fused to N7CAST TnsB with an 18 amino
acid XTEN linker
(SEQ ID NO: 98)
MYPYDVPDYAGGGSGPKKKRKVGGGSGGSDLTYAYLVGLFEGDGYFSIT
KKGKYLTYELGIELSIKDVQLIYKIKKILGIGIVSFRKRNEIEMVALRIRDKNHLKS
KILPIFEKYPMFSNKQYDYLRFRNALLSGIISLEDLPDYTRSDEPLNSIESIINTSYFS
AWLVGFIEAEGCFSVYKLNKDDDYLIASFDIAQRDGDILISAIRKYLSFTTKVYLD
KTNCSKLKVTSVRSVENIIKFLQNAPVKLLGNMKLQYKLWLKQLRKISRYSEKIK
IPSNYSGSETPGTSESATPESGSDEMPIVKQDDESLPVENNDDVDEIQDDELEETN
VIFTELSAEAKLKMDVIQGLLEPCDRKTYGEKLRVAAEKLGKTVRTVQRLVKKY
QQDGLSAIVETQRNDKGSYRIDPEWQKFIVNTFKEGNKGSKKMTPAQVAMRVQ
VRAEQLGLQKFPSHMTVYRVLNPIIERQERKQKQRNIGWRGSRVSHKTRDGQTL
DVRYSNHVWQCDHTKLDVMLVDQYGEPLARPWFTKITDSYSRCIMGIHVGFDA
PSSQVVALASRHAILPKQYSAEYKLISDWGTYGVPENLFTDGGRDFRSEHLKQIG
FQLGFECHLRDRPSEGGIEERSFGTINTEFLSGFYGYLGSNIQERSKTAEEEACLTL
RELHLLLVRYIVDNYNQRLDARTKDQTRFQRWEAGLPALPKMVKERELDICLM
KKTRRSIYKGGYLSFENIMYRGDYLAAYAGENIVLRYDPRDITTVWVYRIDKGK
EVFLSAAHALDWETEQLSLEEAKAASRKVRSVGKTLSNKSILAEIHDRDTFIKQK
KKSQKERKKEEQAQVHAVYEPINLSETEPLENLQETPKPVTRKPRIFNYEQLRQD
YDE
Cas12k fusions to make 3-component CASTs (TnsB not fused to anything) or 3-
component HELIX (nAniI-TnsB)
Cas12k-XTEN18-TniQ: ShCAST Cas12k fused to ShCAST TniQ via an 18 amino acid
XTEN linker; other two components are TnsB (or nAniI-TnsB for HELIX) and TnsC
(SEQ ID NO: 99)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRSSGSETPGTSESA
TPESGSIEAPDVKPWLFLIKPYEGESLSHFLGRFRRANHLSASGLGTLAGIGAIVA
RWERFHFNPRPSQQELEAIASVVEVDAQRLAQMLPPAGVGMQHEPIRLCGACYA
ESPCHRIEWQYKSVWKCDRHQLKILAKCPNCQAPFKMPALWEDGCCHRCRMPF
AEMAKLQKV
Cas12k-XTEN18-TniQ-3xGGGS (SEQ ID NO: 157)-TniQ: ShCAST Cas12k fused to
ShCAST TniQ via an 18 amino acid XTEN linker. The two TniQs are fused via a
3x(GGGS) linker (SEQ ID NO: 157); other two components are TnsB (or nAniI-TnsB for
HELIX) and TnsC
(SEQ ID NO: 100)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRSSGSETPGTSESA
TPESGSIEAPDVKPWLFLIKPYEGESLSHFLGRFRRANHLSASGLGTLAGIGAIVA
RWERFHFNPRPSQQELEAIASVVEVDAQRLAQMLPPAGVGMQHEPIRLCGACYA
ESPCHRIEWQYKSVWKCDRHQLKILAKCPNCQAPFKMPALWEDGCCHRCRMPF
AEMAKLQKVGGGSGGGSGGGSIEAPDVKPWLFLIKPYEGESLSHFLGRFRRANH
LSASGLGTLAGIGAIVARWERFHFNPRPSQQELEAIASVVEVDAQRLAQMLPPAG
VGMQHEPIRLCGACYAESPCHRIEWQYKSVWKCDRHQLKILAKCPNCQAPFKM
PALWEDGCCHRCRMPFAEMAKLQKV
Cas12k-XTEN18-TnsC: ShCAST Cas12k fused to ShCAST TnsC via an 18 amino acid XTEN
linker; other two comopnents are TnsB (or nAniI-InsB for HELIX) and TniQ
(SEQ ID NO: 101)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRSSGSETPGTSESA
TPESGSTEAQAIAKQLGGVKPDDEWLQAEIARLKGKSIVPLQQVKTLHDWLDGK
RKARKSCRVVGESRTGKTVACDAYRYRHKPQQEAGRPPTVPVVYIRPHQKCGP
KDLFKKITEYLKYRVTKGTVSDFRDRTIEVLKGCGVEMLIIDEADRLKPETFADV
RDIAEDLGIAVVLVGTDRLDAVIKRDEQVLERFRAHLRFGKLSGEDFKNTVEMW
EQMVLKLPVSSNLKSKEMLRILTSATEGYIGRLDEILREAAIRSLSRGLKKIDKAV
LQEVAKEYK
Cas12k-XTEN18-TniQ-3xGGGS (SEQ ID NO: 157)-InsC: ShCAST Cas12k fused to
ShCAST TniQ via an 18 amino acid XTEN linker fused to ShCAST TnsC via a 3x(GGGS)
(SEQ ID NO: 157) linker
(SEQ ID NO: 102)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRSSGSETPGTSESA
TPESGSIEAPDVKPWLFLIKPYEGESLSHFLGRFRRANHLSASGLGTLAGIGAIVA
RWERFHFNPRPSQQELEAIASVVEVDAQRLAQMLPPAGVGMQHEPIRLCGACYA
ESPCHRIEWQYKSVWKCDRHQLKILAKCPNCQAPFKMPALWEDGCCHRCRMPF
AEMAKLQKVGGGSGGGSGGGSTEAQAIAKQLGGVKPDDEWLQAEIARLKGKSI
VPLQQVKTLHDWLDGKRKARKSCRVVGESRTGKTVACDAYRYRHKPQQEAGR
PPTVPVVYIRPHQKCGPKDLFKKITEYLKYRVTKGTVSDFRDRTIEVLKGCGVEM
LIIDEADRLKPETFADVRDIAEDLGIAVVLVGTDRLDAVIKRDEQVLERFRAHLRF
GKLSGEDFKNTVEMWEQMVLKLPVSSNLKSKEMLRILTSATEGYIGRLDEILREA
AIRSLSRGLKKIDKAVLQEVAKEYK
Cas12k-XTEN18-TnsC-3xGGGS (SEQ ID NO: 157)-TniQ: ShCAST Cas12k fused to
ShCAST TnsC via an 18 amino acid XTEN linker fused to ShCAST TniQ via a 3x(GGGS)
(SEQ ID NO: 157) linker
(SEQ ID NO: 103)
MSQITIQARLISFESNRQQLWKLMADLNTPLINELLCQLGQHPDFEKWQQ
KGKLPSTVVSQLCQPLKTDPRFAGQPSRLYMSAIHIVDYIYKSWLAIQKRLQQQL
DGKTRWLEMLNSDAELVELSGDTLEAIRVKAAEILAIAMPASESDSASPKGKKG
KKEKKPSSSSPKRSLSKTLFDAYQETEDIKSRSAISYLLKNGCKLTDKEEDSEKFA
KRRRQVEIQIQRLTEKLISRMPKGRDLTNAKWLETLLTATTTVAEDNAQAKRWQ
DILLTRSSSLPFPLVFETNEDMVWSKNQKGRLCVHFNGLSDLIFEVYCGNRQLH
WFQRFLEDQQTKRKSKNQHSSGLFTLRNGHLVWLEGEGKGEPWNLHHLTLYCC
VDNRLWTEEGTEIVRQEKADEITKFITNMKKKSDLSDTQQALIQRKQSTLTRINN
SFERPSQPLYQGQSHILVGVSLGLEKPATVAVVDAIANKVLAYRSIKQLLGDNYE
LLNRQRRQQQYLSHERHKAQKNFSPNQFGASELGQHIDRLLAKAIVALARTYKA
GSIVLPKLGDMREVVQSEIQAIAEQKFPGYIEGQQKYAKQYRVNVHRWSYGRLI
QSIQSKAAQTGIVIEEGKQPIRGSPHDKAKELALSAYNLRLTRRSSGSETPGTSESA
TPESGSTEAQAIAKQLGGVKPDDEWLQAEIARLKGKSIVPLQQVKTLHDWLDGK
RKARKSCRVVGESRTGKTVACDAYRYRHKPQQEAGRPPTVPVVYIRPHQKCGP
KDLFKKITEYLKYRVTKGTVSDFRDRTIEVLKGCGVEMLIIDEADRLKPETFADV
RDIAEDLGIAVVLVGTDRLDAVIKRDEQVLERFRAHLRFGKLSGEDFKNTVEMW
EQMVLKLPVSSNLKSKEMLRILTSATEGYIGRLDEILREAAIRSLSRGLKKIDKAV
LQEVAKEYKGGGSGGGSGGGSIEAPDVKPWLFLIKPYEGESLSHFLGRFRRANHL
SASGLGTLAGIGAIVARWERFHFNPRPSQQELEAIASVVEVDAQRLAQMLPPAGV
GMQHEPIRLCGACYAESPCHRIEWQYKSVWKCDRHQLKILAKCPNCQAPFKMP
ALWEDGCCHRCRMPFAEMAKLQKV
pDONOR sequences without I-AniI sites (LE underlined and RE italicized)
ShCAST pDonor (no I-AniI site) with native flanking sequences
(SEQ ID NO: 104)
TTAGACATCTCCACAAAAGGCGTAGTGTACAGTGACAAATTATCTGTCGTCGGTGACAGATTAATGTCATT
GTGACTATTTAATTGTCGTCGTGACCCATCAGCGTTGCTTAATTAATTGATGACAAATTAAATGTCATCAA
TATAATATGCTCTGCAATTATTATACAAAGCAATTAAAACAAGCGGATAAAAGGACTTGCTTTCAACCCAC
CCCTAAGTTTAATAGTTACTGA[CARGO]GCGACAGTCAATTTGTCATTATGAAAATACACAAAAGCTTTT
TCCTATCTTGCAAAGCGACAGCTAATTTGTCACAATCACGGACAACGACATCTATTTTGTCACTGCAAAGA
GGTTATGCTAAAACTGCCAAAGCGCTATAATCTATACTGTATAAGGATTTTACTGATGACAATAATTTGTC
ACAACGACATATAATTAGTCACTGTACACGTAGAGACGTAGCAATGCTACCTC
AcCAST pDonor (no I-AniI site) with native flanking sequences
(SEQ ID NO: 105)
CGAGTCTCCTATTCTCCATTATATATGTACATTCGCAAATTAAATGTCGCTTTTCGCAATTTAGTGTCGTT
ATTCGCAAATTAATGTCGTGGTGGTTGTTTTTCAGAGTCAATTTAATTATTCTAAGTTTTCGCAAATTAAT
GTCGCATGAACTTAACATTTACTATACAATAAATTATTGCTGCAAGGGCATTATTGGATTATTGATATGTG
TTCGATCGCAGCACTCCT[CARGO]GACATCTAATTTGCAAAATACCAAATTCTTAACAAACGACATTTAA
TTTGCGAAACCAGGTTTTACGACATACAATATGCGAATTAGGTAACTTAGTCTTTTGTAGGGGTAAATAGC
TTATGATGCTTATAGAATAAAGGTTTTAGTCCTTAAAAGCAGTTGCGACACTAATTTGCGAAAAGCGACAT
TTAATTTGCGAACGTACAATAGCCTTTCTCACTCTAGTTAGAT
ShoCAST pDonor (no I-AniI site) with ShCAST flanking sequences
(SEQ ID NO: 106)
TTAGACATCTCCACAAAAGGCGTAGTGTACATTCGCAAATTAAATGTCGTAATTCGCAAATTTGTGTCGTT
TTTCGCAAATTAATGTCGTTTAGAATAGTTTGTCTCATCAATTCAATTATAGGAACTTTTCGCAAATTAAT
GTCGTCCTGTTTCTCCATTTAGTGTCGATTAACAAATTAATGTCGCTGTTAACGAATTAATGTCGTCGAAT
TAGTTCCAACTAACG[CARGO]GACATCTAATTTGCGAAACAGGCAAATCTTAATAAACGACATTTAATTT
GCGAAAATAGGATTTGCGACATCTAATTTGCGAAACAGGCAAATTACTCAGTTTTATGGATAAATAGCTTG
TAAGTCCTACGCAATAAAGATCTCAGCTATTAGAAGTAATTGCGACACTAATTTGCGAATTGCGACATATA
ATTTGCGAATGTACACGTAGAGACGTAGCAATGCTACCTC
AcCAST pDonor (no I-AniI site) with ShCAST flanking sequences
(SEQ ID NO: 107)
TTAGACATCTCCACAAAAGGCGTAGTGTACATTCGCAAATTAAATGTCGCTTTTCGCAATTTAGTGTCGTT
ATTCGCAAATTAATGTCGTGGTGGTTGTTTTTCAGAGTCAATTTAATTATTCTAAGTTTTCGCAAATTAAT
GTCGCATGAACTTAACATTTACTATACAATAAATTATTGCTGCAAGGGCATTATTGGATTATTGATATGTG
TTCGATCGCAGCACTCCT[CARGO]GACATCTAATTTGCAAAATACCAAATTCTTAACAAACGACATTTAA
TTTGCGAAACCAGGTTTTACGACATACAATATGCGAATTAGGTAACTTAGTCTTTTGTAGGGGTAAATAGC
TTATGATGCTTATAGAATAAAGGTTTTAGTCCTTAAAAGCAGTTGCGACACTAATTTGCGAAAAGCGACAT
TTAATTTGCGAACGTACACGTAGAGACGTAGCTAATGCTACCTC
N7CAST pDonor (no I-AniI site) with native flanking sequences and 400 bp
of LE/RE (not minimized)
(SEQ ID NO: 108)
AAATCCAGCTGCTGGCTTTAACTTATGTCGAATAACTAATTATTTGTCGTTGTTAACAGATTGCTGTCGCT
ATTAACAAATTAATGTCACTGTTAACAAATTAGTGTCGTATAATGCTAATTGCGAAACGTTAACAAATTAA
TGTCGTCTAACCAATTTGATAAAGTGTTTGCAGACATCTATTGTACAGGAAATATAGCTAAATCTTTATTT
GATGACTTCCCTGATAATATTCATAAATATGCTTACAAGTCGGATGCACCTTTCAACCCTCTGTTAAATAT
TTTCTGACGCTCTTTCAACTCATCCCTAGCTGGGATAGTTGTTGAAACTTAGAGTCACCCAGTTTGGCATT
AGATACTATCTTTTTTCAACCTACCCCTAACCAGGATGGTCGTTGAAACCTGGATATGCTCAATACAAGG-
[CARGO]AAAACTTGATTCATACTCAAAACAGTAATCACAATCTCGCTATTGTGCGAGAACATCCAAACTT
CCTAAAGCAGTTGACCCCTCAATGGACGCGGCAACTTTTCGGTATAAGGATGTATTATTTAGTGCAAATGT
ACTAAATAAAATTATAATACCACTATTCAAGCTAAAAAGCGACAGCTAATTTGTTATGAAACTAGAAAATT
TTAGAAAACGTAAAATTTTAAAAGACGACGTTTATTTTGTTATTATTTAAATCAACGACAAGTAAAGTGTT
AAATAAACTACTAACCCATTACATAATAAAAAACGTTGTAAACACTCATGTAGCAACATTTTTGATAGTTT
TATATTTGACGACATTATTTTGTTAAGACGACAAATAATTAGTTATTCAACAACTTAAATTTATCTGCATT
TAATTG

TABLE 4
Additional Sequences

| fusion | amino acid or | | |
|---|---|---|---|
| protein | ribonucleotide | description | sequence |
| E. coli | amino acid | Ribosomal | MSLSTEATAKIVSEFGRDANDTGS |
| S15 | | Protein S15 | TEVQVALLTAQINHLQGHFAEHK |
| | | from E. coli | KDHHSRRGLLRMVSQRRKLLDY |
| | | | LKRKDVARYTQLIERLGLRR |
| | | | (SEQ ID NO: 109) |
| N7 S15 | amino acid | Ribosomal | MALTQQRKQEIITNFQVHETDTGS |
| | | Protein S15 | ADVQIAMLTERINRLSEHLQANK |
| | | from Nostoc | KDHSSRRGLLKLIGHRKRLLAYL |
| | | Sp. PCC7107 | QQESREKYQALIARLGIRG (SEQ |
| | | | ID NO: 110) |
| Ac S15 | amino acid | Ribosomal | MALTQQRKQELISGYQVHETDTG |
| | | Protein S15 | SADVQIAMLTDRINRLSQHLQAN |
| | | from A. | KKDHSSRRGLLKMIGQRKRLLSYI |
| | | cylindrica | QKGSREKYQALIARLGIRG (SEQ |
| | | | ID NO: 111) |
| Sh S15 | amino acid | Ribosomal | MALTQERKQEIIVNYQVHETDTG |
| | | Protein S15 | SADVQVAMLTERINRLSLHLQAN |
| | | from S. | KKDHSSRRGLLKLIGQRKRLLAYI |
| | | Hofmanni | QKDSREKYQALIGRLGIRG (SEQ |
| | | | ID NO: 112) |
| pi protein | amino acid | pi protein | MRLKVMMDVNKKTKIRHRNELN |
| | | from the pir | HTLAQLPLPAKRVMYMALAPIDS |
| | | gene (in PIR2 | KEPLERGRVFKIRAEDLAALAKIT |
| | | cells) | PSLAYRQLKEGGKLLGASKISLRG |
| | | | DDIIALAKELNLPFTAKNSPEELD |
| | | | LNIIEWIAYSNDEGYLSLKFTRTIE |
| | | | PYISSLIGKKNKFTTQLLTASLRLS |
| | | | SQYSSSLYQLIRKHYSNFKKKNYF |
| | | | IISVDELKEELIAYTFDKDGNIEYK |
| | | | YPDFPIFKRDVLNKAIAEIKKKTEI |
| | | | SFVGFTVHEKEGRKISKLKFEFVV |
| | | | DEDEFSGDKDDEAFFMNLSEADA |
| | | | AFLKVFDETVPPKKAKG (SEQ ID |
| | | | NO: 113) |
| E. coli HU | amino acid | HU Protein | MNKTQLIDVIAEKAELSKTQAKA |
| Alpha | | chain Alpha | ALESTLAAITESLKEGDAVQLVGF |
| | | from E. coli | GTFKVNHRAERTGRNPQTGKEIKI |
| | | | AAANVPAFVSGKALKDAVK |
| | | | (SEQ ID NO: 114) |
| E. coli HU | amino acid | HU Protein | MNKSQLIDKIAAGADISKAAAGR |
| Beta | | chain Beta | ALDAIIASVTESLKEGDDVALVGF |
| | | from E. coli | GTFAVKERAARTGRNPQTGKEITI |
| | | | AAAKVPSFRAGKALKDAVN (SEQ |
| | | | ID NO: 115) |
| E. coli HU | amino acid | HU Protein | NKTQLIDVIAEKAELSKTQAKAA |
| Single | | from E. coli | LESTLAAITESLKEGDAVQLVGFG |
| Chain | | single chain, | TFKVNHRAERTGRNPQTGKEIKIA |
| (Alpha- | | Alpha-Beta | AANVPAFVSGKALKDAVKSGSGS |
| Beta) | | fused with | ETPGTSESATPESGSGSNKSQLIDK |
| | | XTEN linker | IAAGADISKAAAGRALDAIIASVT |
| | | | ESLKEGDDVALVGFGTFAVKERA |
| | | | ARTGRNPQTGKEITIAAAKVPSFR |
| | | | AGKALKDAVN (SEQ ID NO: 116) |
| N7 HU | amino acid | HU from | MNKGELVDAVAEKASVTKKQAD |
| | | Nostoc Sp. | AVLTAALETIIEAVSSGDKVTLVG |
| | | PCC7107 | FGSFESRERKAREGRNPKTNEKM |
| | | | EIPATKVPAFSAGKLFRERVAPPK |
| | | | S (SEQ ID NO: 117) |
| Ac HU | amino acid | HU from A. | MNKGELVDAVAEKASVTKKQAD |
| | | cylindrica | AVLSAALETIIEAVSSGDKVTLVG |
| | | | FGSFESRERKAREGRNPKTNEKM |
| | | | EIPATKVPAFSAGKMFRERVAPPK |
| | | | E (SEQ ID NO: 118) |
| Sh HU | amino acid | HU from S. | MNKGELVDAVAEKASVTKKQAD |
| | | Hofmanni | AVLSAALETIIEAVSSGDKVTLVG |
| | | | FGSFESRERKAREGRNPKTNEKM |
| | | | EIPATKVPAFSAGKMFRERVAPPK |
| | | | V (SEQ ID NO: 119) |
| E. coli | amino acid | IHF Protein | MALTKAEMSEYLFDKLGLSKRD |
| IHF A | | chain A from | AKELVELFFEEIRRALENGEQVKL |
| | | E. coli | SGFGNFDLRDKNQRPGRNPKTGE |
| | | | DIPITARR VVTFRPGQKLKSRVEN |
| | | | ASPKDE (SEQ ID NO: 120) |
| E. coli | amino acid | IHF Protein | MTKSELIERLATQQSHIPAKTVED |
| IHF B | | chain B from | AVKEMLEHMASTLAQGERIEIRG |
| | | E. coli | FGSFSLHYRAPRTGRNPKTGDKV |
| | | | ELEGKYVPHFKPGKELRDRANIY |
| | | | G (SEQ ID NO: 121) |
| E. coli | amino acid | IHF Protein | MTKSELIERLATQQSHIPAKTVED |
| IHF single | | from E. coli | AVKEMLEHMASTLAQGERIEIRG |
| chain (B- | | single chain, | FGSFSLHYRAPRTGRNPKTGDKV |
| A) | | B-A fused | ELEGKYVPHFKPGKELRDRANIY |
| | | with XTEN | GSGSGSETPGTSESATPESGSGSA |
| | | linker | LTKAEMSEYLFDKLGLSKRDAKE |
| | | | LVELFFEEIRRALENGEQVKLSGF |
| | | | GNFDLRDKNQRPGRNPKTGEDIPI |
| | | | TARRVVTFRPGQKLKSRVENASP |
| | | | KDE (SEQ ID NO: 122) |
| E. coli | amino acid | IHF Protein | MALTKAEMSEYLFDKLGLSKRD |
| IHF single | | from E. coli | AKELVELFFEEIRRALENGEQVKL |
| chain (A- | | single chain, | SGFGNFDLRDKNQRPGRNPKTGE |
| B) | | A-B fused | DIPITARRVVTFRPGQKLKSRVEN |
| | | with XTEN | ASPKDESGSGSETPGTSESATPES |
| | | linker | GSGSTKSELIERLATQQSHIPAKT |
| | | | VEDAVKEMLEHMASTLAQGERIE |
| | | | IRGFGSFSLHYRAPRTGRNPKTGD |
| | | | KVELEGKYVPHFKPGKELRDRAN |
| | | | IYG (SEQ ID NO: 123) |

### REFERENCES FOR EXAMPLES 1-6

1. Hendrie, P. C. & Russell, D. W. Gene Targeting with Viral Vectors. Mol. Ther. 12, 9-17 (2005).
2. Thomas, C. E., Ehrhardt, A. & Kay, M. A. Progress and problems with the use of viral vectors for gene therapy. Nat. Rev. Genet. 4, 346-358 (2003).
3. Tellier, M., Bouuaert, C. C. & Chalmers, R. Mariner and the ITm Superfamily of Transposons. Microbiol. Spectr. 3, 3.2.06 (2015).
4. van Opijnen, T. & Camilli, A. Transposon insertion sequencing: a new tool for systems-level analysis of microorganisms. Nat. Rev. Microbiol. 11, 435-442 (2013).
5. Haniford, D. B. & Ellis, M. J. Transposons Tn 10 and Tn 5. Microbiol. Spectr. 3, 3.1.06 (2015).
6. Plasterk, R. H. A., Izsvák, Z. & Ivics, Z. Resident aliens: the Tc1/mariner superfamily of transposable elements. Trends Genet. 15, 326-332 (1999).
7. Wilson, M. H., Coates, C. J. & George, A. L. PiggyBac Transposon-mediated Gene Transfer in Human Cells. Mol. Ther. 15, 139-145 (2007).
8. Mali, P. et al. RNA-Guided Human Genome Engineering via Cas9. Science 339, 823-826 (2013).
9. Cong, L. et al. Multiplex Genome Engineering Using CRISPR/Cas Systems. Science 339, 819-823 (2013).
10. Rouet, P., Smih, F. & Jasin, M. Expression of a site-specific endonuclease stimulates homologous recombination in mammalian cells. Proc. Natl. Acad. Sci. 91, 6064-6068 (1994).
11. Wang, H. H. et al. Programming cells by multiplex genome engineering and accelerated evolution. Nature 460, 894-898 (2009).
12. Wang, H. H. et al. Genome-scale promoter engineering by coselection MAGE. Nat. Methods 9, 591-593 (2012).
13. Jiang, W., Bikard, D., Cox, D., Zhang, F. & Marraffini, L. A. RNA-guided editing of bacterial genomes using CRISPR-Cas systems. Nat. Biotechnol. 31, 233-239 (2013).
14. Strecker, J. et al. RNA-guided DNA insertion with CRISPR-associated transposases. Science 365, 48-53 (2019).
15. Klompe, S. E., Vo, P. L. H., Halpin-Healy, T. S. & Sternberg, S. H. Transposon-encoded CRISPR-Cas systems direct RNA-guided DNA integration. Nature 571, 219-225 (2019).
16. Vo, P. L. H. et al. CRISPR RNA-guided integrases for high-efficiency, multiplexed bacterial genome engineering. Nat. Biotechnol. 39, 480-489 (2021).
17. Vo, P. L. H., Acree, C., Smith, M. L. & Sternberg, S. H. Unbiased profiling of CRISPR RNA-guided transposition products by long-read sequencing. Mob. DNA 12, 13 (2021).
18. Saito, M. et al. Dual modes of CRISPR-associated transposon homing. Cell 184, 2441-2453.e18 (2021).
19. Strecker, J., Ladha, A., Makarova, K. S., Koonin, E. V. & Zhang, F. Response to Comment on “RNA-guided DNA insertion with CRISPR-associated transposases”. Science 368, eabb2920 (2020).
20. Rubin, B. E. et al. Species- and site-specific genome editing in complex bacterial communities. Nat. Microbiol. 7, 34-47 (2022).
21. Rybarski, J. R., Hu, K., Hill, A. M., Wilke, C. O. & Finkelstein, I. J. Metagenomic discovery of CRISPR-associated transposons. Proc. Natl. Acad. Sci. 118, e2112279118 (2021).
22. May, E. W. & Craig, N. L. Switching from Cut-and-Paste to Replicative Tn7 Transposition. Science 272, 401-404 (1996).
23. Kholodii, G. Ya. et al. Four genes, two ends, and a res region are involved in transposition of Tn5053: a paradigm for a novel family of transposons carrying either a mer operon or an integron. Mol. Microbiol. 17, 1189-1200 (1995).
24. Hickman, A. B. et al. Unexpected Structural Diversity in DNA Recombination. Mol. Cell 5, 1025-1034 (2000).
25. Xu, S. Sequence-specific DNA nicking endonucleases. Biomol. Concepts 6, 253-267 (2015).
26. Gasiunas, G., Barrangou, R., Horvath, P. & Siksnys, V. Cas9-crRNA ribonucleoprotein complex mediates specific DNA cleavage for adaptive immunity in bacteria. Proc. Natl. Acad. Sci. 109, (2012).
27. Jinek, M. et al. A Programmable Dual-RNA-Guided DNA Endonuclease in Adaptive Bacterial Immunity. Science 337, 816-821 (2012).
28. Xu, S. & Gupta, Y. K. Natural zinc ribbon HNH endonucleases and engineered zinc finger nicking endonuclease. Nucleic Acids Res. 41, 378-390 (2013).
29. McConnell Smith, A. et al. Generation of a nicking enzyme that stimulates site-specific gene conversion from the I-AniI LAGLIDADG homing endonuclease. Proc. Natl. Acad. Sci. 106, 5099-5104 (2009).
30. Niu, Y., Tenney, K., Li, H. & Gimble, F. S. Engineering variants of the I-SceI homing endonuclease with strand-specific and site-specific DNA-nicking activity. J. Mol. Biol. 382, 188-202 (2008).
31. Kong, S., Liu, X., Fu, L., Yu, X. & An, C. I-PfoP3I: a novel nicking HNH homing endonuclease encoded in the group I intron of the DNA polymerase gene in Phormidium foveolarum phage Pf-WMP3. PloS One 7, e43738 (2012).
32. Landthaler, M. & Shub, D. A. The nicking homing endonuclease I-BasI is encoded by a group I intron in the DNA polymerase gene of the Bacillus thuringiensis phage Bastille. Nucleic Acids Res. 31, 3071-3077 (2003).
33. Shen, Y. et al. Structural basis for DNA targeting by the Tn7 transposon. Nat. Struct. Mol. Biol. 29, 143-151 (2022).
34. Stoddard, B. L. Homing endonucleases from mobile group I introns: discovery to genome engineering. Mob. DNA 5, 7 (2014).
35. Takeuchi, R., Certo, M., Caprara, M. G., Scharenberg, A. M. & Stoddard, B. L. Optimization of in vivo activity of a bifunctional homing endonuclease and maturase reverses evolutionary degradation. Nucleic Acids Res. 37, 877-890 (2009).
36. Querques, I., Schmitz, M., Oberli, S., Chanez, C. & Jinek, M. Target site selection and remodelling by type V CRISPR-transposon systems. Nature 599, 497-502 (2021).
37. Park, J.-U., Tsai, A. W.-L., Chen, T. H., Peters, J. E. & Kellogg, E. H. Mechanistic details of CRISPR-associated transposon recruitment and integration revealed by cryo-EM. Proc. Natl. Acad. Sci. U.S.A 119, e2202590119 (2022).
38. Tenjo-Castaño, F. et al. Structure of the TnsB transposase-DNA complex of type V-K CRISPR-associated transposon. http://biorxiv.org/lookup/doi/10.1101/2022.08.05.502904 (2022) doi:10.1101/2022.08.05.502904.
39. Liu, R., Qiu, J., Finger, L. D., Zheng, L. & Shen, B. The DNA-protein interaction modes of FEN-1 with gap substrates and their implication in preventing duplication mutations. Nucleic Acids Res. 34, 1772-1784 (2006).
40. Scalley-Kim, M., McConnell-Smith, A. & Stoddard, B. L. Coevolution of a Homing Endonuclease and Its Host Target Sequence. J. Mol. Biol. 372, 1305-1319 (2007).
41. Gilpatrick, T. et al. Targeted nanopore sequencing with Cas9-guided adapter ligation. Nat. Biotechnol. 38, 433-438 (2020).
42. Park, J.-U. et al. Structural basis for target site selection in RNA-guided DNA transposition systems. Science 373, 768-774 (2021).
43. Schmitz, M., Querques, I., Oberli, S., Chanez, C. & Jinek, M. Structural basis for RNA-mediated assembly of type V CRISPR-associated transposons. http://biorxiv.org/lookup/doi/10.1101/2022.06.17.496590 (2022) doi:10.1101/2022.06.17.496590.
44. Mizuno, N. et al. MuB is an AAA+ ATPase that forms helical filaments to control target selection for DNA transposition. Proc. Natl. Acad. Sci. 110, (2013).
45. Skelding, Z., Queen-Baker, J. & Craig, N. L. Alternative interactions between the Tn7 transposase and the Tn7 target DNA binding protein regulate target immunity and transposition. EMBO J. 22, 5904-5917 (2003).
46. Stellwagen, A. E. & Craig, N. L. Avoiding self: two Tn7-encoded proteins mediate target immunity in Tn7 transposition. EMBO J. 16, 6823-6834 (1997).
47. Kolter, R., Inuzuka, M. & Helinski, D. R. Trans-complementation-dependent replication of a low molecular weight origin fragment from plasmid R6K. Cell 15, 1199-1208 (1978).
48. Metcalf, W. W., Jiang, W. & Wanner, B. L. Use of the rep technique for allele replacement to construct new Escherichia coli hosts for maintenance of R6K gamma origin plasmids at different copy numbers. Gene 138, 1-7 (1994).
49. Klompe, S. E. et al. Evolutionary and mechanistic diversity of Type I-F CRISPR-associated transposons. Mol. Cell 82, 616-628.e5 (2022).
50. Jonathan Strecker, Feng Zhang, Alim Ladha. Crispr-associated transposase systems and methods of use thereof.
51. Harshey, R. M. Transposable Phage Mu. Microbiol. Spectr. 2, (2014).
52. Wu, Z. & Chaconas, G. Flanking host sequences can exert an inhibitory effect on the cleavage step of the in vitro mu DNA strand transfer reaction. J. Biol. Chem. 267, 9552-9558 (1992).
53. Krüger, R. & Filutowicz, M. Dimers of pi protein bind the A+T-rich region of the R6K gamma origin near the leading-strand synthesis start sites: regulatory implications. J. Bacteriol. 182, 2461-2467 (2000).
54. Chalmers, R., Guhathakurta, A., Benjamin, H. & Kleckner, N. IHF modulation of Tn10 transposition: sensory transduction of supercoiling status via a proposed protein/DNA molecular spring. Cell 93, 897-908 (1998).
55. Swingle, B., O'Carroll, M., Haniford, D. & Derbyshire, K. M. The effect of host-encoded nucleoid proteins on transposition: H—NS influences targeting of both IS903 and Tn10. Mol. Microbiol. 52, 1055-1067 (2004).
56. Zayed, H., Izsvák, Z., Khare, D., Heinemann, U. & Ivics, Z. The DNA-bending protein HMGB1 is a cellular cofactor of Sleeping Beauty transposition. Nucleic Acids Res. 31, 2313-2322 (2003).
57. Filutowicz, M. & Appelt, K. The integration host factor of Escherichia coli binds to multiple sites at plasmid R6K gamma origin and is essential for replication. Nucleic Acids Res. 16, 3829-3843 (1988).
58. Sharpe, P. L. & Craig, N. L. Host proteins can stimulate Tn7 transposition: a novel role for the ribosomal protein L29 and the acyl carrier protein. EMBO J. 17, 5822-5831 (1998).
59. Parks, A. R. et al. Transposition into replicating DNA occurs through interaction with the processivity factor. Cell 138, 685-695 (2009).
60. Strecker, J. et al. Engineering of CRISPR-Cas12b for human genome editing. Nat. Commun. 10, 212 (2019).
61. Xu, X. et al. Engineered miniature CRISPR-Cas system for mammalian genome regulation and editing. Mol. Cell 81, 4333-4345.e4 (2021).
62. Kim, D. Y. et al. Efficient CRISPR editing with a hypercompact Cas12f1 and engineered guide RNAs delivered by adeno-associated virus. Nat. Biotechnol. 40, 94-102 (2022).
63. Anzalone, A. V. et al. Programmable deletion, replacement, integration and inversion of large DNA sequences with twin prime editing. Nat. Biotechnol. 40, 731-740 (2022).
64. Ioannidi, E. I. et al. Drag-and-drop genome insertion without DNA cleavage with CRISPR-directed integrases. http://biorxiv.org/lookup/doi/10.1101/2021.11.01.466786 (2021) doi:10.1101/2021.11.01.466786.
65. BBMap—Bushnell B.—sourceforge.net/projects/bbmap/.
66. Li, H. Minimap2: pairwise alignment for nucleotide sequences. Bioinformatics 34, 3094-3100 (2018).
67. Rohland, N. & Reich, D. Cost-effective, high-throughput DNA sequencing libraries for multiplexed target capture. Genome Res. 22, 939-946 (2012).
68. Kleinstiver, B. P. et al. Engineered CRISPR-Cas12a variants with increased activities and improved targeting ranges for gene, epigenetic and base editing. Nat. Biotechnol. 37, 276-282 (2019).

### REFERENCES FOR EXAMPLES 7-11

1. Takeuchi, R., Certo, M., Caprara, M. G., Scharenberg, A. M. & Stoddard, B. L. Optimization of in vivo activity of a bifunctional homing endonuclease and maturase reverses evolutionary degradation. Nucleic Acids Res. 37, 877-890 (2009).
2. Scalley-Kim, M., McConnell-Smith, A. & Stoddard, B. L. Coevolution of a Homing Endonuclease and Its Host Target Sequence. J. Mol. Biol. 372, 1305-1319 (2007).
3. McConnell Smith, A. et al. Generation of a nicking enzyme that stimulates site-specific gene conversion from the I-AniI LAGLIDADG homing endonuclease. Proc. Natl. Acad. Sci. 106, 5099-5104 (2009).
4. Park, J.-U. et al. Structural basis for target site selection in RNA-guided DNA transposition systems. Science 373, 768-774 (2021).
5. Schmitz, M., Querques, I., Oberli, S., Chanez, C. & Jinek, M. Structural basis for RNA-mediated assembly of type V CRISPR-associated transposons. http://biorxiv.org/lookup/doi/10.1101/2022.06.17.496590 (2022) doi:10.1101/2022.06.17.496590.
6. Park, J.-U., Tsai, A. W.-L., Chen, T. H., Peters, J. E. & Kellogg, E. H. Mechanistic details of CRISPR-associated transposon recruitment and integration revealed by cryo-EM. Proc. Natl. Acad. Sci. U.S.A 119, e2202590119 (2022).
7. Jonathan Strecker, Feng Zhang, Alim Ladha. Crispr-associated transposase systems and methods of use thereof. US2020/0190487A1
8. Gao, Z., Herrera-Carrillo, E. & Berkhout, B. Delineation of the Exact Transcription Termination Signal for Type 3 Polymerase III. Mol. Ther.—Nucleic Acids 10, 36-44 (2018).
9. Rybarski, J. R., Hu, K., Hill, A. M., Wilke, C. O. & Finkelstein, I. J. Metagenomic discovery of CRISPR-associated transposons. Proc. Natl. Acad. Sci. 118, e2112279118 (2021).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A fusion protein comprising a transposition protein B (TnsB) protein, e.g., Tn7, Tn7-like, or Tn5053-like transposition protein B (TnsB), fused (optionally via an intervening linker) to a protein (e.g., an endonuclease, e.g., a nickase, cleavase, or catalytically dead endonuclease, a fluorescent protein, or a peptide tag (e.g. NLS, His, Flag)).

2. The fusion protein of claim 1, wherein the endonuclease is a nickase, e.g., a homing endonuclease (HE), nicking restriction endonuclease, a nicking Cas variant, or a phage HNH endonuclease, or TnsA from a type I CAST or a Tn7 transposon, or a catalytic portion thereof.

3. The fusion protein of claim 2, wherein the HE is a LAGLIDADG, H—N—H, His-Cys box, or GIY-YIG HE.

4. The fusion protein of claim 3, wherein the HE is I-AniI, e.g., I-AniI from Aspergillus nidulans (I-AniI) or a variant thereof, optionally comprising a K227M mutation (nAniI), a hyperactive variant (e.g., Y2 I-AniI (F13Y, S111Y)), or both (K227M, F13Y, S111Y).

5. A nucleic acid comprising a sequence encoding the fusion protein of claim 1.

6. An expression construct comprising the nucleic acid of claim 5, and regulatory sequences to express the protein, e.g., a promoter.

7. An expression construct comprising sequences encoding a CRISPR-associated transposase (CAST), wherein the sequences comprise nucleic acids encoding the fusion protein of claim 1, Cas12k; TnsC; TniQ; optionally one or more host proteins; and a guide RNA (gRNA) that interacts with Cas12k and directs the Cas12k/gRNA complex to a target sequence, and regulatory sequences to express the sequences, e.g., one or more promoter sequences.

8. The expression construct of claim 7, wherein the Cas12k is fused to at least one other protein, optionally TniQ and/or TnsC (e.g., Cas12k-TniQ, Cas12k-TniQ-TniQ, Cas12k-TnsC, Cas12k-TniQ-TnsC, or Cas12k-TnsC-TniQ), optionally with a linker in between each protein.

9. The expression construct of claim 8, which is a plasmid or viral vector.

10. A host cell comprising and optionally expressing the nucleic acid of claim 5 comprising nucleic acid sequences encoding a Tn-endonuclease fusion protein, e.g., a TnsB-endonuclease fusion protein; and optionally one or more, e.g., all, of Cas12k; TnsC; TniQ; optionally one or more host proteins; and a guide RNA that binds to cas12k and directs the TnsB-endonuclease fusion protein to a selected target sequence, or a host cell comprising a CRISPR-associated transposase (CAST) comprising the fusion protein of claim 1; Cas12k; TnsC; TniQ; optionally one or more host proteins; and a gRNA that interacts with Cas12k and directs the fusion protein to a selected target sequence.

11. The host cell of claim 10, wherein the Cas12k is fused to at least one other protein, optionally TniQ (e.g., Cas12k-TniQ, TniQ-Cas12k, TniQ-TniQ-Cas12k, TniQ-Cas12k-TniQ, or Cas12k-TniQ-TniQ) and/or at least one TnsC, optionally with a linker in between each protein.

12. A method of inserting a desired sequence into DNA, e.g., into genomic DNA of a living cell, the method comprising expressing in the cell the nucleic acid of claim 5; Cas12k; TnsC; TniQ; optionally one or more host proteins; and a guide RNA that binds to cas12k and directs the endonuclease a selected target sequence, and a donor DNA molecule (e.g. a plasmid) comprising the desired sequence to be inserted, wherein the desired sequence is flanked by LE and RE flanking sequences on the 5′ and 3′ ends, respectively, and a target site for the endonuclease (e.g., I-AniI), preferably wherein the target site is oriented to confer a nick on the donor plasmid 5′ of the desired sequence to be inserted.

13. The method of claim 12, wherein the donor DNA molecule has modified LE/RE flanking sequences, e.g., a flanking sequence as shown in Table A that is from a source organism other than the source organism of at least one of the CAST components, i.e., TnsB; cas12k; TnsC; or TniQ, and/or comprising modifications or insertions at varying distances from the LE and RE sequences (e.g. an endonuclease recognition sequence or host factor binding sequence(s)).

14. The method of claim 13, wherein the modified LE/RE flanking sequences are from Scytonema hofmannii (e.g., from ShCAST), and wherein at least one of the Tn protein; cas12k; TnsC; or TniQ is from a CAST or HELIX ortholog (e.g. AcCAST and AcHELIX); are modified ShCAST LE/RE flanking sequences; or are de-novo LE/RE flanking sequences.

15. The method of claim 12, wherein the Cas12k is expressed as a fusion protein, optionally with at least one TniQ and/or at least one TnsC (e.g., Cas12k-TniQ, Cas12k-TniQ-TniQ, Cas12k-TnsC, Cas12k-TniQ-TnsC, or Cas12k-TnsC-TniQ), optionally with a linker in between each protein.

16. A fusion protein comprising:
Cas12k; optionally one or morehost proteins; and at least one TniQ (e.g., Cas12k-TniQ or
Cas12k-TniQ-TniQ) and/or at least one TnsC, optionally with a linker in between each segment.

17. A fusion protein comprising a host protein and one or more of Cas12k, TnsC, or TniQ, optionally with a linker in between each segment.

18. A composition comprising, or nucleic acids encoding:
(i) a fusion protein comprising a transposon (Tn) protein, e.g., Tn7, Tn7-like, or Tn5053-like, e.g., transposition protein B (TnsB), fused to a protein (e.g., an endonuclease, e.g., a nickase, cleavase, or catalytically dead endonuclease, a fluorescent protein, or a peptide tag (e.g. NLS, His, Flag)), optionally via an intervening linker; and
(ii) a fusion protein comprising a host protein and one or more of Cas12k, TnsC, or TniQ, optionally with a linker in between each segment.

19. A composition comprising, or nucleic acids encoding:
(ii) a fusion protein comprising a transposon (Tn) protein, e.g., Tn7, Tn7-like, or Tn5053-like, e.g., transposition protein B (TnsB), fused to a protein (e.g., an endonuclease, e.g., a nickase, cleavase, or catalytically dead endonuclease, a fluorescent protein, or a peptide tag (e.g. NLS, His, Flag)), optionally via an intervening linker; and
(ii) a fusion protein comprising a host protein and one or more of Cas12k, TnsC, or TniQ, optionally with a linker in between each segment.

20. The expression construct of any one of claim 7, the host cell of any one of claim 9, the methods of any one of claim 12, the fusion proteins of claim 16, or the composition of any one of claim 18, wherein the host factor is ribosomal protein S15, alters DNA topology (e.g., pi protein or a nucleoid-associated protein (NAP), such as, HU, Fis, H—NS, IHF, or TF1) or wherein the host factor is involved in DNA or cellular metabolism, proteolysis or protein folding, regulation, or transport (e.g., acyl carrier protein (ACP), Sigma S, DnaN, DnaA, DNA topoisomerase I, La protease, Dam methylase, or proteins expressed from the genes dcd, dinD, radA, recQ, clpX, fkpA, hflX, crl, rseB, rsxE, araJ, melB, mgtA, aspA, treC, proY, serA, yhbC, yidA, ykfA).

21. A host cell comprising or expressing the composition of any one of claim 18, and a donor DNA molecule (e.g. a plasmid) comprising the desired sequence to be inserted, wherein the desired sequence is flanked by LE and RE flanking sequences on the 5′ and 3′ ends, respectively, and a target site for the endonuclease (e.g., I-AniI), preferably wherein the target site is oriented to confer a nick on the donor plasmid 5′ of the desired sequence to be inserted.
